(19) United States
(12) Patent Application Publication
(10) Pub. No.: US 2025/0333755 A1
(43) Pub. Date: Oct. 30, 2025

(54) WHEAT WITH INCREASED RESISTANT STARCH LEVELS

(71) Applicant: Arcadia Biosciences, Inc., Dallas, TX (US)

(72) Inventors: Ann J. Slade, Bellevue, WA (US); Dayna L. Loeffler, Seattle, WA (US); Aaron M. Holm, Shoreline, WA (US); Jessica C. Mullenberg, Lynnwood, WA (US)

(21) Appl. No.: 19/204,085

(22) Filed: May 9, 2025

Related U.S. Application Data
(63) Continuation of application No. 18/298,041, filed on Apr. 10, 2023, now Pat. No. 12,319,920.
Application No. 18/298,041 is a continuation of application No. 17/147,281, filed on Jan. 12, 2021, now Pat. No. 11,649,464.
Application No. 17/147,281 is a continuation of application No. 15/914,994, filed on Mar. 7, 2018, now Pat. No. 10,934,557.
Application No. 15/914,994 is a continuation of application No. 14/825,369, filed on Aug. 13, 2015, now Pat. No. 10,563,217.
Application No. 14/825,369 is a continuation of application No. 13/633,588, filed on Oct. 2, 2012, now Pat. No. 9,150,839.
(60) Provisional application No. 61/542,953, filed on Oct. 4, 2011.

(51) Int. Cl. C12N 15/82; A01H 6/46; C12N 9/10

(52) U.S. Cl. CPC C12N 15/8245; A01H 6/4678; C12N 9/107; C12Y 204/01018

(57) ABSTRACT

A series of independent human-induced non-transgenic mutations found at one or more of the SBEII genes of wheat; wheat plants having these mutations in one or more of their SBEII genes; and a method of creating and finding similar and/or additional mutations of SBEII by screening pooled and/or individual wheat plants. The seeds and flour from the wheat plants of the present invention exhibit an increase in amylose and resistant starch without having the inclusion of foreign nucleic acids in their genomes. Additionally, the wheat plants of the present invention exhibit altered SBEII activity without having the inclusion of foreign nucleic acids in their genomes.

### CROSS-REFERENCE TO RELATED APPLICATIONS

This application is a continuation of U.S. patent application Ser. No. 18/298,041, filed Apr. 10, 2023, which is a continuation of U.S. patent application Ser. No. 17/147,281, filed Jan. 12, 2021, now U.S. Pat. No. 11,649,464 issued May 16, 2023, which is a continuation of U.S. patent application Ser. No. 15/914,994, filed Mar. 7, 2018, now U.S. Pat. No. 10,934,557 issued Mar. 2, 2021, which is a continuation of U.S. patent application Ser. No. 14/825,369, filed Aug. 13, 2015, now U.S. Pat. No. 10,563,217 issued Feb. 18, 2020, which is a continuation of U.S. patent application Ser. No. 13/633,588, filed Oct. 2, 2012, now U.S. Pat. No. 9,150,839, issued Oct. 6, 2015, which claims the benefit of U.S. Provisional Application No. 61/542,953, entitled “WHEAT WITH INCREASED RESISTANT STARCH LEVELS,” filed Oct. 4, 2011; all of which are incorporated herein by reference.

### STATEMENT REGARDING FEDERALLY SPONSORED RESEARCH OR DEVELOPMENT

This invention was made with government support under grant DK085811 awarded by National Institutes of Health. The government has certain rights in the invention.

### SEQUENCE LISTING

The Sequence Listing is being submitted electronically in XML format and is hereby incorporated by reference in its entirety. Said XML copy, created on Apr. 7, 2023, is named 147411_000136_Seq_Listing.xml and is 152,771 bytes in size.

### FIELD

This invention relates to human-induced non-transgenic mutations in one or more starch branching enzyme II (SBEII) genes. In one embodiment, the invention relates to human-induced non-transgenic mutations in one or more SBEII genes of wheat and wheat plants. In still another embodiment, human-induced non-transgenic mutations are in the SBEIIa and/or SBEIIb gene sequences, more particularly, combined mutations in SBEIIa and in both SBEIIa and SBEIIb.

This invention further relates to wheat plants having wheat seeds and wheat flour with increased levels of amylose and increased levels of resistant starch as a result of non-transgenic mutations in at least one of their SBEII genes. This invention also relates to a method that utilizes non-transgenic means to create wheat plants having mutations in at least one of their SBEII genes. In addition, this invention concerns wheat flour and wheat-based food products made from the seeds of these wheat plants having mutations in at least one of their SBEII genes.

### BACKGROUND

An alarming number of adults and children in the United States are either overweight or obese. Healthier food choices, including foods that are high in resistant starch, can help people to better manage their blood sugar levels and their weight. Resistant starch is defined as starch that is not digested in the small intestine of healthy individuals but is fermented in the large intestine. Due to its slow digestion, resistant starch does not have the same caloric load as readily digestible starch, nor does it cause as rapid a rise in blood glucose levels after ingestion. Instead, resistant starch results in a more controlled glucose release over a longer period of time after digestion. This results in a decreased glycemic response, increased insulin sensitivity, and greater feelings of satiety. As a form of dietary fiber, resistant starch contributes to better colon health due to its fermentation by probiotic organisms in the lower gastrointestinal tract into short chain fatty acids, such as butyrate.

In the United States, the majority of dietary starch is consumed in the form of wheat based foods, such as bread, cereals, pastas, and tortillas, which contain very low levels of resistant starch. Cereal starches typically contain less slowly digested amylose (about 25% of total starch) and more highly branched, rapidly digested amylopectin (about 75% of total starch). The amount of amylose in starch positively correlates with the levels of dietary fiber and resistant starch. In corn and barley, loss-of-function mutations of SBEIIb, one of several enzymes in the starch synthesis pathway, have been identified. SBEIIb is the predominant isoform of SBEII expressed in the endosperm of these crops and its loss results in increased amylose and resistant starch levels. In contrast, both SBEIIa and SBEIIb are expressed in the wheat endosperm, but SB Ella is the major isoform that is expressed in this crop. Though there has been great interest in finding mutations that increase amylose content (and therefore resistant starch content) in wheat, wheat lines with increased amylose levels are not commercially available. Preferred mutations would be single nucleotide polymorphisms (SNPs) that reduce or eliminate SBEII enzyme activity (and, in turn, increase amylose levels) without having significant negative pleiotropic effects.

Identification of SNPs in wheat SBEII genes has proceeded slowly because, among other possible reasons, there is limited genetic diversity in today's commercial wheat cultivars and bread wheat is a polyploid, with a complement of 7 chromosomes from each of three ancestors called the A, B and D genomes, resulting in a total of 21 chromosomes. Typically, the bread wheat genome has three functionally redundant copies of each gene (called homoeologs), and therefore, single gene alterations usually do not produce any readily visible phenotype such as those that have been found in diploid corn. Often in wheat, altered variants of all three homoeologs must be combined genetically in order to evaluate their effects. Pasta (durum) wheat is a tetraploid, consisting of A and B genomes, so only two altered copies of each homoeolog must be combined to obtain a phenotype.

To further compound these challenges, SBEIIa and SBEIIb are closely located on the same chromosome in wheat, making it difficult for alleles in these genes to be inherited independently unless through a rare recombination event. Thus, it would be useful to have knock-down or knock-out mutations, resulting from SNPs, of both SBEIIa and SBEIIb of each genome of wheat. The availability of multiple allelic mutations within each SBEII locus, particularly within each SBEII locus of the same genome, would allow for the breeding of new, non-genetically modified wheat lines with a spectrum of increased amylose and resistant starch levels in seeds. Seeds from these lines could be used to produce healthier wheat-based food products, including flour, bread, cereals, pastas, and tortillas.

### SUMMARY

In one embodiment, the invention relates to non-transgenic mutations in one or more SBEII genes. In one embodiment, one or more mutations are in the SBEIIa gene. In another embodiment, one or more mutations are in the SBEIIb gene. In another embodiment, one or more mutations are in each of the SB Ella and SBEIIb genes.

In one embodiment, the invention relates to multiple non-transgenic mutations in the SBEIIa gene including but not limited to 1, 2, 3, 4, 5, 6, 7, 8, 9, 10, and greater than 10 mutations.

In another embodiment, the invention relates to multiple non-transgenic mutations in the SBEIIb gene including but not limited to 1, 2, 3, 4, 5, 6, 7, 8, 9, 10, and greater than 10 mutations.

In another embodiment, the invention relates to multiple non-transgenic mutations in the SBEIIa gene including but not limited to 1, 2, 3, 4, 5, 6, 7, 8, 9, 10, and greater than 10 mutations and multiple mutations in the SBEIIb gene including but not limited to 1, 2, 3, 4, 5, 6, 7, 8, 9, 10, and greater than 10 mutations.

In another embodiment, this invention relates to a wheat plant, wheat seeds, wheat plant parts, and progeny thereof with increased amylose content and increased resistant starch levels compared to wild type wheat plant, wheat seeds, wheat plant parts, and progeny thereof.

In another embodiment, this invention relates to a wheat plant, wheat seeds, wheat plant parts, and progeny thereof having reduced activity of one or more SBEII enzymes compared to the wild type wheat plant, wherein the reduction in SBEII enzyme activity is caused by a human-induced non-transgenic mutation in one or more of the wheat plant's SBEII genes. In another embodiment, the SB Ella enzyme has reduced activity. In yet another embodiment, the SBEIIb enzyme has reduced activity. In still another embodiment, the SBEIIa and SBEIIb enzymes have reduced activity.

In another embodiment, this invention includes a wheat plant containing one or more mutated SBEII genes, as well as seeds, pollen, plant parts and progeny of that plant.

In another embodiment, this invention includes food and food products incorporating wheat seeds and wheat flour having reduced SBEII enzyme activity caused by a human-induced non-transgenic mutation in one or more SBEII genes.

In another embodiment, this invention includes a wheat plant having reduced activity of one or more SBEII enzymes compared to the wild type wheat plants, created by the steps of obtaining plant material from a parent wheat plant, inducing at least one mutation in at least one copy of an SBEII gene of the plant material by treating the plant material with a mutagen to create mutagenized plant material (e.g., seeds or pollen), analyzing progeny wheat plants to detect at least one mutation in at least one copy of a SBEII gene, selecting progeny wheat plants that have at least one mutation in at least one copy of an SBEII gene, crossing progeny wheat plants that have at least one mutation in at least one copy of an SBEII gene with other progeny wheat plants that have at least one mutation in a different copy of an SBEII gene, and repeating the cycle of identifying progeny wheat plants having mutations and crossing the progeny wheat plants having mutations with other progeny wheat plants having mutations to produce progeny wheat plants with reduced SBEII enzyme activity. In another embodiment, the method comprises growing or using the mutagenized plant material to produce progeny wheat plants.

### BRIEF DESCRIPTION OF THE SEQUENCE LISTING

SEQ ID NO: 1 shows a partial Triticum aestivum gene for starch branching enzyme IIa, A genome, exons 1-14.

SEQ ID NO: 2 shows the partial protein sequence encoded by SEQ ID NO: 1.

SEQ ID NO: 3 shows the Triticum aestivum SBEIIa gene for starch branching enzyme IIa, B genome, exons 1-22 (GenBank Accession FM 865435).

SEQ ID NO: 4 shows the protein encoded by SEQ ID NO: 3 (GenBank Accession CAR95900).

SEQ ID NO: 5 shows the Aegilops tauschii gene for starch branching enzyme IIa, D genome, complete sequence exons 1-22 (GenBank Accession A F338431).

SEQ ID NO: 6 shows the protein encoded by SEQ ID NO: 5 (GenBank Accession AAK26821).

SEQ ID NO: 7 shows a partial Triticum aestivum gene for starch branching enzyme IIb, A genome, exons 1-11.

SEQ ID NO: 8 shows the partial protein encoded by SEQ ID NO: 7.

SEQ ID NO: 9 shows the partial Triticum aestivum gene for starch branching enzyme IIb, B genome, exons 1-11.

SEQ ID NO: 10 shows the partial protein encoded by SEQ ID NO: 9.

SEQ ID NO: 11 shows the partial Aegilops tauschii gene for starch branching enzyme IIb, D genome, exons 1-16 (GenBank Accession AY 740398).

SEQ ID NO: 12 shows the partial protein encoded by SEQ ID NO: 11 (GenBank Accession AAW80632).

SEQ ID NOs: 13-58 show exemplary homoeolog specific primers that have proven useful in identifying useful mutations within the SBEIIa and SBEIIb gene sequences.

### DETAILED DESCRIPTION

### Definitions

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

As used herein, the term “allele” is any of one or more alternative forms of a gene, all of which relate to one trait or characteristic. In a diploid cell or organism, the two alleles of a given gene occupy corresponding loci on a pair of homologous chromosomes.

As used herein, amino acid or nucleotide sequence “identity” and “similarity” are determined from an optimal global alignment between the two sequences being compared. A n optimal global alignment is achieved using, for example, the Needleman-Wunsch algorithm (Needleman and Wunsch, 1970, J. Mol. Biol. 48:443-453). Sequences may also be aligned using algorithms known in the art including but not limited to CLUSTAL V algorithm or the Blastn or BLAST 2 sequence programs.

“Identity” means that an amino acid or nucleotide at a particular position in a first polypeptide or polynucleotide is identical to a corresponding amino acid or nucleotide in a second polypeptide or polynucleotide that is in an optimal global alignment with the first polypeptide or polynucleotide. In contrast to identity, “similarity” encompasses amino acids that are conservative substitutions. A “conservative” substitution is any substitution that has a positive score in the Blosum62 substitution matrix (Hentikoff and Hentikoff, 1992, Proc. Natl. Acad. Sci. USA 89: 10915-10919).

By the statement “sequence A is n % similar to sequence B,” it is meant that n % of the positions of an optimal global alignment between sequences A and B consists of identical residues or nucleotides and conservative substitutions. By the statement “sequence A is n % identical to sequence B,” it is meant that n % of the positions of an optimal global alignment between sequences A and B consists of identical residues or nucleotides.

As used herein, the term “plant” includes reference to an immature or mature whole plant, including a plant from which seed or grain or anthers have been removed. A seed or embryo that will produce the plant is also considered to be the plant.

As used herein, the term “plant parts” includes plant protoplasts, plant cell tissue cultures from which wheat plants can be regenerated, plant calli, plant clumps, and plant cells that are intact in plants or parts of plants, such as embryos, pollen, ovules, pericarp, seed, flowers, florets, heads, spikes, leaves, roots, root tips, anthers, and the like.

As used herein, the term “polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers, and to longer chains generally referred to as proteins. Polypeptides may contain amino acids other than the 20 gene-encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature and they are well known to those of skill in the art. It will be appreciated that the same type of modification may be present in the same or varying degree at several sites in a given polypeptide.

A s used herein, an “SBEII derivative” refers to a SBEII protein/peptide/polypeptide sequence that possesses biological activity that is substantially reduced as compared to the biological activity of the whole SBEII protein/peptide/polypeptide sequence. In other words, it refers to a polypeptide of a modified SBEII protein of the invention that has reduced SBEII enzymatic activity. The term “SBEII derivative” encompasses the “fragments” or “chemical derivatives” of a modified SBEII protein/peptide.

As used herein, the term “polynucleotide(s)” generally refers to any polyribonucleotide or poly-deoxyribonucleotide, which may be unmodified RNA or DNA or modified RNA or DNA. This definition includes, without limitation, single- and double-stranded DNA, DNA that is a mixture of single- and double-stranded regions or single-, double- and triple-stranded regions, cDNA, single- and double-stranded RNA, and RNA that is a mixture of single- and double-stranded regions, hybrid molecules comprising DNA and RNA that may be single-stranded or, more typically, double-stranded, or triple-stranded regions, or a mixture of single- and double-stranded regions. The term “polynucleotide(s)” also embraces short nucleotides or fragments, often referred to as “oligonucleotides,” that due to mutagenesis are not 100% identical but nevertheless code for the same amino acid sequence.

A “reduced or non-functional fragment,” as is used herein, refers to a nucleic acid sequence that encodes for a SBEII protein that has reduced biological activity as compared the protein coding of the whole nucleic acid sequence. In other words, it refers to a nucleic acid or fragment(s) thereof that substantially retains the capacity of encoding an SBEII polypeptide of the invention, but the encoded SBEII polypeptide has reduced activity.

The term “fragment,” as used herein, refers to a polynucleotide sequence, (e.g, a PCR fragment) which is an isolated portion of the subject nucleic acid constructed artificially (e.g., by chemical synthesis) or by cleaving a natural product into multiple pieces, using restriction endonucleases or mechanical shearing, or a portion of a nucleic acid synthesized by PCR, DNA polymerase or any other polymerizing technique well known in the art, or expressed in a host cell by recombinant nucleic acid technology well known to one of skill in the art.

With reference to polynucleotides of the invention, the term “isolated polynucleotide” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated polynucleotide” may comprise a PCR fragment. In another embodiment, the “isolated polynucleotide” may comprise a DNA molecule inserted into a vector, such as a plasmid or virus vector, or integrated into the genomic DNA of a prokaryote or eukaryote. An “isolated polynucleotide molecule” may also comprise a cDNA molecule.

In one embodiment, the invention relates to non-transgenic mutations in one or more SBEII genes. In another embodiment, the invention describes wheat plants exhibiting seeds with increased amylose content and increased resistant starch levels compared to wild type wheat seeds, without the inclusion of foreign nucleic acids in the wheat plants' genomes.

In still another embodiment, the invention relates to a series of independent human-induced non-transgenic mutations in one or more SBEII genes; wheat plants having one or more of these mutations in at least one SBEII gene thereof; and a method of creating and identifying similar and/or additional mutations in at least one SBEII gene of wheat. Additionally, the invention relates to wheat plants exhibiting seed with increased amylose and resistant starch content compared to wild type wheat seed, without the inclusion of foreign nucleic acids in the plants' genomes.

### SBEII Mutations

### A. SBEII Genes

In one embodiment, the invention relates to one or more non-transgenic mutations in the SBEII gene. In another embodiment, the SBEII gene may contain one or more non-transgenic mutations recited in Tables 1-6 and 8-12 and corresponding mutations in homoeologues and combinations thereof.

In another embodiment, the invention comprises corresponding mutations to the one or more non-transgenic mutations disclosed herein in the SBEII gene in a corresponding homoeologue. By way of example, an identified mutation in the SBEIIa gene of the A genome may be a beneficial mutation in the SBEIIa gene of the B and/or D genome. One of ordinary skill in the art will understand that the mutation in the homoeologue may not be in the exact location.

One of ordinary skill in the art understands there is natural variation in the genetic sequences of the SBEII genes in different wheat varieties. The degree of sequence identity between homologous SBEIIa genes or the proteins is believed to be about 90%. This is true for SBEIIb genes and proteins as well.

The inventors have determined that to achieve a high amylose phenotype in wheat plants, mutations that reduce SBEII gene function are desirable. Preferred mutations include missense and nonsense changes, including mutations that prematurely truncate the translation of one or more SBEII proteins from messenger RNA, such as those mutations that create a stop codon within the coding region of an SBEII messenger RNA. Such mutations include insertions, repeat sequences, splice junction mutations, modified open reading frames (ORFs) and point mutations.

### 1. SBEIIa Genes

In another embodiment, the invention relates to one or more mutations in the SBEIIa gene. In one embodiment, the invention relates to multiple non-transgenic mutations in the SBEIIa gene including but not limited to 1, 2, 3, 4, 5, 6, 7, 8, 9, 10, and greater than 10 mutations.

In still another embodiment, one or more mutations are in the SBEIIa gene of the A genome. In another embodiment, one or more mutations are in the SBEIIa gene of the B genome. In still another embodiment, one or more mutations are in the SBEIIa gene of the D genome. In yet another embodiment, one or more mutations are in the SB Ella genes of the A and B genomes. In still another embodiment, one or more mutations are in the SB Ella genes of the A and D genomes. In another embodiment, one or more mutations are in the SBEIIa genes of the B and D genomes. In yet another embodiment, one or more mutations are in the SBEIIa genes of the A, B, and D genomes.

In one embodiment, one or more non-transgenic mutations are in both alleles of the SBEIIa gene in the A genome. In another embodiment, the non-transgenic mutations are identical in both alleles of the SBEIIa gene of the A genome.

In one embodiment, one or more non-transgenic mutations are in both alleles of the SBEIIa gene in the B genome. In another embodiment, the non-transgenic mutations are identical in both alleles of the SBEIIa gene of the B genome.

In one embodiment, one or more non-transgenic mutations are in both alleles of the SBEIIa gene in the D genome. In another embodiment, the non-transgenic mutations are identical in both alleles of the SBEIIa gene of the D genome.

The following mutations are exemplary of the mutations created and identified according to various embodiments of the invention. SEQ ID NOs 1-6 are reference sequences for SBEIIa. SEQ ID NOs 7-12 are reference sequences for SBEIIb.

The following mutations identified in Tables 1-6 are exemplary of the mutations created and identified according to various embodiments of the invention. They are offered by way of illustration, not limitation. It is to be understood that the mutations below are merely exemplary and that similar mutations are also contemplated.

The nomenclature used in Tables 1-6 and 8-12 indicates the wild type nucleotide or amino acid, followed by its position according to the referenced sequence, followed by the changed nucleotide or amino acid (A.A.) at that position using standard genetic code terminology. A n asterisk is used to designate a stop codon, also called a truncation mutation.

One exemplary mutation is G5267A, resulting in a change from guanine to adenine at nucleotide position 5267 identified according to its position in the sequence of SEQ ID NO: 1. This mutation results in a change from tryptophan to a stop mutation at amino acid position 436 identified according to its position in the expressed protein (SEQ ID NO: 2).

TABLE 1
Examples of mutations created and identified in SBEIIa
in the A genome of wheat plants. Nucleotide and amino
acid changes are identified according to their positions
in SEQ ID NOs: 1 and 2, respectively.

| | Primer SEQ | Nucleotide | A.A. | | |
|---|---|---|---|---|---|
| Variety | IDs. | Mutation | Mutation | PSSM | SIFT |
| Express | 13, 14 | C538T | V51= | | |
| Express | 13, 14 | G586A | E67= | | |
| Express | 13, 14 | C605T | P74S | | 0.89 |
| Express | 13, 14 | G608A | A75T | | 0.67 |
| Express | 13, 14 | C644T | Intron | | |
| Express | 13, 14 | G648A | Intron | | |
| Express | 13, 14 | C853T | Intron | | |
| Express | 13, 14 | G951A | G97= | | |
| Express | 13, 14 | G952A | G98R | | 0.44 |
| Express | 13, 14 | G1036A | E126K | | 0.86 |
| Express | 13, 14 | G1059A | P133= | | |
| Express | 15, 16 | C2384T | Intron | | |
| Express | 15, 16 | C2384T | Intron | | |
| Express | 15, 16 | C2394T | Intron | | |
| Express | 15, 16 | G2574A | Intron | | |
| Express | 15, 16 | G2582A | Splice | | |
| | | | Junction | | |
| Express | 15, 16 | G2592A | D260N | 10.4 | 0.3 |
| Express | 15, 16 | G2605A | G264D | 22 | 0 |
| Express | 15, 16 | G2612A | K266= | | |
| Express | 15, 16 | G2625A | A271T | 10.8 | 0.04 |
| Express | 15, 16 | C2664T | P284S | 20.3 | 0.01 |
| Express | 15, 16 | G2674A | G287D | 19.4 | 0 |
| Express | 15, 16 | C2857T | Intron | | |
| Express | 15, 16 | C2861T | Intron | | |
| Express | 15, 16 | C2921T | Intron | | |
| Express | 15, 16 | G2990A | E296K | | 0.03 |
| Express | 15, 16 | C3004T | F300= | | |
| Express | 15, 16 | G3039A | R312K | 8.2 | 0.08 |
| Express | 15, 16 | A3155T | Intron | | |
| Express | 17, 18 | C5164T | Intron | | |
| Express | 17, 18 | C5164T | Intron | | |
| Express | 17, 18 | G5196A | G413S | 13.8 | 0 |
| Kronos | 17, 18 | G5239A | G427D | 6.6 | 0.09 |
| Kronos | 17, 18 | C5256T | H433Y | 22.3 | 0 |
| Express | 17, 18 | G5267A | W436* | | |
| Kronos | 17, 18 | G5267A | W436* | | |
| Express | 17, 18 | G5268A | D437N | 7.9 | 0.04 |
| Express | 17, 18 | G5268A | D437N | 7.9 | 0.04 |
| Kronos | 17, 18 | G5268A | D437N | 7.9 | 0.04 |
| Express | 17, 18 | G5289A | G444R | 19 | 0 |
| Kronos | 17, 18 | G5289A | G444R | 19 | 0 |
| Express | 17, 18 | G5298A | E447K | 8.9 | 0.02 |
| Express | 17, 18 | G5301A | Splice | | |
| | | | Junction | | |
| Express | 17, 18 | G5301A | Splice | | |
| | | | Junction | | |
| Express | 17, 18 | G5305A | Intron | | |
| Kronos | 17, 18 | G5308A | Intron | | |
| Express | 17, 18 | C5315T | Intron | | |
| Express | 17, 18 | C5315T | Intron | | |
| Express | 17, 18 | C5315T | Intron | | |
| Express | 17, 18 | C5324T | Intron | | |
| Kronos | 17, 18 | C5325T | Intron | | |
| Kronos | 17, 18 | G5332A | Intron | | |
| Express | 17, 18 | G5386A | Intron | | |
| Express | 17, 18 | C5405T | L453= | | |
| Express | 17, 18 | C5405T | L453= | | |
| Express | 17, 18 | G5418A | R457K | 18.3 | 0.01 |
| Express | 17, 18 | G5422A | W458* | | |
| Kronos | 17, 18 | G5429A | E461K | 17.1 | 0.01 |
| Kronos | 17, 18 | G5429A | E461K | 17.1 | 0.01 |
| Express | 17, 18 | G5432A | E462K | 17.6 | 0.01 |
| Express | 17, 18 | G5432A | E462K | 17.6 | 0.01 |
| Express | 17, 18 | G5448A | G467E | 27.1 | 0 |
| Express | 17, 18 | G5463A | G472E | 27.1 | 0 |
| Express | 17, 18 | G5463A | G472E | 27.1 | 0 |
| Express | 17, 18 | G5463A | G472E | 27.1 | 0 |
| Express | 17, 18 | G5464A | G472= | | |
| Express | 17, 18 | G5465A | V473M | 17.1 | 0 |
| Express | 17, 18 | C5470T | T474= | | |
| Kronos | 17, 18 | C5470T | T474= | | |
| Express | 17, 18 | C5484T | T479I | 10.3 | 0.4 |
| Kronos | 17, 18 | G5493A | G482E | 27.1 | 0 |
| Kronos | 17, 18 | G5522A | Intron | | |
| Express | 17, 18 | G5534A | Intron | | |
| Express | 17, 18 | G5655A | Intron | | |
| Express | 17, 18 | C5712T | T488I | 16.9 | 0 |
| Express | 17, 18 | C5712T | T488I | 16.9 | 0 |
| Express | 17, 18 | C5719T | N490= | | |
| Express | 17, 18 | G5736A | G496E | 22.1 | 0 |
| Express | 17, 18 | C5745T | T499I | 15.8 | 0.02 |
| Express | 17, 18 | G5753A | D502N | 17.1 | 0.01 |
| Express | 17, 18 | G5756A | A503T | 19.8 | 0 |
| Express | 17, 18 | C5757T | A503V | 19.2 | 0 |
| Express | 17, 18 | G5783A | D512N | 7.8 | 0.18 |
| Kronos | 17, 18 | C5801T | H518Y | −8.3 | 1 |
| Express | 17, 18 | C5804T | P519S | 26.7 | 0 |
| Express | 17, 18 | C5811T | A521V | 6.3 | 0.21 |
| Express | 17, 18 | C5811T | A521V | 6.3 | 0.21 |
| Express | 17, 18 | G5831A | Splice | | |
| | | | Junction | | |
| Express | 17, 18 | G5852A | Intron | | |
| Express | 17, 18 | C5921T | Intron | | |
| Express | 17, 18 | G5956A | Intron | | |
| Express | 17, 18 | G5956A | Intron | | |

In one embodiment, the invention relates to a polynucleotide of the SB Ella gene in the A genome with one or more non-transgenic mutations listed in Table 1 and corresponding to SEQ ID NO: 1. In another embodiment, the polynucleotide with one or more non-transgenic mutations listed in Table 1 is 85%, 86%, 87%, 88%, 89%, 90%, 91%, 92%, 93%, 94%, 95%, 96%, 97%, 98%, 99% or greater than 99% identical to SEQ ID NO: 1. In yet another embodiment, the polynucleotide with one or more non-transgenic mutations listed in Table 1 is 85%, 86%, 87%, 88%, 89%, 90%, 91%, 92%, 93%, 94%, 95%, 96%, 97%, 98%, 99% or greater than 99% similar to SEQ ID NO: 1.

In still another embodiment, the polynucleotide with one or more non-transgenic mutation listed in Table 1 codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical to SEQ ID NO: 2. In still another embodiment, the polynucleotide with one or more non-transgenic mutation listed in Table 1 codes for a SB Ella protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% similar to SEQ ID NO: 2.

Examples of mutations created and identified in SBEIIa in the B genome of wheat plants are provided in Table 2. Nucleotide and amino acid changes are identified according to their positions in SEQ ID NOs: 3 and 4, respectively.

TABLE 2
Representative mutations in the SBEIIa gene in the B genome

| | Primer SEQ | Nucleotide | A.A. | | |
|---|---|---|---|---|---|
| Variety | IDs. | Mutation | Mutation | PSSM | SIFT |
| Express | 23, 24 | C4792T | Intron | | |
| Express | 23, 24 | G4830A | Intron | | |
| Express | 23, 24 | C4878T | Intron | | |
| Kronos | 23, 24 | C4881T | Intron | | |
| Express | 23, 24 | C4937T | Intron | | |
| Express | 23, 24 | C4960T | T410I | 4.8 | 0.25 |
| Express | 23, 24 | C4960A | T410N | 13.9 | 0.02 |
| Express | 23, 24 | C4961T | T410= | | |
| Express | 23, 24 | G4978A | G416D | 14.5 | 0.73 |
| Express | 23, 24 | G4987A | G419D | 16.8 | 0.01 |
| Express | 23, 24 | G4987A | G419D | 16.8 | 0.01 |
| Express | 23, 24 | C4990T | T420I | 21.4 | 0 |
| Express | 23, 24 | C4998T | H423Y | 15.5 | 0.59 |
| Express | 23, 24 | C5006T | F425= | | |
| Kronos | 23, 24 | G5011A | G427D | −0.4 | 0.5 |
| Express | 23, 24 | C5017T | P429L | 14.1 | 0.11 |
| Express | 23, 24 | G5020A | R430H | 21.4 | 0 |
| Kronos | 23, 24 | G5020A | R430H | 21.4 | 0 |
| Kronos | 23, 24 | G5020A | R430H | 21.4 | 0 |
| Kronos | 23, 24 | G5020A | R430H | 21.4 | 0 |
| Kronos | 23, 24 | G5022A | G431S | 25.2 | 0 |
| Kronos | 23, 24 | C5025T | H432Y | −3.6 | 1 |
| Express | 23, 24 | G5032A | W434* | | |
| Kronos | 23, 24 | G5033A | W434* | | |
| Express | 23, 24 | G5036A | M435I | 15 | 0.03 |
| Express | 23, 24 | G5038A | W436* | | |
| Express | 23, 24 | G5038A | W436* | | |
| Kronos | 23, 24 | G5040A | D437N | 19.9 | 0.01 |
| Express | 23, 24 | G5040A | D437N | 19.9 | 0.01 |
| Express | 23, 24 | C5044T | S438F | 12.1 | 0.01 |
| Express | 23, 24 | G5062A | G444E | 17 | 0 |
| Kronos | 23, 24 | G5062A | G444E | 17 | 0 |
| Kronos | 23, 24 | G5062A | G444E | 17 | 0 |
| Kronos | 23, 24 | G5063A | G444= | | |
| Kronos | 23, 24 | G5065A | S445N | −4.7 | 1 |
| Express | 23, 24 | G5068A | W446* | | |
| Express | 23, 24 | G5069A | W446* | | |
| Express | 23, 24 | G5069A | W446* | | |
| Kronos | 23, 24 | G5069A | W446* | | |
| Express | 23, 24 | G5069A | W446* | | |
| Express | 23, 24 | G5069A | W446* | | |
| Express | 23, 24 | G5069A | W446* | | |
| Express | 23, 24 | G5070A | E447K | 9.3 | 0.02 |
| Express | 23, 24 | G5070A | E447K | 9.3 | 0.02 |
| Kronos | 23, 24 | G5073A | Splice | | |
| | | | Junction | | |
| Kronos | 23, 24 | G5080A | Intron | | |
| Express | 23, 24 | C5081T | Intron | | |
| Express | 23, 24 | G5083A | Intron | | |
| Kronos | 23, 24 | C5087T | Intron | | |
| Express | 23, 24 | C5090T | Intron | | |
| Kronos | 23, 24 | C5090T | Intron | | |
| Kronos | 23, 24 | C5090T | Intron | | |
| Express | 23, 24 | C5090T | Intron | | |
| Express | 23, 24 | G5092A | Intron | | |
| Kronos | 23, 24 | G5105A | Intron | | |
| Express | 23, 24 | G5112A | Intron | | |
| Kronos | 23, 24 | G5112A | Intron | | |
| Kronos | 23, 24 | C5129T | Intron | | |
| Kronos | 23, 24 | C5129T | Intron | | |
| Express | 23, 24 | C5158T | Intron | | |
| Express | 23, 24 | G5160A | Splice | | |
| | | | Junction | | |
| Express | 23, 24 | G5161A | V448I | | 0.01 |
| Express | 23, 24 | G5161A | V448I | | 0.01 |
| Express | 23, 24 | G5161A | V448I | | 0.01 |
| Express | 23, 24 | G5168A | R450K | 19 | 0.01 |
| Express | 23, 24 | G5168A | R450K | 19 | 0.01 |
| Kronos | 23, 24 | G5168A | R450K | 19 | 0.01 |
| Express | 23, 24 | C5172T | F451= | | |
| Express | 23, 24 | G5185A | A456T | 13.3 | 0.11 |
| Express | 23, 24 | G5185A | A456T | 13.3 | 0.11 |
| Kronos | 23, 24 | G5189A | R457K | 19 | 0.01 |
| Express | 23, 24 | G5193A | W458* | | |
| Express | 23, 24 | C5197T | L460F | 11.7 | 0.02 |
| Express | 23, 24 | G5200A | E461K | 18.3 | 0.01 |
| Kronos | 23, 24 | G5203A | E462K | 18.3 | 0 |
| Express | 23, 24 | G5203A | E462K | 18.3 | 0 |
| Kronos | 23, 24 | G5211A | K464= | | |
| Kronos | 23, 24 | G5211A | K464= | | |
| Express | 23, 24 | G5219A | G467E | 27.7 | 0 |
| Kronos | 23, 24 | G5219A | G467E | 27.7 | 0 |
| Kronos | 23, 24 | G5219A | G467E | 27.7 | 0 |
| Kronos | 23, 24 | G5219A | G467E | 27.7 | 0 |
| Kronos | 23, 24 | T5223C | F468= | | |
| Express | 23, 24 | C5224T | R469* | | |
| Kronos | 23, 24 | G5233A | G472R | 27.3 | 0 |
| Kronos | 23, 24 | G5234A | G472E | 27.7 | 0 |
| Kronos | 23, 24 | G5234A | G472E | 27.7 | 0 |
| Express | 23, 24 | G5234A | G472E | 27.7 | 0 |
| Kronos | 23, 24 | C5240T | T474I | 21.9 | 0 |
| Kronos | 23, 24 | C5244T | S475= | | |
| Express | 23, 24 | C5255T | T479I | 9.8 | 0.55 |
| Express | 23, 24 | G5264A | G482E | 27.7 | 0 |
| Express | 23, 24 | G5272A | Splice | | |
| | | | Junction | | |
| Express | 23, 24 | G5272A | Splice | | |
| | | | Junction | | |
| Kronos | 23, 24 | G5272A | Splice | | |
| | | | Junction | | |
| Kronos | 23, 24 | G5276A | Intron | | |
| Express | 23, 24 | G5284A | Intron | | |
| Express | 23, 24 | G5286A | Intron | | |
| Express | 23, 24 | G5287A | Intron | | |
| Kronos | 23, 24 | G5287A | Intron | | |
| Kronos | 23, 24 | C5297T | Intron | | |
| Kronos | 23, 24 | C5297T | Intron | | |
| Kronos | 23, 24 | G5306A | Intron | | |
| Express | 23, 24 | C5330T | Intron | | |
| Express | 23, 24 | G5338A | Intron | | |
| Express | 23, 24 | G5350A | Intron | | |
| Express | 23, 24 | G5350A | Intron | | |
| Express | 23, 24 | C5353T | Intron | | |
| Express | 23, 24 | G5364A | Intron | | |
| Express | 23, 24 | G5364A | Intron | | |
| Express | 23, 24 | G5372A | Intron | | |
| Express | 23, 24 | G5372A | Intron | | |
| Express | 23, 24 | C5379T | Intron | | |
| Express | 23, 24 | C5395T | Intron | | |
| Express | 23, 24 | G5409A | Intron | | |
| Express | 23, 24 | G5421A | Intron | | |
| Express | 23, 24 | C5448T | Intron | | |
| Express | 23, 24 | T5450C | Intron | | |
| Kronos | 23, 24 | C5469T | Intron | | |
| Express | 23, 24 | G5472A | Splice | | |
| | | | Junction | | |
| Express | 23, 24 | G5475A | M485I | | 0.18 |
| Express | 23, 24 | G5495A | G492D | −0.8 | 0.39 |
| Express | 23, 24 | T5522A | V501D | 8.3 | 0.08 |
| Express | 23, 24 | C5528A | A503E | 19.9 | 0 |
| Express | 23, 24 | G5530A | V504M | 7.8 | 0.04 |
| Express | 23, 24 | C5553T | N511= | | |
| Express | 23, 24 | G5566A | G516R | 5.2 | 0.32 |
| Express | 23, 24 | C5575T | P519S | 17.4 | 0.02 |
| Kronos | 23, 24 | C5582T | A521V | 4.8 | 0.33 |
| Kronos | 23, 24 | C5582T | A521V | 4.8 | 0.33 |
| Express | 23, 24 | C5589T | S523= | | |
| Express | 23, 24 | G5606A | Intron | | |
| Express | 23, 24 | G5646A | Intron | | |
| Express | 23, 24 | C5662T | Intron | | |
| Express | 23, 24 | C5662T | Intron | | |
| Express | 23, 24 | G5675A | Intron | | |
| Express | 23, 24 | G5675A | Intron | | |
| Express | 23, 24 | G5835A | Intron | | |
| Express | 23, 24 | C4960T | T410I | 4.8 | 0.25 |
| Express | 23, 24 | G4987A | G419D | 16.8 | 0.01 |
| Express | 23, 24 | G5185A | A456T | 13.3 | 0.11 |
| Express | 23, 24 | C5243T | S475F | 26.4 | 0 |
| Express | 23, 24 | C5255T | T479I | 9.8 | 0.55 |
| Express | 21, 22 | G2386A | G233D | | 0 |
| Express | 21, 22 | G2456A | K256= | | |
| Express | 21, 22 | G2464A | Intron | | |
| Express | 21, 22 | G2483A | Intron | | |
| Express | 21, 22 | C2509T | Intron | | |
| Express | 21, 22 | C2518T | Intron | | |
| Express | 21, 22 | G2606A | A279T | 3.1 | 0.14 |
| Express | 21, 22 | C2610T | P280L | 5.1 | 0.47 |
| Express | 21, 22 | G2613A | G281D | 2.7 | 0.36 |
| Express | 21, 22 | G2613A | G281D | 2.7 | 0.36 |
| Express | 21, 22 | C2648T | P293S | | 0.08 |
| Express | 21, 22 | G2661A | Intron | | |
| Express | 21, 22 | G2661A | Intron | | |
| Express | 21, 22 | G2689A | Intron | | |
| Express | 21, 22 | G2945A | Splice | | |
| | | | Junction | | |
| Express | 21, 22 | C2967T | P303S | 8.4 | 0.17 |
| Express | 21, 22 | C2967T | P303S | 8.4 | 0.17 |
| Express | 21, 22 | G2456A | K256= | | |
| Express | 21, 22 | C2518T | Intron | | |
| Express | 21, 22 | G2606A | A279T | 3.1 | 0.14 |
| Express | 21, 22 | G2606A | A279T | 3.1 | 0.14 |
| Express | 21, 22 | C2648T | P293S | | 0.08 |
| Express | 21, 22 | G2661A | Intron | | |
| Express | 21, 22 | C2967T | P303S | 8.4 | 0.17 |

In one embodiment, the invention relates to a polynucleotide of the SB Ella gene in the B genome with one or more non-transgenic mutations listed in Table 2 and corresponding to SEQ ID NO: 3. In another embodiment, the polynucleotide with one or more non-transgenic mutations listed in Table 2 is 85%, 86%, 87%, 88%, 89%, 90%, 91%, 92%, 93%, 94%, 95%, 96%, 97%, 98%, 99% or greater than 99% identical to SEQ ID NO: 3. In yet another embodiment, the polynucleotide with one or more non-transgenic mutations listed in Table 2 is 85%, 86%, 87%, 88%, 89%, 90%, 91%, 92%, 93%, 94%, 95%, 96%, 97%, 98%, 99% or greater than 99% similar to SEQ ID NO: 3.

In still another embodiment, the polynucleotide with one or more non-transgenic mutation listed in Table 2 codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical to SEQ ID NO: 4. In still another embodiment, the polynucleotide with one or more non-transgenic mutations listed in Table 2 codes for a SB Ella protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% similar to SEQ ID NO: 4.

Examples of mutations created and identified in SBEIIa in the D genome of wheat plants are provided in Table 3. Nucleotide and amino acid changes are identified according to their positions in SEQ ID NOs: 5 and 6, respectively.

TABLE 3
Representative mutations in SBEIIa gene in the D genome

| | Primer SEQ | Nucleotide | A.A. | | |
|---|---|---|---|---|---|
| Variety | IDs. | Mutation | Mutation | PSSM | SIFT |
| Express | 25, 26 | C1708T | P60S | 13.4 | 0.03 |
| Express | 25, 26 | G1721A | S64N | −16.8 | 0.76 |
| Express | 25, 26 | G1753A | E75K | | 0.74 |
| Express | 25, 26 | G1753A | E75K | | 0.74 |
| Express | 25, 26 | G1761A | Q77= | | |
| Express | 25, 26 | G1761A | Q77= | | |
| Express | 25, 26 | G1762A | Splice | | |
| | | | Junction | | |
| Express | 25, 26 | G1762A | Splice | | |
| | | | Junction | | |
| Express | 25, 26 | G1780A | Intron | | |
| Express | 25, 26 | G1962A | Intron | | |
| Express | 25, 26 | G2037A | Splice | | |
| | | | Junction | | |
| Express | 25, 26 | G1962A | Intron | | |
| Express | 25, 26 | G2037A | Splice | | |
| | | | Junction | | |
| Express | 25, 26 | C1999T | Intron | | |
| Express | 25, 26 | G2185A | E127K | | 0.79 |
| Express | 25, 26 | C1999T | Intron | | |
| Express | 25, 26 | C2011T | Intron | | |
| Express | 25, 26 | C2028T | Intron | | |
| Express | 25, 26 | C2028T | Intron | | |
| Express | 25, 26 | C2032T | Intron | | |
| Express | 25, 26 | G2065A | A87T | | 0.59 |
| Express | 25, 26 | G2065A | A87T | | 0.59 |
| Express | 25, 26 | G2065A | A87T | | 0.59 |
| Express | 25, 26 | G2079A | M91I | | 0.76 |
| Express | 25, 26 | G2086A | G94R | | 0.15 |
| Express | 25, 26 | G2087A | G94E | | 0.43 |
| Express | 25, 26 | G2126A | G107D | | 0.53 |
| Express | 25, 26 | G2131A | V109M | | 0.14 |
| Express | 25, 26 | G2134A | E110K | | 0.64 |
| Express | 25, 26 | G2149A | G115S | | 0.37 |
| Express | 25, 26 | G2149A | G115S | | 0.37 |
| Express | 25, 26 | G2183A | G126E | | 1 |
| Express | 25, 26 | G2187A | E127= | | |
| Express | 25, 26 | G2220A | G138= | | |
| Express | 25, 26 | C2266T | H154Y | 16.9 | 0.03 |
| Express | 25, 26 | C2286T | Intron | | |
| Express | 25, 26 | C2303T | Intron | | |
| Express | 27, 28 | C3589T | S242= | | |
| Express | 27, 28 | C3602T | H247Y | 23.2 | 0 |
| Express | 27, 28 | C3607A | G248= | | |
| Express | 27, 28 | C3611G | R250G | 16 | 0.01 |
| Express | 27, 28 | G3649A | Intron | | |
| Express | 27, 28 | G3677A | Intron | | |
| Express | 27, 28 | G3677A | Intron | | |
| Express | 27, 28 | C3743T | S266F | 16.9 | 0 |
| Express | 27, 28 | C3753T | I269= | | |
| Express | 27, 28 | C3772T | P276S | 9.5 | 0.35 |
| Express | 27, 28 | G3793A | G283S | 10.9 | 0.08 |
| Express | 27, 28 | G3794A | G283D | 16.3 | 0.01 |
| Express | 27, 28 | G3824A | Intron | | |
| Express | 27, 28 | G4083A | Intron | | |
| Express | 27, 28 | C4119T | F296= | | |
| Express | 27, 28 | C4126T | P299S | 9 | 0.15 |
| Express | 27, 28 | C4127T | P299L | 18.1 | 0.01 |
| Express | 29, 30 | G4818A | E320K | 7.9 | 0.11 |
| Express | 29, 30 | G4839A | A327T | 9.2 | 0.24 |
| Express | 29, 30 | G4850A | R330= | | |
| Express | 29, 30 | G4850A | R330= | | |
| Express | 29, 30 | G4851A | D331N | 13 | 0.02 |
| Express | 29, 30 | G4939A | G360E | 24.5 | 0 |
| Express | 29, 30 | C5118T | Y361= | | |
| Express | 29, 30 | G5144A | S370N | 22.9 | 0 |
| Express | 29, 30 | G5156A | G374E | 24.5 | 0 |
| Express | 29, 30 | G5156A | G374E | 24.5 | 0 |
| Express | 29, 30 | G5166A | E377= | | |
| Express | 29, 30 | C5169T | D378= | | |
| Express | 29, 30 | G5204A | G390D | 22.8 | 0 |
| Express | 29, 30 | G5258A | Intron | | |
| Express | 29, 30 | C5267T | Intron | | |
| Express | 29, 30 | C5275T | Intron | | |
| Express | 29, 30 | G5299A | Intron | | |
| Express | 31, 32 | G6793A | A499T | 18.7 | 0 |
| Express | 31, 32 | C6163T | Intron | | |
| Express | 31, 32 | G6793A | A499T | 18.7 | 0 |
| Express | 31, 32 | C6163T | Intron | | |
| Express | 31, 32 | G6793A | A499T | 18.7 | 0 |
| Express | 31, 32 | C6163T | Intron | | |
| Express | 31, 32 | G6174A | Intron | | |
| Express | 31, 32 | C6183T | Intron | | |
| Express | 31, 32 | C6227T | T406= | | |
| Express | 31, 32 | G6258A | D417N | 6.8 | 0.15 |
| Express | 31, 32 | G6258A | D417N | 6.8 | 0.15 |
| Express | 31, 32 | C6275T | H422= | | |
| Express | 31, 32 | G6277A | G423D | 0.6 | 0.45 |
| Express | 31, 32 | G6277A | G423D | 0.6 | 0.45 |
| Express | 31, 32 | G6286A | R426H | 21.5 | 0 |
| Express | 31, 32 | G6286A | R426H | 21.5 | 0 |
| Express | 31, 32 | G6305A | W432* | | |
| Express | 31, 32 | G6306A | D433N | 20.1 | 0.01 |
| Express | 31, 32 | G6306A | D433N | 20.1 | 0.01 |
| Express | 31, 32 | C6320T | F437= | | |
| Express | 31, 32 | G6327A | G440R | 17.2 | 0 |
| Express | 31, 32 | G6328A | G440E | 17.3 | 0 |
| Express | 31, 32 | G6329A | G440= | | |
| Express | 31, 32 | G6335A | W442* | | |
| Express | 31, 32 | G6336A | E443K | 9.4 | 0.02 |
| Express | 31, 32 | C6418T | Intron | | |
| Express | 31, 32 | G6426A | Splice | | |
| | | | Junction | | |
| Express | 31, 32 | C6442T | L449= | | |
| Express | 31, 32 | C6442T | L449= | | |
| Express | 31, 32 | G6451A | A452T | 13.2 | 0.08 |
| Express | 31, 32 | G6459A | W454* | | |
| Express | 31, 32 | C6463T | L456F | 11.6 | 0.02 |
| Express | 31, 32 | G6496A | D467N | 23.2 | 0 |
| Express | 31, 32 | C6525T | H476= | | |
| Express | 31, 32 | C6526T | H477Y | 21.5 | 0 |
| Express | 31, 32 | G6538A | Splice | | |
| | | | Junction | | |
| Express | 31, 32 | G6761A | G488D | −0.9 | 0.32 |
| Express | 31, 32 | G6761A | G488D | −0.9 | 0.32 |
| Express | 31, 32 | G6793A | A499T | 18.7 | 0 |
| Express | 31, 32 | G6796A | V500I | 5.8 | 0.15 |
| Express | 31, 32 | G6844A | D516N | 1.2 | 0.42 |
| Express | 31, 32 | C6854T | S519F | 11.1 | 0 |
| Express | 31, 32 | G6860A | G521D | 15.5 | 0 |
| Express | 31, 32 | G6860A | G521D | 15.5 | 0 |
| Express | 31, 32 | G6862A | E522K | 20.2 | 0 |
| Express | 31, 32 | G6881A | Intron | | |
| Express | 31, 32 | C6898T | Intron | | |

In one embodiment, the invention relates to a polynucleotide of the SBEIIa gene of the D genome with one or more non-transgenic mutations listed in Table 3 and corresponding to SEQ ID NO: 5. In another embodiment, the polynucleotide with one or more non-transgenic mutations listed in Table 3 is 85%, 86%, 87%, 88%, 89%, 90%, 91%, 92%, 93%, 94%, 95%, 96%, 97%, 98%, 99% or greater than 99% identical to SEQ ID NO: 5. In yet another embodiment, the polynucleotide with one or more non-transgenic mutations listed in Table 3 is 85%, 86%, 87%, 88%, 89%, 90%, 91%, 92%, 93%, 94%, 95%, 96%, 97%, 98%, 99% or greater than 99% similar to SEQ ID NO: 5.

In still another embodiment, the polynucleotide with one or more non-transgenic mutation listed in Table 3 codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical to SEQ ID NO: 6. In still another embodiment, the polynucleotide with one or more non-transgenic mutation listed in Table 3 codes for a SB Ella protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% similar to SEQ ID NO: 6.

### 2. SBEIIb Genes

In another embodiment, one or more non-transgenic mutations are in the SBEIIb gene. In one embodiment, the invention relates to multiple non-transgenic mutations in the SBEIIb gene including but not limited to 1, 2, 3, 4, 5, 6, 7, 8, 9, 10, and greater than 10 mutations.

In still another embodiment, one or more mutations are in the SBEIIb gene of the A genome. In another embodiment, one or more mutations are in the SBEIIb gene of the B genome. In still another embodiment, one or more mutations are in the SBEIIb gene of the D genome. In yet another embodiment, one or more mutations are in the SBEIIb genes of the A and B genomes. In still another embodiment, one or more mutations are in the SBEIIb genes of the A and D genomes. In another embodiment, one or more mutations are in the SBEIIb genes of the B and D genomes. In yet another embodiment, one or more mutations are in the SBEIIb genes of the A, B, and D genomes.

In one embodiment, one or more non-transgenic mutations are in both alleles of the SBEIIb gene in the A genome. In another embodiment, the non-transgenic mutations are identical in both alleles of the SBEIIb gene of the A genome.

In one embodiment, one or more non-transgenic mutations are in both alleles of the SBEIIb gene in the B genome. In another embodiment, the non-transgenic mutations are identical in both alleles of the SBEIIb gene of the B genome.

In one embodiment, one or more non-transgenic mutations are in both alleles of the SBEIIb gene in the D genome. In another embodiment, the non-transgenic mutations are identical in both alleles of the SBEIIb gene of the D genome.

Examples of mutations created and identified in SBEIIb in the A genome of wheat plants are provided in Table 4. Nucleotide and amino acid changes are identified according to their positions in SEQ ID NOs: 7 and 8, respectively.

TABLE 4
Representative Mutations in SBEIIb in the A genome

| | Primer SEQ | Nucleotide | A.A. | | |
|---|---|---|---|---|---|
| Variety | IDs. | Mutation | Mutation | PSSM | SIFT |
| Express | 33, 34 | G211A | Intron | | |
| Express | 33, 34 | G278A | W59* | | |
| Express | 33, 34 | G298A | G66D | 6.1 | 0.03 |
| Express | 33, 34 | G310A | G70E | 2.1 | 0.83 |
| Express | 33, 34 | G310A | G70E | 2.1 | 0.83 |
| Express | 33, 34 | C437T | Intron | | |
| Express | 33, 34 | G485A | Intron | | |
| Express | 33, 34 | G547A | V99I | | 0.84 |
| Express | 33, 34 | G565A | E105K | | 0.11 |
| Express | 33, 34 | G678A | T142= | | |
| Express | 33, 34 | G680A | G143E | | 1 |
| Express | 33, 34 | G709A | G153R | 8.6 | 0.03 |
| Express | 33, 34 | C739T | P163S | 10.2 | 0.09 |
| Express | 33, 34 | C743T | T164M | −3.4 | 0.21 |
| Express | 33, 34 | G769A | E173K | −4.1 | 0.56 |
| Express | 35, 36 | G1237A | E201K | 16.7 | 0.21 |
| Express | 35, 36 | C1307T | Intron | | |
| Express | 35, 36 | C1319T | Intron | | |
| Express | 35, 36 | C1322T | Intron | | |
| Express | 35, 36 | G1341A | G211S | 14.9 | 0.02 |
| Express | 35, 36 | G1356A | E216K | 22.3 | 0 |
| Express | 35, 36 | C1857T | Intron | | |
| Express | 37, 38 | C2021T | Intron | | |
| Express | 37, 38 | C2021T | Intron | | |
| Express | 35, 36 | G2031A | Intron | | |
| Express | 37, 38 | C2072T | Intron | | |
| Express | 37, 38 | C2124T | S259L | | 0.03 |
| Express | 37, 38 | C2126T | P260S | | 0.23 |
| Express | 37, 38 | G2142A | G265D | 3.6 | 0.17 |
| Express | 37, 38 | G2142A | G265D | 3.6 | 0.17 |
| Express | 37, 38 | G2142A | G265D | 3.6 | 0.17 |
| Express | 37, 38 | G2156A | Splice | | |
| | | | Junction | | |
| Express | 37, 38 | C2169T | Intron | | |
| Express | 37, 38 | C2174T | Intron | | |
| Express | 37, 38 | G2244A | G273S | 0.6 | 0.31 |
| Express | 37, 38 | G2245A | G273D | −9.5 | 1 |
| Express | 37, 38 | C2250T | P275S | 11.4 | 0.13 |
| Express | 37, 38 | G2282A | W285* | | |
| Express | 37, 38 | G2282A | W285* | | |
| Express | 37, 38 | G2282A | W285* | | |
| Express | 37, 38 | C2293T | S289F | 8.4 | 0.02 |
| Express | 37, 38 | C2340T | P305S | 15.8 | 0 |
| Express | 37, 38 | C2344T | P306L | 17.3 | 0 |
| Express | 37, 38 | C2344T | P306L | 17.3 | 0 |
| Express | 37, 38 | G2349A | E308K | | 0.07 |
| Express | 37, 38 | A2441T | Intron | | |
| Express | 37, 38 | C2484T | Intron | | |
| Express | 37, 38 | G2525A | Intron | | |
| Express | 37, 38 | G2535A | E309K | | 0.03 |
| Express | 37, 38 | G2540A | K310= | | |
| Express | 37, 38 | C2556T | P316S | 11.5 | 0.07 |
| Express | 37, 38 | C2606T | G332= | | |
| Express | 37, 38 | C2606T | G332= | | |
| Express | 37, 38 | C2617T | P336L | 18.2 | 0.01 |
| Express | 37, 38 | C2642T | Intron | | |
| Express | 37, 38 | G2697A | Intron | | |

In one embodiment, the invention relates to a polynucleotide of the SBEIIb gene of the A genome with one or more non-transgenic mutations listed in Table 4 and corresponding to SEQ ID NO: 7. In another embodiment, the polynucleotide with one or more non-transgenic mutations listed in Table 4 is 85%, 86%, 87%, 88%, 89%, 90%, 91%, 92%, 93%, 94%, 95%, 96%, 97%, 98%, 99% or greater than 99% identical to SEQ ID NO: 7. In yet another embodiment, the polynucleotide with one or more non-transgenic mutations listed in Table 4 is 85%, 86%, 87%, 88%, 89%, 90%, 91%, 92%, 93%, 94%, 95%, 96%, 97%, 98%, 99% or greater than 99% similar to SEQ ID NO: 7.

In still another embodiment, the polynucleotide with one or more non-transgenic mutation listed in Table 4 codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical to SEQ ID NO: 8. In still another embodiment, the polynucleotide with one or more non-transgenic mutation listed in Table 4 codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% similar to SEQ ID NO: 8.

Examples of mutations created and identified in SBEIIb in the B genome of wheat plants are provided in Table 5. Nucleotide and amino acid changes are identified according to their positions in SEQ ID NOs: 9 and 10, respectively.

TABLE 5
Representative mutations in the SBEIIb gene in the B genome

| | Primer SEQ | Nucleotide | A.A. | | |
|---|---|---|---|---|---|
| Variety | IDs. | Mutation | Mutation | PSSM | SIFT |
| Express | 41, 42 | G371A | G58R | | 0.26 |
| Express | 41, 42 | C422T | P75S | 20.4 | 0.02 |
| Express | 41, 42 | G435A | S79N | | 0.31 |
| Express | 41, 42 | C1033T | Intron | | |
| Express | 41, 42 | C1102T | Intron | | |
| Express | 41, 42 | C1102T | Intron | | |
| Express | 41, 42 | G1209A | D129N | | 0.48 |
| Express | 41, 42 | C1246T | S141F | | 0.07 |
| Express | 41, 42 | G1254A | E144K | | 0.91 |
| Express | 43, 44 | G1916A | S208N | | |
| Express | 43, 44 | C2196T | Intron | | |
| Express | 43, 44 | C2206T | Intron | | |
| Express | 43, 44 | G2221A | A225T | 6.9 | 0.21 |
| Express | 45, 46 | C2669T | Intron | | |
| Express | 45, 46 | C2776T | P260S | 10.4 | 0.21 |
| Express | 45, 46 | C2786T | P263L | 25.5 | 0.00 |
| Express | 45, 46 | C2786T | P263L | 25.5 | 0.00 |
| Express | 45, 46 | C2919T | S281L | 9.9 | 0.09 |
| Express | 45, 46 | C2786T | P263L | 25.5 | 0.00 |
| Express | 45, 46 | G3216A | K319= | | |
| Express | 45, 46 | C3232T | R325W | 27.3 | 0.00 |
| Express | 45, 46 | G3260A | S334N | 21.8 | 0.00 |
| Express | 47, 48 | C3478T | Intron | | |
| Express | 47, 48 | G3519A | Intron | | |
| Express | 47, 48 | G3678A | Intron | | |
| Express | 47, 48 | G3814A | Intron | | |
| Express | 47, 48 | C3884T | Intron | | |
| Express | 47, 48 | C3993T | L357F | 8.5 | 0.11 |
| Express | 47, 48 | G4087A | Intron | | |
| Express | 47, 48 | C4419T | Intron | | |
| Express | 47, 48 | G4280A | Intron | | |
| Express | 47, 48 | C4298T | Intron | | |
| Express | 47, 48 | C4374T | Intron | | |
| Express | 47, 48 | C4374T | Intron | | |
| Express | 47, 48 | C4422T | Intron | | |
| Express | 47, 48 | C4489T | Intron | | |

In one embodiment, the invention relates to a polynucleotide of the SBEIIb gene of the B genome with one or more non-transgenic mutations listed in Table 5 and corresponding to SEQ ID NO: 9. In another embodiment, the polynucleotide with one or more non-transgenic mutations listed in Table 5 is 85%, 86%, 87%, 88%, 89%, 90%, 91%, 92%, 93%, 94%, 95%, 96%, 97%, 98%, 99% or greater than 99% identical to SEQ ID NO: 9. In yet another embodiment, the polynucleotide with one or more non-transgenic mutations listed in Table 5 is 85%, 86%, 87%, 88%, 89%, 90%, 91%, 92%, 93%, 94%, 95%, 96%, 97%, 98%, 99% or greater than 99% similar to SEQ ID NO: 9.

In still another embodiment, the polynucleotide with one or more non-transgenic mutation listed in Table 5 codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical to SEQ ID NO: 10. In still another embodiment, the SBEIIb protein with one or more non-transgenic mutations is 85%, 86%, 87%, 88%, 89%, 90%, 91%, 92%, 93%, 94%, 95%, 96%, 97%, 98%, 99% or greater than 99% similar to SEQ ID NO: 10.

Examples of mutations created and identified in SBEIIb in the D genome of wheat plants are provided in Table 6. Nucleotide and amino acid changes are identified according to their positions in SEQ ID NOs: 11 and 12, respectively.

TABLE 6
Representative mutations in SBEIIb in the D genome

| | Primer SEQ | Nucleotide | A.A. | | |
|---|---|---|---|---|---|
| Variety | IDs. | Mutation | Mutation | PSSM | SIFT |
| Express | 49, 50 | G1691A | G58E | | 0.76 |
| Express | 49, 50 | C1742T | P75L | 17 | 0.01 |
| Express | 49, 50 | A1753G | S79G | 8.8 | 0.17 |
| Express | 49, 50 | T1770C | P84= | | |
| Express | 49, 50 | C1784T | P89L | | 0.28 |
| Express | 49, 50 | C1831T | Intron | | |
| Express | 49, 50 | G1840A | Intron | | |
| Express | 49, 50 | C1844T | Intron | | |
| Express | 49, 50 | C1844T | Intron | | |
| Express | 49, 50 | C2438T | Intron | | |
| Express | 49, 50 | C2438T | Intron | | |
| Express | 49, 50 | C2463T | Intron | | |
| Express | 49, 50 | C2479T | P100S | | 0.32 |
| Express | 49, 50 | T2511A | D110E | | 0.98 |
| Express | 49, 50 | C2548T | Q123* | | |
| Express | 49, 50 | G2575A | D132N | | 0.39 |
| Express | 49, 50 | G2649A | Q156= | | |
| Express | 49, 50 | C2672T | T164M | −5.3 | 0.46 |
| Express | 49, 50 | C2676T | L165= | | |
| Express | 51, 52 | C3142T | Intron | | |
| Express | 51, 52 | C3146T | Intron | | |
| Express | 51, 52 | G3159A | Intron | | |
| Express | 51, 52 | G3185A | R180K | | 1 |
| Express | 51, 52 | G3188A | R181K | | 0.81 |
| Express | 51, 52 | G3226A | D194N | 7 | 0.07 |
| Express | 51, 52 | G3226A | D194N | 7 | 0.07 |
| Express | 51, 52 | G3226A | D194N | 7 | 0.07 |
| Express | 51, 52 | G3229A | V195I | 5.1 | 0.13 |
| Express | 51, 52 | C3237T | S197= | | |
| Express | 51, 52 | C3246T | Y200= | | |
| Express | 51, 52 | G3266A | R207H | 8.9 | 0.52 |
| Express | 51, 52 | G3270A | Splice | | |
| | | | Junction | | |
| Express | 51, 52 | C3279T | Intron | | |
| Express | 51, 52 | C3292T | Intron | | |
| Express | 51, 52 | C3303T | Intron | | |
| Express | 51, 52 | C3318T | Intron | | |
| Express | 51, 52 | C3330T | Intron | | |
| Express | 51, 52 | C3332T | Intron | | |
| Express | 51, 52 | G3345A | A209T | 5.3 | 0.49 |
| Express | 51, 52 | G3345A | A209T | 5.3 | 0.49 |
| Express | 51, 52 | C3346T | A209V | 9.8 | 0.25 |
| Express | 51, 52 | C3346T | A209V | 9.8 | 0.25 |
| Express | 51, 52 | C3346T | A209V | 9.8 | 0.25 |
| Express | 51, 52 | G3364A | R215Q | 17.7 | 0.01 |
| Express | 51, 52 | C3410T | Intron | | |
| Express | 51, 52 | C3410T | Intron | | |
| Express | 51, 52 | C3416T | Intron | | |
| Express | 51, 52 | G3571A | A224T | 16.7 | 0.01 |
| Express | 51, 52 | G3599A | W233* | | |
| Express | 51, 52 | G3628A | Splice | | |
| | | | Junction | | |
| Express | 51, 52 | C3662T | Intron | | |
| Express | 51, 52 | C3662T | Intron | | |
| Express | 53, 54 | C4138T | G265= | | |
| Express | 53, 54 | C4060T | Intron | | |
| Express | 53, 54 | G4080A | G246D | | 0 |
| Express | 53, 54 | C4124T | P261S | | 0.07 |
| Express | 53, 54 | C4142T | R267W | 18 | 0 |
| Express | 53, 54 | G4144A | R267= | | |
| Express | 53, 54 | C4159T | Intron | | |
| Express | 53, 54 | C4197A | Intron | | |
| Express | 53, 54 | C4213T | Intron | | |
| Express | 53, 54 | G4229A | Splice | | |
| | | | Junction | | |
| Express | 53, 54 | G4229A | Splice | | |
| | | | Junction | | |
| Express | 53, 54 | C4246T | P275L | 16.1 | 0.05 |
| Express | 53, 54 | C4246T | P275L | 16.1 | 0.05 |
| Express | 53, 54 | G4260A | D280N | 15.8 | 0.07 |
| Express | 53, 54 | C4280T | I286= | | |
| Express | 53, 54 | G4290A | V290M | 13.3 | 0.01 |
| Express | 53, 54 | C4299T | P293S | 8.1 | 0.29 |
| Express | 53, 54 | G4303A | G294E | 4 | 0.25 |
| Express | 53, 54 | C4311T | P297S | 17.3 | 0.07 |
| Express | 53, 54 | G4347A | Splice | | |
| | | | Junction | | |
| Express | 53, 54 | C4361T | Intron | | |
| Express | 53, 54 | G4515A | Intron | | |
| Express | 53, 54 | C4546T | P316S | 9.2 | 0.13 |
| Express | 53, 54 | C4546T | P316S | 9.2 | 0.13 |
| Express | 53, 54 | C4546T | P316S | 9.2 | 0.13 |
| Express | 53, 54 | C4546T | P316S | 9.2 | 0.13 |
| Express | 53, 54 | C4547T | P316L | 18.1 | 0.01 |
| Express | 53, 54 | C4573T | R325W | 22.1 | 0 |
| Express | 53, 54 | C4605T | S335= | | |
| Express | 53, 54 | G4609A | Splice | | |
| | | | Junction | | |
| Express | 53, 54 | G4609A | Splice | | |
| | | | Junction | | |
| Express | 53, 54 | C4618T | Intron | | |
| Express | 57, 58 | C7427T | D425= | | |
| Express | 57, 58 | C7450T | T433M | 12.8 | 0 |
| Express | 57, 58 | G7471A | G440D | 2.1 | 0.26 |
| Express | 57, 58 | C7488T | H446Y | 23.3 | 0 |
| Express | 57, 58 | C7506T | R452C | 25.4 | 0 |
| Express | 57, 58 | C7506T | R452C | 25.4 | 0 |
| Express | 57, 58 | G7537A | Intron | | |
| Express | 57, 58 | C7597T | Intron | | |
| Express | 57, 58 | G7635A | R463= | | |
| Express | 57, 58 | G7655A | R470K | 13.6 | 0.05 |
| Express | 57, 58 | G7669A | E475K | 17.2 | 0 |
| Express | 57, 58 | G7685A | G480D | 26 | 0 |
| Express | 57, 58 | C7689T | F481= | | |
| Express | 57, 58 | G7700A | G485D | 26 | 0 |
| Express | 57, 58 | G7702A | A486T | 5.3 | 0 |
| Express | 57, 58 | C7758T | Intron | | |
| Express | 57, 58 | C7886T | Intron | | |
| Express | 57, 58 | G7897A | V498I | | 0.13 |
| Express | 57, 58 | C7917T | Y504= | | |
| Express | 57, 58 | C7952T | A516V | 18.5 | 0 |
| Express | 57, 58 | G7968A | M521I | 18.9 | 0 |
| Express | 57, 58 | G8056A | Intron | | |

In one embodiment, the invention relates to a polynucleotide of the SBEIIb gene of the D genome with one or more non-transgenic mutations listed in Table 6 and corresponding to SEQ ID NO: 11. In another embodiment, the polynucleotide with one or more non-transgenic mutations listed in Table 6 is 85%, 86%, 87%, 88%, 89%, 90%, 91%, 92%, 93%, 94%, 95%, 96%, 97%, 98%, 99% or greater than 99% identical to SEQ ID NO: 11. In yet another embodiment, the polynucleotide with one or more non-transgenic mutations listed in Table 6 is 85%, 86%, 87%, 88%, 89%, 90%, 91%, 92%, 93%, 94%, 95%, 96%, 97%, 98%, 99% or greater than 99% similar to SEQ ID NO: 11.

In still another embodiment, the polynucleotide with one or more non-transgenic mutation listed in Table 6 codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical to SEQ ID NO: 12. In still another embodiment, the SBEIIb protein with one or more non-transgenic mutations is 85%, 86%, 87%, 88%, 89%, 90%, 91%, 92%, 93%, 94%, 95%, 96%, 97%, 98%, 99% or greater than 99% similar to SEQ ID NO: 12.

### 3. Mutations in Both SBEIIa and SBEIIb Genes

In one embodiment, the invention relates to multiple non-transgenic mutations in the SBEIIa gene including but not limited to 1, 2, 3, 4, 5, 6, 7, 8, 9, 10, and greater than 10 mutations and multiple non-transgenic mutations in the SBEIIb gene including but not limited to 1, 2, 3, 4, 5, 6, 7, 8, 9, 10, and greater than 10 mutations.

In still another embodiment, one or more mutations are in each of the SBEIIa and SBEIIb genes of the A genome. In one embodiment, the invention relates to multiple non-transgenic mutations in the SBEIIa gene including but not limited to 1, 2, 3, 4, 5, 6, 7, 8, 9, 10, and greater than 10 mutations and multiple non-transgenic mutations in the SBEIIb gene including but not limited to 1, 2, 3, 4, 5, 6, 7, 8, 9, 10, and greater than 10 mutations.

In another embodiment, one or more mutations are in each of the SBEIIa and SBEIIb genes of the B genome. In still another embodiment, one or more mutations are in each of the SBEIIa and SBEIIb genes of the D genome. In yet another embodiment, one or more mutations are in each of the SBEIIa and SBEIIb genes of the A and B genomes. In still another embodiment, one or more mutations are in each of the SBEIIa and SBEIIb genes of the A and D genomes. In another embodiment, one or more mutations are in each of the SB Ella and SBEIIb genes of the B and D genomes. In yet another embodiment, one or more mutations are in each of the SBEIIa and SBEIIb genes of the A, B, and D genomes. In yet another embodiment, one or more mutations are in each of the SBEIIa genes of the A, B, and D genomes and additional mutations are in more or more of the SBEIIb genes of the A, B, and D genomes.

### B. SBEII Proteins

Starch is a mixture of amylose and amylopectin, both of which are Glc polymers. Amylose is a mostly linear polymer of 200 to 2000 α-1,4-bonded Glc moieties with rare α-1,6 branch points (for reviews, see Martin and Smith, 1995; Ball et al., 1996). A mylopectin is highly α-1,6-branched, with a complex structure of 10⁶ to 10⁸ Mᵣ and up to 3×10⁶ Glc subunits, making it one of the largest biological molecules in nature.

In the plant, starch is deposited as starch granules in chloroplasts of photosynthetic tissues or in amyloplasts of endosperm, embryos, tubers, and roots. In most plants, starch consists of 20% to 30% amylose and 70% to 80% amylopectin. In photosynthetic and nonphotosynthetic tissues the Glc moiety of ADP-Glc is incorporated in the growing amylose polymer with the help of starch synthases. The formation of α-1,6 linkages in amylopectin is catalyzed by SBEs.

In yet another embodiment, the invention relates to one or more non-trangenic mutations in the SBEII gene (as discussed above in the section entitled SBEII Mutations) that result in an SBEII protein with one or more mutations as compared to wild type SBEII protein. In one embodiment, the non-trangenic mutations include but are not limited to the mutations recited in Tables 1-6 and 8-12, corresponding mutations in homoeologues, and combinations thereof.

In another embodiment, the invention relates to one or more non-transgenic mutations in the SBEII gene that inhibits production of the SBEII protein. In some embodiments, a mutation in the SBEII gene inhibits expression of the SBEII protein. In other embodiments, a mutation in the SBEII gene creates an unstable or reduced function SBEII protein.

In another embodiment, the expression level of SBEII protein with one or more mutations disclosed herein is reduced to 0-2%, 2-5%, 5-7%, 7-10%, 10-15%, 15-20%, 20-25%, 25-30%, 30-35%, 35-40%, 40-45%, 45-50%, 50-60%, 60-70%, 70-80%, 80-90%, 90-95%, and 95-99% of the expression level of the wild type SBEII protein.

In yet another embodiment, the expression level of SB Ella protein with one or more mutations disclosed herein is reduced to 0-2%, 2-5%, 5-7%, 7-10%, 10-15%, 15-20%, 20-25%, 25-30%, 30-35%, 35-40%, 40-45%, 45-50%, 50-60%, 60-70%, 70-80%, 80-90%, 90-95%, and 95-99% of the expression level of the wild type SBEIIa protein.

In still another embodiment, the expression level of SBEIIb protein with one or more mutations disclosed herein is reduced to 0-2%, 2-5%, 5-7%, 7-10%, 10-15%, 15-20%, 20-25%, 25-30%, 30-35%, 35-40%, 40-45%, 45-50%, 50-60%, 60-70%, 70-80%, 80-90%, 90-95%, and 95-99% of the expression level of the wild type SBEIIb protein.

In yet another embodiment, the activity of the SBEII protein with one or more mutations disclosed herein is reduced to 0-1, 1, 2, 3, 4, 5, 6, 7, 8, 9, 10, 11, 12, 13, 14,15, 16, 17,18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 69, 70, 71, 72, 73, 74, 75, 76, 77, 78, 79, 80, 81, 82, 83, 84, 85, 86, 87, 88, 89, 90, 91, 92, 93, 94, 95, 86, 97, 98, 99% and greater than 99% of the activity level of the wild type SBEII protein. In another embodiment, the SBEII protein with one or more mutations disclosed herein has no activity or zero activity as compared to wild type SBEII protein.

In still another embodiment, the activity of the SBEIIa protein with one or more mutations disclosed herein is reduced to 0-1, 2, 3, 4, 5, 6, 7, 8, 9, 10, 11, 12, 13, 14,15, 16, 17,18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 69, 70, 71, 72, 73, 74, 75, 76, 77, 78, 79, 80, 81, 82, 83, 84, 85, 86, 87, 88, 89, 90, 91, 92, 93, 94, 95, 86, 97, 98, 99% and greater than 99% of the activity level of the wild type SBEIIa protein. In another embodiment, the SBEIIa protein with one or more mutations disclosed herein has no activity or zero activity as compared to wild type SBEIIa protein.

In yet another embodiment, the activity of the SBEII b protein with one or more mutations disclosed herein is reduced to 0-1, 2, 3, 4, 5, 6, 7, 8, 9, 10, 11, 12, 13, 14,15, 16, 17,18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 69, 70, 71, 72, 73, 74, 75, 76, 77, 78, 79, 80, 81, 82, 83, 84, 85, 86, 87, 88, 89, 90, 91, 92, 93, 94, 95, 86, 97, 98, 99% and greater than 99% of the activity level of the wild type SBEIIb protein. In another embodiment, the SBEIIb protein with one or more mutations disclosed herein has no activity or zero activity as compared to wild type SBEIIb protein.

### C. Wheat Cultivars

In one embodiment, a wheat cultivar having at least one SBEII gene that is diploid, polyploid, tertraploid, and hexaploid may be used.

In another embodiment, the wheat is Triticum aestivum.

In one embodiment, any cultivar of wheat can be used to create mutations in an SBEII gene. In one embodiment, any cultivar of wheat can be used to create mutations in an SBEIIa gene. In another embodiment, any cultivar of wheat can be used to create mutations in an SBEIIb gene.

In one embodiment, any cultivar of wheat can be used as lines to cross SBEII mutations into different cultivars. In still another embodiment, any cultivar of wheat can be used as lines to cross SBEIIa mutations into different cultivars. In another embodiment, any cultivar of wheat can be used as lines to cross SBEIIb mutations into different cultivars.

In another embodiment, any cultivar of wheat having at least one SBEII gene may be used including but not limited to hard red spring wheat, hard white wheat, durum wheat, soft white spring wheat, soft white winter wheat, hard red winter wheat, common wheat, splelt wheat, emmer wheat, pasta wheat and turgidum wheat.

In one embodiment, hard red spring wheat includes but is not limited to Bullseye, Cabernet, Cal Rojo, Hank, Joaquin, Kelse, Lariat, Lassik, Malbec, Mika, PR 1404, Redwing, Summit 515, SY 314, Triple IV, Ultra, WB-Patron, WB-Rockland, Yecora Rojo, Accord, Aim, Anza, Baker, Beth Hashita, Bonus, Borah, Brim, Brooks, Buck Pronto, Butte 86, Cavalier, Challenger, Chief, Ciano T79, Colusa, Companion, Copper, Cuyama, Dash 12, Eldon, Enano, Express, Expresso, Jefferson, Genero F81, Grandin, Helena 554, Hollis, Imuris T79, Inia 66R, Jerome, Kern, Len, Marshall, McKay, Nomad, Northwest 10, Oslo, Pavon F76, Pegasus, Pitic 62, Poco Red, Powell, Probrand 711, Probrand 751, Probrand 771, Probrand 775, Probred, Prointa Queguay, Prointa Quintal, Rich, RSI 5, Sagittario, Scarlet, Serra, Shasta, Solano, Spillman, Sprite, Stander, Stellar, Stoa, Success, Summit, Sunstar 2, Sunstar King, Tadinia, Tammy, Tanori 71, Tara 2000, Tempo, Tesia T79, Topic, UI Winchester, Vance, Vandal, W444, Wampum, Wared, WB-Fuzion, Westbred 906R, Westbred 911, Westbred 926, Westbred 936, Westbred Discovery, Westbred Rambo, Y olo, and Zeke.

In another embodiment, hard white wheat includes but is not limited to Blanca Fuerte, Blanca Grande 515, Blanca Royale, ClearWhite, Patwin, Patwin 515, WB-Cristallo, WB-Paloma, WB-Perla, Alta Blanca, Blanca Grande, Delano, Golden Spike, ID377S, Klasic, Lochsa, Lolo, Macon, Otis, Phoenix, Pima 77, Plata, Pristine, Ramona 50, Siete Cerros 66, Vaiolet, and Winsome.

In yet another embodiment, durum wheat includes but is not limited to Crown, Desert King, Desert King HP, Duraking, Fortissimo, Havasu, Kronos, Maestrale, Normanno, Orita, Platinum, Q-M ax, RSI 59, Saragolla, Tango, Tipai, Topper, Utopia, Volante, WB-Mead, Westmore, Aldente, Aldura, Altar 84, Aruba, Bittern, Bravadur, Candura, Cortez, Deluxe, Desert Titan, Durex, Durfort, Eddie, Germains 5003D, Imperial, Kofa, Levante, Matt, Mead, Mexicali 75, Minos, Modoc, Mohawk, Nudura, Ocotillo, Produra, Reva, Ria, Septre, Sky, Tacna, Titan, Trump, Ward, Westbred 803, Westbred 881, Westbred 883, Westbred 1000D, Westbred Laker, Westbred Turbo, and Y avaros 79.

In another embodiment, soft white spring wheat includes but is not limited to A Ipowa, Alturas, Babe, Diva, J D, New Dirkwin, Nick, Twin, Whit, Blanca, Bliss, Calorwa, Centennial, Challis, Dirkwin, Eden, Edwall, Fielder, Fieldwin, jubilee, Louise, Owens, Penawawa, Pomerelle, Sterling, Sunstar Promise, Super Dirkwin, Treasure, UI Cataldo, UI Pettit, Urquie, V anna, Waduel, Waduel 94, Wakanz, Walladay, Wawawai, Whitebird, and Zak.

In still another embodiment, soft white winter wheat includes but is not limited to AP Badger, AP Legacy, Brundage 96, Bruneau, Cara, Goetze, Legion, Mary, Skiles, Stephens, SY Ovation, Tubbs, WB-J unction, WB-528, Xerpha, Yamhill, Barbee, Basin, Bitterroot, Bruehl, Castan, Chukar, Coda, Daws, Edwin, Eltan, Faro, Finch, Foote, Gene, Hill 81, Hiller, Hubbard, Hyak, Hyslop, Idaho 587, Kmor, Lambert, Lewjain, MacVicar, Madsen, Malcolm, Masami, McDermid, Moro, Nugaines, ORCF-101, ORCF-102, ORCF-103, Rod, Rohde, Rulo, Simon, Salute, Temple, Tres, Tubbs 06, UICF-Brundage, WB-523, and Weatherford.

In another embodiment, hard red winter wheat includes but is not limited to Andrews, Archer, Batum, Blizzard, Bonneville, Boundary, Declo, Deloris, Finley, Garland, Hatton, Hoff, Longhorn, Manning, Meridian, Promontory, Vona, Wanser, Winridge.

In another embodiment, common wheat (hexaploid, free threshing), Triticum aestivum ssp aestivum includes but is not limited to Sonora, Wit Wolkoring, Chiddam Blanc De Mars, India-Jammu, Foisy.

In still another embodiment, spelt wheat (hexaploid, not free threshing), Triticum aestivum ssp spelta includes but is not limited to Spanish Spelt, Swiss Spelt.

In yet another embodiment, Emmer Wheat (tetraploid), Triticum turgidum ssp. dicoccum includes but is not limited to Ethiopian Blue Tinge.

In another embodiment, pasta wheat (tetraploid, free threshing), Triticum turgidum ssp durum includes but is not limited to Blue Beard, Durum-Iraq.

In yet another embodiment, Turgidum Wheat (tetraploid, free threshing), Triticum turgidum ssp turgidum includes but is not limited to Akmolinka, Maparcha.

In one embodiment, a cultivar of wheat having at least one SBEII gene with substantial percent identity to SEQ ID NO: 1, SEQ ID NO: 3, SEQ ID NO: 5, SEQ ID NO: 7, SEQ ID NO: 9, or SEQ ID NO: 11 may be used in the invention.

As used herein with regard to the wheat cultivars, “substantial percent identity” means that the DNA sequence of the gene is sufficiently similar to SEQ ID NO: 1, 3, 5, 7, 9, or 11 at the nucleotide level to code for a substantially similar protein, allowing for allelic differences (or alternate mRNA splicing) between cultivars. In accordance with one embodiment of the invention, “substantial percent identity” may be present when the percent identity in the coding region between the SBEII gene and SEQ ID NO: 1, 3, 5, 7, 9, or 11 is as low as about 85%, provided that the percent identity in the conserved regions of the gene is higher (e.g., at least about 90%). Preferably the percent identity in the coding region is 85-90%, more preferably 90-95%, and optimally, it is above 95%. Thus, one of skill in the art may prefer to utilize a wheat cultivar having commercial popularity or one having specific desired characteristics in which to create the SBEII-mutated wheat plants, without deviating from the scope and intent of the present invention. Alternatively, one of skill in the art may prefer to utilize a wheat cultivar having few polymorphisms, such as an in-bred cultivar, in order to facilitate screening for mutations within one or more SBEII genes in accordance with the present invention.

### Representative Methodology for Identification of SBEII Mutations

In order to create and identify the SBEII mutations and wheat plants of the invention, a method known as TILLING was utilized. See McCallum et al., Nature Biotechnology 18:455-457, 2000; McCallum et al., Plant Physiology, 123:439-442, 2000; U.S. Publication No. 20040053236; and U.S. Pat. No. 5,994,075, all of which are incorporated herein by reference. In the basic TILLING methodology, plant materials, such as seeds, are subjected to chemical mutagenesis, which creates a series of mutations within the genomes of the seeds' cells. The mutagenized seeds are grown into adult M1 plants and self-pollinated. DNA samples from the resulting M2 plants are pooled and are then screened for mutations in a gene of interest. Once a mutation is identified in a gene of interest, the seeds of the M2 plant carrying that mutation are grown into adult M3 plants and screened for the phenotypic characteristics associated with the gene of interest.

The hexaploid cultivar Express and the tetraploid cultivar Kronos were used.

In one embodiment, seeds from wheat are mutagenized and then grown into M1 plants. The M1 plants are then allowed to self-pollinate and seeds from the M1 plant are grown into M2 plants, which are then screened for mutations in their SBEII loci. While M1 plants can be screened for mutations in accordance with alternative embodiments of the invention, one advantage of screening the M2 plants is that all somatic mutations correspond to germline mutations.

One of skill in the art will understand that a variety of wheat plant materials, including, but not limited to, seeds, pollen, plant tissue or plant cells, may be mutagenized in order to create the SBEII-mutated wheat plants of the invention. However, the type of plant material mutagenized may affect when the plant DNA is screened for mutations. For example, when pollen is subjected to mutagenesis prior to pollination of a non-mutagenized plant, the seeds resulting from that pollination are grown into M1 plants. Every cell of the M1 plants will contain mutations created in the pollen, thus these M1 plants may then be screened for SBEII mutations instead of waiting until the M2 generation.

Mutagens that create primarily point mutations and short deletions (about 1 to about 30 nucleotides), insertions, transversions, and or transitions, such as chemical mutagens or radiation, may be used to create the mutations. Mutagens conforming with the method of the invention include, but are not limited to, ethyl methanesulfonate (EMS), methylmethane sulfonate (MMS), N-ethyl-N-nitrosourea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitrosamine, N-methyl-N′-nitro-N itrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9[3-(ethyl-2-chloro-ethyl)aminopropylamino]acridine dihydrochloride (ICR-170), and formaldehyde. Spontaneous mutations in an SBEII gene that may not have been directly caused by the mutagen can also be identified.

Any suitable method of plant DNA preparation now known or hereafter devised may be used to prepare the wheat plant DNA for SBEIIa and SBEIIb mutation screening. For example, see Chen & Ronald, Plant Molecular Biology Reporter 17:53-57, 1999; Stewart and Via, Bio Techniques 14:748-749, 1993. Additionally, several commercial kits designed for this purpose are available, including kits from Qiagen (Valencia, CA) and Qbiogene (Carlsbad, CA).

In one embodiment, prepared DNA from individual wheat plants are pooled in order to expedite screening for mutations in one or more SBEII genes of the entire population of plants originating from the mutagenized plant tissue. The size of the pooled group may be dependent upon the sensitivity of the screening method used. Preferably, groups of two or more individual wheat plants are pooled.

In another embodiment, after the DNA samples are pooled, the pools are subjected to SBEIIa or SBEIIb sequence-specific amplification techniques, such as Polymerase Chain Reaction (PCR). For a general overview of PCR, see PCR Protocols: A Guide to Methods and Applications (Innis, Gelfand, Sninsky, and White, eds.), Academic Press, San Diego, 1990.

Any primer specific to an SBEIIa locus or an SBEIIb locus or the sequences immediately adjacent to one of these loci may be utilized to amplify the SBEII sequences within the pooled DNA sample. Preferably, the primer is designed to amplify the regions of the SBEII locus where useful mutations are most likely to arise. Most preferably, the primer is designed to detect exonic regions of one or more SBEII genes. Additionally, it is preferable for the primer to target known polymorphic sites to design genome specific primers in order to ease screening for point mutations in a particular genome. To facilitate detection of PCR products on a gel, the PCR primer may be labeled using any conventional or hereafter devised labeling method.

In one embodiment, primers are designed based upon the SBEIIa and SBEIIb homoeologs (SEQ ID NOs: 1, 3,5, 7,9, and 11). Exemplary primers (SEQ ID NOs: 13-58) that have proven useful in identifying useful mutations within the SBEIIa and SBEIIb sequences are shown below in Table 1. These primers are also detailed in the Sequence Listing appended hereto.

TABLE 7
Exemplary Primers

| SEQ | | |
|---|---|---|
| ID | | |
| NO | Region Screened | Sequence |
| 13 | Sbe2a_A_Exon2-3 | ACGGCTTTGATCATCTCCTCCCA |
| 14 | Sbe2a_A_Exon2-3 | TTTGTCTCTTTGATGTTCCCCAAAT |
| 15 | Sbe2a_A_Exon7-9 | TATGACCAGAGTATGTCTACAGCTTGGCAAT |
| 16 | Sbe2a_A_Exon7-9 | TGCATCCTAAGTGGGAAACCCTAACCA |
| 17 | Sbe2a_A_Exon12-14 | TCAATTTGGATCAGAGGGGATAGTCCA |
| 18 | Sbe2a_A_Exon12-14 | TGACAAGGTTGCCCATTTCTAATGCAA |
| 19 | Sbe2a_B_Exon2-3 | GATAGCTGGATTAGGCGATCGCCTCAGG |
| 20 | Sbe2a_B_Exon2-3 | TTGGTAGAGGAATTAGCAAAGTAAAATCCA |
| 21 | Sbe2a_B_Exon7-9 | GGTAGAACCTTTTGCATTATGTGTGCTTTTCC |
| 22 | Sbe2a_B_Exon7-9 | GCTACCTCGAAATGCAATGGAAATCTTAGAGAC |
| 23 | Sbe2a_B_Exon12-14 | CCAAGGAGGGAGTGAGGAGCTTGACTT |
| 24 | Sbe2a_B_Exon12-14 | TGTCAGCTTGAATGCCCTTGCACTTCT |
| 25 | Sbe2a_D_Exon2-3 | GATCGCGCTTCCTGAACCTGTAT |
| 26 | Sbe2a_D_Exon2-3 | CTCAGACCACGAAGGGATCTGTATG |
| 27 | Sbe2a_D_Exon7-9 | ATGAATACGTGCAACACTCCCATCTGC |
| 28 | Sbe2a_D_Exon7-9 | GGAAGCAAAGTTTTGCACTTGCCAATATG |
| 29 | Sbe2a_D_Exon10-11 | CGTCTCCAGCAAGCCATTTCCTACCTTA |
| 30 | Sbe2a_D_Exon10-11 | TTTTGCCACTAGTTTTTGCCAATTTTCC |
| 31 | Sbe2a_D_Exon12-14 | TCAATCAATTTGGATCAGAGGGAACATCA |
| 32 | Sbe2a_D_Exon12-14 | TAGCAGTGCAGGAATTTAAGTTAAACCACTATTACA |
| 33 | Sbe2b_A_Exon2-3 | CTCCCATTCTCGTTTATTCGTAGC |
| 34 | Sbe2b_A_Exon2-3 | GTTCGGTTACCATGTCACCTCAGAGC |
| 35 | Sbe2b_A_Exon4-7 | GCCAATTGAACAACAATGCCACTTCATT |
| 36 | Sbe2b_A_Exon4-7 | GAGTACCCATTCGCACCTAGATGT |
| 37 | Sbe2b_A_Exon7-9 | GCCTGTTGCACGAGCCCATTAATTACT |
| 38 | Sbe2b_A_Exon7-9 | TTCGAACAAATGGACACCAGCTTTTGAT |
| 39 | Sbe2b_A_Exon10-11 | TTATATATCAACTTATGAATCCTGAACG |
| 40 | Sbe2b_A_Exon10-11 | GTAAAGTGTTCTTTTAGCAATTTATACAAAC |
| 41 | Sbe2b_B_Exon1-3 | GCCTCCTCATTTCGCTCGCGTGGGTTTAAG |
| 42 | Sbe2b_B_Exon1-3 | AGTGACTATGAACTTCAAGAATTTCGTGATACATCA |
| 43 | Sbe2b_B_Exon4-6 | CTACAAAAAATTGAACAACGATGCCACTTCAT |
| 44 | Sbe2b_B_Exon4-6 | CCAACTATATTTACAGCTCAACTCTGG |
| 45 | Sbe2b_B_Exon7-9 | ACTGATTTTGTTCTTGCAAGACATTCA |
| 46 | Sbe2b_B_Exon7-9 | CAAATGGACACCAGCTTTTGATGC |
| 47 | Sbe2b_B_Exon10-11 | AAAGTTAGCTATATGCAGTTTAAGTTAATTTACAGGT |
| 48 | Sbe2b_B_Exon10-11 | TGTAAGATGTTCTTTCAGCAATTTATACTA |
| 49 | Sbe2b_D_Exon2-3 | ACGACGCGTGCCGATTCCGTAT |
| 50 | Sbe2b_D_Exon2-3 | GCCATTCACATCTTATCAAAGACTGTAAATTGTTT |
| 51 | Sbe2b_D_Exon4-7 | ATCCTACAAAAAATTGAACAACAATGCCACTTTC |
| 52 | Sbe2b_D_Exon4-7 | ACATGGAGCTACAGTTCAGATGTGC |
| 53 | Sbe2b_D_Exon7-9 | GCCTGTTGCACGAGCCCATTACTAGAT |
| 54 | Sbe2b_D_Exon7-9 | GGCAATTACTTGTTTCTTTGTGCAATTACTTGTT |
| 55 | Sbe2b_D_Exon10-11 | GTTTTGAATGCTCAAGAGAAGTACTAGT |
| 56 | Sbe2b_D_Exon10-11 | TGTAAGATGTTCTTTCAGCAATTTATACTA |
| 57 | Sbe2b_D_Exon12-14 | TTATGTCTTGGTCCAAAGCCCCTTTTTG |
| 58 | Sbe2b_D_Exon12-14 | TCCACGTCAGGAACTTAGACATGCAACTAT |

In another embodiment, the PCR amplification products may be screened for SBEII mutations using any method that identifies nucleotide differences between wild type and mutant sequences. These may include, for example, without limitation, sequencing, denaturing high pressure liquid chromatography (dHPLC), constant denaturant capillary electrophoresis (CDCE), temperature gradient capillary electrophoresis (TGCE) (see Li et al., Electrophoresis 23(10):1499-1511, 2002), or by fragmentation using enzymatic cleavage, such as used in the high throughput method described by Colbert et al., Plant Physiology 126:480-484, 2001. Preferably, the PCR amplification products are incubated with an endonuclease that preferentially cleaves mismatches in heteroduplexes between wild type and mutant sequences.

In another embodiment, cleavage products are electrophoresed using an automated sequencing gel apparatus, and gel images are analyzed with the aid of a standard commercial image-processing program.

In yet another embodiment, once an M2 plant having a mutated SBEII gene sequence is identified, the mutations are analyzed to determine their effect on the expression, translation, and/or activity of an SBEII enzyme. In one embodiment, the PCR fragment containing the mutation is sequenced, using standard sequencing techniques, in order to determine the exact location of the mutation in relation to the overall SBEII sequence. Each mutation is evaluated in order to predict its impact on protein function (i.e., from completely tolerated to causing loss-of-function) using bioinformatics tools such as SIFT (Sorting Intolerant from Tolerant; N g and Henikoff, Nucleic Acids Research 31:3812-3814, 2003), PSSM (Position-Specific Scoring Matrix; Henikoff and Henikoff, Computer Applications in the Biosciences 12:135-143, 1996) and PARSESNP (Taylor and Greene, Nucleic Acids Research 31:3808-3811, 2003). For example, a SIFT score that is less than 0.05 and a large change in PSSM score (e.g., roughly 10 or above) indicate a mutation that is likely to have a deleterious effect on protein function. These programs are known to be predictive, and it is understood by those skilled in the art that the predicted outcomes are not always accurate.

In another embodiment, if the initial assessment of a mutation in the M2 plant indicates it to be of a useful nature and in a useful position within an SBEII gene, then further phenotypic analysis of the wheat plant containing that mutation may be pursued. In hexaploid wheat, mutations in each of the A, B and D genomes usually must be combined before a phenotype can be detected. In tetraploid wheat, A and B genome mutations are combined. In addition, the mutation containing plant can be backcrossed or outcrossed two times or more in order to eliminate background mutations at any generation. Then the backcrossed or outcrossed plant can be self-pollinated or crossed in order to create plants that are homozygous for the SBEII mutations.

Several physical characteristics of these homozygous SBEII mutant plants are assessed to determine if the mutation results in a useful phenotypic change in the wheat plant without resulting in undesirable negative effects, such as significantly reduced seed yields.

### Methods of Producing a Wheat Plant

In another embodiment, the invention relates to a method for producing a wheat plant with increased resistant starch levels. In another embodiment, the invention relates to a method for producing a wheat plant with an increased proportion of amylose in the starch.

In another embodiment, the invention relates to a method of out-crossing SBEII gene mutations to wild type wheat. In another embodiment, the invention relates to a method of out-crossing SB Ella gene mutations to wild type wheat. In another embodiment, the invention relates to a method of out-crossing SBEIIb gene mutations to wild type wheat.

In another embodiment, the invention relates to a method for producing a wheat plant having increased amylose content. In still another embodiment, the invention relates to a method for producing a wheat plant having reduced activity of one or more SBEII enzymes compared to the wild type wheat plants.

In one embodiment, the method comprises inducing at least one non-transgenic mutation in at least one copy of an SBEII gene in plant material or plant parts from a parent wheat plant; growing or using the mutagenized plant material to produce progeny wheat plants; analyzing mutagenized plant material and/or progeny wheat plants to detect at least one mutation in at least one copy of a SBEII gene; and selecting progeny wheat plants that have at least one mutation in at least one copy of an SBEII gene.

In another embodiment, the method further comprises crossing progeny wheat plants that have at least one mutation in at least one copy of an SBEII gene with other progeny wheat plants that have at least one mutation in a different copy of an SBEII gene. The process of identifying progeny wheat plants with mutations and crossing said progeny wheat plants with other progeny wheat plants, which have mutations, can be repeated to produce progeny wheat plants with reduced SBEII enzyme activity.

In another embodiment, the level of activity of the SBEII protein in the wheat plant is reduced and selected from the group consisting of 0-2%, 2-5%, 5-7%, 7-10%, 10-15%, 15-20%, 20-25%, 25-30%, 30-35%, 35-40%, 40-45%, 45-50%, 50-60%, 60-70%, 70-80%, 80-90%, 90-95%, 95-99% of the level of activity of the SBEII protein in the wild type plant.

In still another embodiment, the level of activity of the SBEIIa protein in the wheat plant is reduced compared to the wild type plant and is selected from the group consisting of 0-2%, 2-5%, 5-7%, 7-10%, 10-15%, 15-20%, 20-25%, 25-30%, 30-35%, 35-40%, 40-45%, 45-50%, 50-60%, 60-70%, 70-80%, 80-90%, 90-95%, 95-99% of the level of activity of the SBEIIa protein in the wild type plant.

In yet another embodiment, the level of activity of the SBEIIb protein in the wheat plant is reduced and selected from the group consisting of 0-2% 2-5%, 5-7%, 7-10%, 10-15%, 15-20%, 20-25%, 25-30%, 30-35%, 35-40%, 40-45%, 45-50%, 50-60%, 60-70%, 70-80%, 80-90%, 90-95%, 95-99% of the level of activity of the SBEIIb protein in the wild type plant.

A. Methods of Producing a Wheat Plant with One or More Mutations in the SBEIIa Gene in More than One Genome

In still another embodiment, the invention relates to a method for producing a wheat plant comprising inducing at least one non-transgenic mutation in at least one copy of an SBEIIa gene in plant material from a parent wheat plant that comprises a mutation in an SBEIIa gene; growing or using the mutagenized plant material to produce progeny wheat plants; and selecting progeny wheat plants that have at least one mutation in at least two copies of an SBEIIa gene.

For example, the parent wheat plant may have a mutation in an SB Ella gene of the A genome. The selected progeny wheat plants may have a mutation in an SBEIIa gene of the A genome and one or more mutations in the SBEIIa gene of the B genome. This example is provided merely for clarification and should not limit the methods disclosed herein.

In yet another embodiment, the invention relates to a method for producing a wheat plant comprising inducing at least one non-transgenic mutation in at least one copy of an SBEIIa gene in plant material from a parent wheat plant that comprises at least one mutation in two SBEIIa genes; growing or using the mutagenized plant material to produce progeny wheat plants; and selecting progeny wheat plants that have at least one mutation in three copies of an SBEIIa gene. In this embodiment, there would be at least one mutation in the SBEIIa gene of the A, B and D genomes.

In another embodiment, the invention relates to a method for producing a wheat plant comprising crossing a first wheat plant that has at least one non-transgenic mutation in a first SBEIIa gene with a second wheat plant that has at least one non-transgenic mutation in a second SBEIIa gene; and selecting progeny wheat plants that have at least one mutation in at least two copies of an SBEIIa gene.

In another embodiment, the invention relates to a method for producing a wheat plant comprising crossing a first wheat plant that has at least one non-transgenic mutation in a first and second SBEIIa gene with a second wheat plant that has at least one non-transgenic mutation in a third SBEIIa gene; and selecting progeny wheat plants that have at least one mutation in all three copies of an SBEIIa gene. In this embodiment, there would be at least one mutation in the SBEIIa gene of the A, B and D genomes.

In another embodiment, the grain of the wheat plant produced according to the methods disclosed herein comprises starch, and the proportion of amylose in the starch is selected from the group consisting of at least 30%, 30-35%, 35-40%, 40-45%, 45-50%, 50-55%, 55-60%, and 60-65% (w/w). In one embodiment, the proportion of amylose in the starch is 47-60% (w/w).

B. Methods of Producing a Wheat Plant with Mutations in the SBEIIb Gene in More than One Genome

In still another embodiment, the invention relates to a method for producing a wheat plant comprising inducing at least one non-transgenic mutation in at least one copy of an SBEIIb gene in plant material from a parent wheat plant that comprises a mutation in an SBEIIb gene; growing or using the mutagenized plant material to produce progeny wheat plants; and selecting progeny wheat plants that have at least one mutation in at least two copies of an SBEIIb gene.

For example, the parent wheat plant may have a mutation in an SBEIIb gene of the A genome. The selected progeny wheat plants may have a mutation in an SBEIIb gene of the A genome and one or more mutations in the SBEIIb gene of the B genome. This example is provided merely for clarification and should not limit the methods disclosed herein.

In yet another embodiment, the invention relates to a method for producing a wheat plant comprising inducing at least one non-transgenic mutation in at least one copy of an SBEIIb gene in plant material from a parent wheat plant that comprises at least one mutation in two SBEIIb genes; growing or using the mutagenized plant material to produce progeny wheat plants; and selecting progeny wheat plants that have at least one mutation in three copies of an SBEIIb gene. In this embodiment, there would be at least one mutation in the SBEIIb gene of the A, B and D genomes.

In another embodiment, the invention relates to a method for producing a wheat plant comprising crossing a first wheat plant that has at least one non-transgenic mutation in a first SBEIIb gene with a second wheat plant that has at least one non-transgenic mutation in a second SBEIIb gene; and selecting progeny wheat plants that have at least one mutation in at least two copies of an SBEIIb gene.

In another embodiment, the invention relates to a method for producing a wheat plant comprising crossing a first wheat plant that has at least one non-transgenic mutation in a first and second SBEIIb gene with a second wheat plant that has at least one non-transgenic mutation in a third SBEIIb gene; and selecting progeny wheat plants that have at least one mutation in all three copies of an SBEIIb gene. In this embodiment, there would be at least one mutation in the SBEIIb gene of the A, B and D genomes.

In another embodiment, the grain of the wheat plant produced according to the methods disclosed herein comprises starch, and the proportion of amylose in the starch is selected from the group consisting of at least 30%, 30-35%, 35-40%, 40-45%, 45-50%, 55-60%, 60-65%, 65-70%, 70-75%, 75-80%, 80-85%, 85-90%, 90-95%, and greater than 95% (w/w).

C. Methods of Producing a Wheat Plant with One or More Mutations in the SBEIIa Gene and SBEII b Gene in More than One Genome

In one embodiment, the invention relates to a method of producing a wheat plant with one or more mutations in the SBEIIa gene and one or more mutations in the SBEIIb gene in one or more than one genome.

In one embodiment, the wheat plant may comprise one mutation in the SBEIIa gene and 1, 2, 3, 4, 5, 6, 7, 8, 9, 10, or more than 10 mutations in the SBEIIb gene. In one embodiment, the w heat plant may comprise 2 mutations in the SBEIIa gene and 1, 2, 3, 4, 5, 6, 7, 8, 9, 10, or more than 10 mutations in the SBEIIb gene.

In one embodiment, the wheat plant may comprise 3 mutations in the SBEIIa gene and 1, 2, 3, 4, 5, 6, 7, 8, 9, 10, or more than 10 mutations in the SBEIIb gene. In one embodiment, the wheat plant may comprise 4 mutations in the SBEIIa gene and 1, 2, 3, 4, 5, 6, 7, 8, 9, 10, or more than 10 mutations in the SBEIIb gene. In one embodiment, the wheat plant may comprise 5 mutations in the SBEIIa gene and 1, 2, 3, 4, 5, 6, 7, 8, 9, 10, or more than 10 mutations in the SBEIIb gene. In one embodiment, the wheat plant may comprise 6 mutations in the SB Ella gene and 1, 2, 3, 4, 5, 6, 7, 8, 9, 10, or more than 10 mutations in the SBEIIb gene.

In one embodiment, the wheat plant may comprise 7 mutations in the SBEIIa gene and 1, 2, 3, 4, 5, 6, 7, 8, 9, 10, or more than 10 mutations in the SBEIIb gene. In one embodiment, the wheat plant may comprise 8 mutations in the SBEIIa gene and 1, 2, 3, 4, 5, 6, 7, 8, 9, 10, or more than 10 mutations in the SBEIIb gene. In one embodiment, the wheat plant may comprise 9 mutations in the SBEIIa gene and 1, 2, 3, 4, 5, 6, 7, 8, 9, 10, or more than 10 mutations in the SBEIIb gene. In one embodiment, the wheat plant may comprise 10 mutations in the SBEIIa gene and 1, 2, 3, 4, 5, 6, 7, 8, 9, 10, or more than 10 mutations in the SBEIIb gene.

In one embodiment, the invention relates to a method for producing a wheat plant comprising inducing at least one non-transgenic mutation in at least one copy of an SBEIIa and SBEIIb gene in plant material from a parent wheat plant that comprises a mutation in an SBEIIa and SBEIIb genes; growing or using the mutagenized plant material to produce progeny wheat plants; and selecting progeny wheat plants that have at least one mutation in at least two SBEIIa genes and at least one mutation in at least two SBEIIb genes.

For example, the parent wheat plant may have a mutation in SB Ella and SBEIIb genes of the A genome. The selected progeny wheat plants may have a mutation in an SBEIIa and SBEIIb gene of the A genome and one or more mutations in the SBEIIa and SBEIIb genes of the B genome. This example is provided merely for clarification and should not limit the methods disclosed herein.

In yet another embodiment, the invention relates to a method for producing a wheat plant comprising inducing at least one non-transgenic mutation in at least one copy of SBEIIa and SBEIIb genes in plant material from a parent wheat plant that comprises at least one mutation in two SBEIIa genes and at least one mutation in two SBEIIb genes; growing or using the mutagenized plant material to produce progeny wheat plants; and selecting progeny wheat plants that have at least one mutation in three copies of an SBEIIa gene and at least one mutation in three copies of an SBEIIb gene. In this embodiment, there would be at least one mutation in the SBEIIa gene of the A, B and D genomes and at least one mutation in the SBEIIb gene of the A, B and D genomes.

In another embodiment, the invention relates to a method for producing a wheat plant comprising crossing a first wheat plant that has at least one non-transgenic mutation in a first SBEIIa gene and a first SBEIIb gene with a second wheat plant that has at least one non-transgenic mutation in a second SBEIIa gene and a second SBEIIb gene; and selecting progeny wheat plants that have at least one mutation in at least two copies of an SBEIIa and SBEIIb gene.

In another embodiment, the invention relates to a method for producing a wheat plant comprising crossing a first wheat plant that has at least one non-transgenic mutation in a first and second SBEIIa gene and at least one non-transgenic mutation in a first and second SBEIIb gene with a second wheat plant that has at least one non-transgenic mutation in a third SBEIIa and at least one non-transgenic mutation in a third SBEIIb gene; and selecting progeny wheat plants that have at least one mutation in all three copies of an SBEIIa and SBEIIb gene. In this embodiment, there would be at least one mutation in the SBEIIb gene of the A, B and D genomes.

In another embodiment, the grain of the wheat plant produced according to the methods disclosed herein comprises starch, and the proportion of amylose in the starch is selected from the group consisting of at least 30%, 30-35%, 35-40%, 40-45%, 45-50%, and 50-55% (w/w).

### Wheat Plant, Wheat Seed and Parts of Wheat Plant

In one embodiment, a wheat plant is produced according to the methods disclosed herein. In another embodiment, the wheat plant, wheat seed or parts of a wheat plant have one or more mutations in an SBEII gene. In another embodiment, the wheat plant, wheat seed or parts of a wheat plant have one or more mutations in SBEII genes.

In another embodiment, the invention relates to a wheat plant, wheat seed or parts of a wheat plant comprising one or more non-transgenic mutations in the SBEIIa gene. In another embodiment, the invention relates to a wheat plant, wheat seed or parts of a wheat plant comprising at least one non-transgenic mutation in the SB Ella gene in each of two genomes. In still another embodiment, the invention relates to a wheat plant, wheat seed or parts of a wheat plant comprising at least one non-transgenic mutation in the SBEIIa gene in each of three genomes.

In one embodiment, the wheat plant, wheat seed or parts of a wheat plant comprises one or more non-transgenic mutations in both alleles of the SBEIIa gene in the A genome. In another embodiment, the non-transgenic mutations are identical in both alleles of the SBEIIa gene of the A genome.

In one embodiment, the wheat plant, wheat seed or parts of a wheat plant comprises one or more non-transgenic mutations in both alleles of the SB Ella gene in the B genome. In another embodiment, the non-transgenic mutations are identical in both alleles of the SBEIIa gene of the B genome.

In one embodiment, the wheat plant, wheat seed or parts of a wheat plant comprises one or more non-transgenic mutations in both alleles of the SB Ella gene in the D genome. In another embodiment, the non-transgenic mutations are identical in both alleles of the SBEIIa gene of the D genome.

In one embodiment, the invention relates to a wheat plant, wheat seed or parts of a wheat plant comprising a polynucleotide of the SB Ella gene in the A genome with one or more non-transgenic mutations listed in Table 1 and corresponding to SEQ ID NO: 1. In another embodiment, the wheat plant, wheat seed or parts of the wheat plant comprise a polynucleotide with one or more non-transgenic mutations listed in Table 1 and is 85%, 86%, 87%, 88%, 89%, 90%, 91%, 92%, 93%, 94%, 95%, 96%, 97%, 98%, 99% or greater than 99% identical or similar to SEQ ID NO: 1.

In still another embodiment, the wheat plant, wheat seed or parts of a wheat plant comprise a polynucleotide with one or more non-transgenic mutations listed in Table 1 that codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 2.

In one embodiment, the invention relates to a wheat plant, wheat seed or parts of a wheat plant comprising a polynucleotide of the SB Ella gene in the B genome with one or more non-transgenic mutations listed in Table 2 and corresponding to SEQ ID NO: 3. In another embodiment, the wheat plant, wheat seed or parts of a wheat plant comprises a polynucleotide with one or more non-transgenic mutations listed in Table 2 is 85%, 86%, 87%, 88%, 89%, 90%, 91%, 92%, 93%, 94%, 95%, 96%, 97%, 98%, 99% or greater than 99% identical or similar to SEQ ID NO: 3.

In still another embodiment, wheat plant, wheat seed or parts of a wheat plant comprises a polynucleotide with one or more non-transgenic mutations listed in Table 2 and codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 4.

In one embodiment, the invention relates to a wheat plant, wheat seed or parts of a wheat plant comprising a polynucleotide of the SBEIIa gene of the D genome with one or more non-transgenic mutations listed in Table 3 and corresponding to SEQ ID NO: 5. In another embodiment, the wheat plant, wheat seed or parts of a wheat plant comprise a polynucleotide with one or more non-transgenic mutations listed in Table 3 and is 85%, 86%, 87%, 88%, 89%, 90%, 91%, 92%, 93%, 94%, 95%, 96%, 97%, 98%, 99% or greater than 99% identical or similar to SEQ ID NO: 5.

In still another embodiment, the wheat plant, wheat seed or parts of a wheat plant comprises a polynucleotide with one or more non-transgenic mutations listed in Table 3 and codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 6.

In still another embodiment, the invention relates to a wheat plant, wheat seed or parts of a wheat plant comprising one or more non-transgenic mutations in the SBEIIb gene. In another embodiment, the invention relates to a wheat plant, wheat seed or parts of a wheat plant comprising at least one non-transgenic mutation in the SBEIIb gene in each of two genomes. In still another embodiment, the invention relates to a wheat plant, wheat seed or parts of a wheat plant comprising at least one non-transgenic mutation in the SBEIIb gene in each of three genomes.

In one embodiment, the wheat plant, wheat seed or parts of a wheat plant comprises one or more non-transgenic mutations in both alleles of the SBEIIb gene. In one embodiment, the wheat plant, wheat seed or parts of a wheat plant comprises one or more non-transgenic mutations in both alleles of the SBEIIb gene of the A genome. In another embodiment, the non-transgenic mutations are identical in both alleles of the SBEIIb gene of the A genome.

In one embodiment, the wheat plant, wheat seed or parts of a wheat plant comprises one or more non-transgenic mutations in both alleles of the SBEIIb gene of the B genome. In another embodiment, the non-transgenic mutations are identical in both alleles of the SBEIIb gene of the B genome.

In one embodiment, the wheat plant, wheat seed or parts of a wheat plant comprises one or more non-transgenic mutations in both alleles of the SBEIIb gene of the D genome. In another embodiment, the non-transgenic mutations are identical in both alleles of the SBEIIb gene of the D genome.

In one embodiment, the invention relates to a wheat plant, wheat seed or parts of a wheat plant comprising a polynucleotide of the SBEIIb gene of the A genome with one or more non-transgenic mutations listed in Table 4 and corresponding to SEQ ID NO: 7. In another embodiment, the wheat plant, wheat seed or parts of a wheat plant comprises a polynucleotide with one or more non-transgenic mutations listed in Table 4 and is 85%, 86%, 87%, 88%, 89%, 90%, 91%, 92%, 93%, 94%, 95%, 96%, 97%, 98%, 99% or greater than 99% identical or similar to SEQ ID NO: 7.

In still another embodiment, the wheat plant, wheat seed or parts of a wheat plant comprise a polynucleotide with one or more non-transgenic mutations listed in Table 4 that codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 8.

In one embodiment, the invention relates to a wheat plant, wheat seed or parts of a wheat plant comprising a polynucleotide of the SBEIIb gene of the B genome with one or more non-transgenic mutations listed in Table 5 and corresponding to SEQ ID NO: 9. In another embodiment, the wheat plant, wheat seed or parts of a wheat plant comprise a polynucleotide with one or more non-transgenic mutations listed in Table 5 and is 85%, 86%, 87%, 88%, 89%, 90%, 91%, 92%, 93%, 94%, 95%, 96%, 97%, 98%, 99% or greater than 99% identical or similar to SEQ ID NO: 9.

In still another embodiment, the wheat plant, wheat seed or parts of a wheat plant comprise a polynucleotide with one or more non-transgenic mutations listed in Table 5 that codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 10.

In one embodiment, the invention relates to wheat plant, wheat seed or parts of a wheat plant comprising a polynucleotide of the SBEIIb gene of the D genome with one or more non-transgenic mutations listed in Table 6 and corresponding to SEQ ID NO: 11. In another embodiment, the wheat plant, wheat seed or parts of a wheat plant comprise a polynucleotide with one or more non-transgenic mutations listed in Table 6 and is 85%, 86%, 87%, 88%, 89%, 90%, 91%, 92%, 93%, 94%, 95%, 96%, 97%, 98%, 99% or greater than 99% identical or similar to SEQ ID NO: 11.

In still another embodiment, the wheat plant, wheat seed or parts of a wheat plant comprise a polynucleotide with one or more non-transgenic mutations listed in Table 6 that codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 12.

In another embodiment, the invention relates to a wheat plant, wheat seed or parts of a wheat plant comprising one or more non-transgenic mutations in the SBEIIa and SBEIIb genes. In another embodiment, the invention relates to a wheat plant, wheat seed or parts of a wheat plant comprising at least one non-transgenic mutation in the SBEIIa and SBEIIb genes in each of two genomes. In still another embodiment, the invention relates to a wheat plant, wheat seed or parts of a wheat plant comprising at least one non-transgenic mutation in the SB Ella and SBEIIb genes in each of three genomes.

In still another embodiment, the invention relates to a wheat plant, wheat seed or parts of a wheat plant comprising at least one non-transgenic mutation in the SBEIIa gene in each of three genomes and one or more non-transgenic mutation in the SBEIIb gene.

In another embodiment, the wheat plant, wheat seed or parts of a wheat plant has one or more mutations in the SBEII gene including but not limited to one or more mutations enumerated in Tables 1-6 and 8-12 and corresponding mutations in the homoeologues. A wheat plant, wheat seed or parts of a wheat plant can be generated having 1, 2, 3, 4, 5, 6, 7, 8, 9, 10, 11, 12, 13, 14, 15, 16, 17, 18,19, 20, 21, 22, 23, 24, 25 or greater than 25 of the mutations disclosed herein including but not limited to the mutations disclosed in Tables 1-6 and 8-12, as well as mutations in the corresponding homoeologues.

In another embodiment, a wheat plant, wheat seed or parts of a wheat plant comprising one or more non-transgenic mutations in an SBEII gene, including but not limited to the mutation listed in Tables 1-6 and 8-12 and the mutations in the corresponding homoeologues, has an increased proportion of amylose in starch as compared to the same wheat cultivar without the mutations in the SBEII gene. In yet another embodiment, the proportion of amylose in the starch is selected from the group consisting of at least 10-15%, 16-20%, 21-25%, 26-30%, 31-35%, 36-40%, 41-45%, 46-50%, 51-55%, 56-60%, 61-65%, 66-70%, 71-75%, 76-80%, 81-85%, 86-90%, 91-95%, 96%, 97%, 98%, 99%, and greater than 99% (w/w).

### Grain, Flour and Starch

In another embodiment, the invention relates to a wheat grain, flour or starch comprising one or more non-transgenic mutations in the SBEII gene. In another embodiment, the invention relates to wheat grain comprising an embryo, wherein the embryo comprises one or more non-transgenic mutations in an SBEII gene.

In another embodiment, the wheat grain, flour or starch comprises one or more non-transgenic mutations in the SB Ella and/or the SBEIIb genes including but not limited to the mutations recited in Tables 1-6 and 8-12 and the corresponding mutations in homoeologues.

In still another embodiment, the invention relates to a wheat grain, flour or starch comprising one or more non-transgenic mutations in the SBEIIa gene. In another embodiment, the invention relates to a wheat grain or flour comprising at least one non-transgenic mutation in the SBEIIa gene in each of two genomes. In still another embodiment, the invention relates to a wheat grain or flour comprising at least one non-transgenic mutation in the SBEIIa gene in each of three genomes.

In one embodiment, the wheat grain, flour or starch comprises one or more non-transgenic mutations in both alleles of the SBEIIa gene in the A genome. In another embodiment, the non-transgenic mutations are identical in both alleles of the SBEIIa gene of the A genome.

In one embodiment, the wheat grain, flour or starch comprises one or more non-transgenic mutations in both alleles of the SBEIIa gene in the B genome. In another embodiment, the non-transgenic mutations are identical in both alleles of the SBEIIa gene of the B genome.

In one embodiment, the wheat grain, flour or starch comprises one or more non-transgenic mutations in both alleles of the SBEIIa gene in the D genome. In another embodiment, the non-transgenic mutations are identical in both alleles of the SBEIIa gene of the D genome.

In one embodiment, the invention relates to wheat grain, wheat flour or starch comprising a polynucleotide of the SBEIIa gene in the A genome with one or more non-transgenic mutations listed in Table 1 and corresponding to SEQ ID NO: 1. In another embodiment, the wheat grain or wheat flour comprise a polynucleotide with one or more non-transgenic mutations listed in Table 1 and is 85%, 86%, 87%, 88%, 89%, 90%, 91%, 92%, 93%, 94%, 95%, 96%, 97%, 98%, 99% or greater than 99% identical or similar to SEQ ID NO: 1.

In still another embodiment, wheat grain, wheat flour or starch comprise a polynucleotide with one or more non-transgenic mutations listed in Table 1 that codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 2.

In one embodiment, the invention relates to wheat grain, wheat flour or starch comprising a polynucleotide of the SBEIIa gene in the B genome with one or more non-transgenic mutations listed in Table 2 and corresponding to SEQ ID NO: 3. In another embodiment, the wheat grain or wheat flour comprises a polynucleotide with one or more non-transgenic mutations listed in Table 2 is 85%, 86%, 87%, 88%, 89%, 90%, 91%, 92%, 93%, 94%, 95%, 96%, 97%, 98%, 99% or greater than 99% identical or similar to SEQ ID NO: 3.

In still another embodiment, wheat grain, wheat flour or starch comprise a polynucleotide with one or more non-transgenic mutations listed in Table 2 and codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 4.

In one embodiment, the invention relates to wheat grain, wheat flour or starch comprising a polynucleotide of the SBEIIa gene of the D genome with one or more non-transgenic mutations listed in Table 3 and corresponding to SEQ ID NO: 5. In another embodiment, the wheat grain or wheat flour comprise a polynucleotide with one or more non-transgenic mutations listed in Table 3 and is 85%, 86%, 87%, 88%, 89%, 90%, 91%, 92%, 93%, 94%, 95%, 96%, 97%, 98%, 99% or greater than 99% identical or similar to SEQ ID NO: 5.

In still another embodiment, wheat grain, wheat flour or starch comprise a polynucleotide with one or more non-transgenic mutations listed in Table 3 and codes for a SBEIIa protein, wherein the SBEIIa protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 6.

In still another embodiment, the invention relates to a wheat grain, flour or starch comprising one or more non-transgenic mutations in the SBEIIb gene. In another embodiment, the invention relates to a wheat plant comprising at least one non-transgenic mutation in the SBEIIb gene in each of two genomes. In still another embodiment, the invention relates to a wheat plant comprising at least one non-transgenic mutation in the SBEIIb gene in each of three genomes.

In one embodiment, the wheat grain, flour or starch comprises one or more non-transgenic mutations in both alleles of the SBEIIb gene. In one embodiment, the wheat grain, flour or starch comprises one or more non-transgenic mutations in both alleles of the SBEIIb gene in each of two genomes. In one embodiment, the wheat grain, flour or starch comprises one or more non-transgenic mutations in both alleles of the SBEIIb gene in each of three genomes.

In one embodiment, the wheat grain, flour or starch comprises one or more non-transgenic mutations in both alleles of the SBEIIb gene. In one embodiment, the wheat grain, flour or starch comprises one or more non-transgenic mutations in both alleles of the SBEIIb gene of the A genome. In another embodiment, the non-transgenic mutations are identical in both alleles of the SBEIIb gene of the A genome.

In one embodiment, the wheat grain, flour or starch comprises one or more non-transgenic mutations in both alleles of the SBEIIb gene of the B genome. In another embodiment, the non-transgenic mutations are identical in both alleles of the SBEIIb gene of the B genome.

In one embodiment, the wheat grain, flour or starch comprises one or more non-transgenic mutations in both alleles of the SBEIIb gene of the D genome. In another embodiment, the non-transgenic mutations are identical in both alleles of the SBEIIb gene of the D genome.

In one embodiment, the invention relates to a wheat grain, wheat flour or starch comprising a polynucleotide of the SBEIIb gene of the A genome with one or more non-transgenic mutations listed in Table 4 and corresponding to SEQ ID NO: 7. In another embodiment, the wheat grain, wheat flour or starch comprises a polynucleotide with one or more non-transgenic mutations listed in Table 4 and is 85%, 86%, 87%, 88%, 89%, 90%, 91%, 92%, 93%, 94%, 95%, 96%, 97%, 98%, 99% or greater than 99% identical or similar to SEQ ID NO: 7.

In still another embodiment, the wheat grain, wheat flour or starch comprise a polynucleotide with one or more non-transgenic mutations listed in Table 4 that codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 8.

In one embodiment, the invention relates to wheat grain, wheat flour or starch comprising a polynucleotide of the SBEIIb gene of the B genome with one or more non-transgenic mutations listed in Table 5 and corresponding to SEQ ID NO: 9. In another embodiment, the wheat grain, wheat flour or starch comprise a polynucleotide with one or more non-transgenic mutations listed in Table 5 and is 85%, 86%, 87%, 88%, 89%, 90%, 91%, 92%, 93%, 94%, 95%, 96%, 97%, 98%, 99% or greater than 99% identical or similar to SEQ ID NO: 9.

In still another embodiment, the wheat grain, wheat flour or starch comprise a polynucleotide with one or more non-transgenic mutations listed in Table 5 that codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 10.

In one embodiment, the invention relates to wheat grain, wheat flour or starch comprising a polynucleotide of the SBEIIb gene of the D genome with one or more non-transgenic mutations listed in Table 6 and corresponding to SEQ ID NO: 11. In another embodiment, the wheat grain, wheat flour or starch comprise a polynucleotide with one or more non-transgenic mutations listed in Table 6 and is 85%, 86%, 87%, 88%, 89%, 90%, 91%, 92%, 93%, 94%, 95%, 96%, 97%, 98%, 99% or greater than 99% identical or similar to SEQ ID NO: 11.

In still another embodiment, the wheat grain, wheat flour or starch comprise a polynucleotide with one or more non-transgenic mutations listed in Table 6 that codes for a SBEIIb protein, wherein the SBEIIb protein comprises one or more non-transgenic mutations and is 85%, 86%, 87%, 88%, 89%, 90%, 91%, 92%, 93%, 94%, 95%, 96%, 97%, 98%, 99% or greater than 99% identical or similar to SEQ ID NO: 12.

In another embodiment, the invention relates to a wheat grain, flour or starch comprising one or more non-transgenic mutations in the SBEIIa gene and one or more non-transgenic mutations in the SBEIIb genes. In another embodiment, the invention relates to a wheat grain, flour or starch comprising at least one non-transgenic mutation in the SB Ella and SBEIIb genes in each of two genomes. In still another embodiment, the invention relates to a wheat grain, flour or starch comprising at least one non-transgenic mutation in the SB Ella and SBEIIb genes in each of three genomes.

In still another embodiment, the invention relates to a wheat grain, flour or starch comprising at least one non-transgenic mutation in the SB Ella gene in each of three genomes and one or more non-transgenic mutation in the SBEIIb gene.

In one embodiment, the wheat grain, flour or starch comprises one or more non-transgenic mutations in both alleles of the SBEIIa gene and the SBEIIb gene of the A genome.

In another embodiment, the non-transgenic mutations are identical in both alleles of the SBEIIa gene and the SBEIIb gene of the A genome.

In one embodiment, the wheat grain, flour or starch comprises one or more non-transgenic mutations in both alleles of the SBEIIa gene and the SBEIIb gene of the B genome.

In another embodiment, the non-transgenic mutations are identical in both alleles of the SBEIIa gene and the SBEIIb gene of the B genome.

In one embodiment, the wheat grain, flour or starch comprises one or more non-transgenic mutations in both alleles of the SBEIIa gene and the SBEIIb gene of the D genome.

In another embodiment, the non-transgenic mutations are identical in both alleles of the SBEIIa gene and the SBEIIb gene of the D genome.

In still another embodiment, the invention relates to wheat grain or flour comprising an endosperm and a reduced gene expression level, activity or expression level and activity of the SBEII gene as compared to wild type wheat grain or flour.

In still another embodiment, the invention relates to wheat grain or flour comprising an endosperm and a reduced expression level, activity or expression level and activity of the SBEII protein as compared to wild type wheat grain or flour. In still another embodiment, the invention relates to wheat grain or flour comprising an endosperm and a reduced expression level, activity or expression level and activity of the SBEIIa protein as compared to wild type wheat grain or flour. In yet another embodiment, the invention relates to wheat grain or flour comprising an endosperm and a reduced expression level, activity or expression level and activity of the SBEIIb protein as compared to wild type wheat grain or flour.

In yet another embodiment, the invention relates to wheat grain or flour comprising an altered starch component as compared to starch from wild type wheat grain or flour. In another embodiment, the wheat grain or flour comprises starch with a percentage of amylose selected from the group consisting of: 25-30%, 30-35%, 35-40%, 45-50%, 50-55%, 55-60%, 60-65%, 65-70%, 70-75%, 75-80%, 80-85%, 85-90%, 90-95%, and greater than 95% as compared to wild type grain or flour.

### Food Products

In one embodiment, the invention is directed to a flour or other product produced from the grain or flour discussed above. In another embodiments, the flour, the coarse fraction or purified starch may be a component of a food product.

The food product includes but is not limited to a bagel, a biscuit, a bread, a bun, a croissant, a dumpling, an English muffin, a muffin, a pita bread, a quickbread, a refrigerated/frozen dough products, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food.

In one embodiment, the flour is a whole grain flour (ex.—an ultrafine-milled whole grain flour, such as an ultrafine-milled whole grain wheat flour). In one embodiment, the whole grain flour includes a refined flour constituent (ex.—refined wheat flour or refined flour) and a coarse fraction (ex.—an ultrafine-milled coarse fraction). Refined wheat flour may be flour which is prepared, for example, by grinding and bolting (sifting) cleaned wheat. The Food and Drug Administration (FDA) requires flour to meet certain particle size standards in order to be included in the category of refined wheat flour. The particle size of refined wheat flour is described as flour in which not less than 98% passes through a cloth having openings not larger than those of woven wire cloth designated “212 micrometers (U.S. Wire 70).”

In another embodiment, the coarse fraction includes at least one of: bran and germ. For instance, the germ is an embryonic plant found within the wheat kernel. The germ includes lipids, fiber, vitamins, protein, minerals and phytonutrients, such as flavonoids. The bran may include several cell layers and has a significant amount of lipids, fiber, vitamins, protein, minerals and phytonutrients, such as flavonoids.

For example, the coarse fraction or whole grain flour or refined flour of the present invention may be used in various amounts to replace refined or whole grain flour in baked goods, snack products, and food products. The whole grain flour (i.e.—ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In another embodiment, the whole grain flour or coarse fraction or refined flour may be a component of a nutritional supplement. The nutritional supplement may be a product that is added to the diet containing one or more ingredients, typically including: vitamins, minerals, herbs, amino acids, enzymes, antioxidants, herbs, spices, probiotics, extracts, prebiotics and fiber.

In a further embodiment, the nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients.

Because of the high nutritional content of the endosperm of the present invention, there may be many uses that confer numerous benefits to an individual, including, delivery of fiber and other essential nutrients, increased digestive function and health, weight management, blood sugar management, heart health, diabetes risk reduction, potential arthritis risk reduction, and overall health and wellness for an individual.

In still another embodiments, the whole grain flour or coarse fraction or refined flour may be a component of a dietary supplement. The Code of Federal Regulations defines a dietary supplement as a product that is intended to supplement the diet and contains one or more dietary ingredients including: vitamins, minerals, herbs, botanicals, amino acids, and other substances or their constituents; is intended to be taken by mouth as a pill, capsule, tablet, or liquid; and is labeled on the front panel as being a dietary supplement.

In yet another embodiment, the whole grain flour or coarse fraction or refined flour may be a fiber supplement or a component thereof. The fiber supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage.

In another embodiment, the whole grain flour or coarse fraction or refined flour may be included as a component of a digestive supplement. The whole grain flour or coarse fraction or refined flour may be a component of a digestive supplement alone or in combination with one or more prebiotic compounds and/or probiotic organisms. Prebiotic compounds are non-digestible food ingredients that may beneficially affect the host by selectively stimulating the growth and/or the activity of a limited number of microorganisms in the colon. Examples of prebiotic compounds within the scope of the invention, may include, but are not limited to: oligosaccharides and inulins.

Probiotics are microorganisms which, when administered in adequate amounts, confer a health benefit on the host. Probiotic organisms include, but are not limited to: Lactobacillus, Bifidobacteria, Escherichia, Clostridium, Lactococcus, Streptococcus, Enterococcus, and Saccharomyces.

In yet another embodiment, the whole grain flour or coarse fraction or refined flour may be included as a component of a functional food. The Institute of Food Technologists defines functional foods as, foods and food components that provide a health benefit beyond basic nutrition. This includes conventional foods, fortified, enriched, or enhanced foods, and dietary supplements. The whole grain flour and coarse fraction or refined flour include numerous vitamins and minerals, have high oxygen radical absorption capacities, and are high in fiber, making them ideally suited for use in/as a functional food.

In another embodiment, the whole grain flour or coarse fraction or refined flour may be used in medical foods. Medical food is defined as a food that is formulated to be consumed or administered entirely under the supervision of a physician and which is intended for the specific dietary management of a disease or condition for which distinctive nutritional requirements, based on recognized scientific principles, are established by medical evaluation. The nutrient contents and antioxidant capacities of the whole grain flour and coarse fraction or refined flour make them ideal for use in medical foods.

In yet another embodiment, the whole grain flour or coarse fraction or refined flour may also be used in pharmaceuticals. The whole grain flour and coarse fraction or refined flour are high in fiber and have a very fine granulation making them suitable for use as a carrier in pharmaceuticals.

In still another embodiment, delivery of the whole grain flour or coarse fraction or refined flour as a nutritional supplement, dietary supplement or digestive supplement is contemplated via delivery mechanisms where the whole grain flour or coarse fraction is the single ingredient or one of many nutritional ingredients. Examples of delivery mechanisms include but are not limited to: instant beverage mixes, ready-to-drink beverages, nutritional bars, wafers, cookies, crackers, gel shots, capsules, and chews.

In yet another embodiment, a milling process may be used to make a multi-wheat flour, or a multi-grain coarse fraction. In one embodiment, bran and germ from one type of wheat may be ground and blended with ground endosperm or whole grain wheat flour of another type of wheat. Alternatively bran and germ of one type of grain may be ground and blended with ground endosperm or whole grain flour of another type of grain.

In still another embodiment, bran and germ from a first type of wheat or grain may be blended with bran and germ from a second type of wheat or grain to produce a multi-grain coarse fraction. It is contemplated that the invention encompasses mixing any combination of one or more of bran, germ, endosperm, and whole grain flour of one or more grains. This multi-grain, multi-wheat approach may be used to make custom flour and capitalize on the qualities and nutritional contents of multiple types of grains or wheats to make one flour.

The whole grain flour of the invention may be produced via a variety of milling processes. One exemplary process involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like.

After grinding, the grain is discharged and conveyed to a sifter. Any sifter known in the art for sifting a ground particle may be used. Material passing through the screen of the sifter is the whole grain flour of the invention and requires no further processing. Material that remains on the screen is referred to as a second fraction. The second fraction requires additional particle reduction. Thus, this second fraction may be conveyed to a second passage grinder.

After grinding, the second fraction may be conveyed to a second sifter. Material passing through the screen of the second sifter is the whole grain flour. The material that remains on the screen is referred to as the fourth fraction and requires further processing to reduce the particle size. The fourth fraction on the screen of the second sifter is conveyed back into either the first passage grinder or the second passage grinder for further processing via a feedback loop.

It is contemplated that the whole grain flour, coarse fraction, purified starch and/or grain products of the invention may be produced by a number of milling processes known in the art.

### Plant Breeding

In another embodiment, this invention is directed to methods for plant breeding using wheat plants and plant parts with one or more non-transgenic mutations in the SBEII gene.

One such embodiment is the method of crossing wheat variety with one or more non-transgenic mutations in the SBEII gene with another variety of wheat to form a first generation population of F1 plants. The population of first generation F1 plants produced by this method is also an embodiment of the invention. This first generation population of F1 plants will comprise an essentially complete set of the alleles of wheat variety with one or more non-transgenic mutations in the SBEII gene. One of ordinary skill in the art can utilize either breeder books or molecular methods to identify a particular F1 plant produced using wheat variety with one or more non-transgenic mutations in the SBEII gene, and any such individual plant is also encompassed by this invention. These embodiments also cover use of transgenic or backcross conversions of wheat varieties with one or more mutations in the SBEII gene to produce first generation F1 plants.

In another embodiment, the invention relates to a method of developing a progeny wheat plant. A method of developing a progeny wheat plant comprises crossing a wheat variety with one or more non-transgenic mutations in the SBEII gene with a second wheat plant and performing a breeding method. A specific method for producing a line derived from wheat variety with one or more non-transgenic mutations in the SBEII gene is as follows.

One of ordinary skill in the art would cross wheat variety with one or more non-transgenic mutations in the SBEII gene with another variety of wheat, such as an elite variety. The F1 seed derived from this cross would be grown to form a homogeneous population. The F1 seed would contain one set of the alleles from wheat variety with one or more non-transgenic mutations in the SBEII gene and one set of the alleles from the other wheat variety.

The F1 genome would be made-up of 50% wheat variety with one or more non-transgenic mutations in the SBEII gene and 50% of the other elite variety. The F1 seed would be grown to form F2 seed. The F1 seed could be allowed to self, or bred with another wheat cultivar.

On average the F2 seed would have derived 50% of its alleles from wheat variety with one or more non-transgenic mutations in the SBEII gene and 50% from the other wheat variety, but various individual plants from the population would have a much greater percentage of their alleles derived from wheat variety with one or more non-transgenic mutations in the SBEII gene (Wang J. and R. Bernardo, 2000, Crop Sci. 40:659-665 and Bernardo, R. and A. L. Kahler, 2001, Theor. Appl. Genet. 102:986-992).

The F2 seed would be grown and selection of plants would be made based on visual observation and/or measurement of traits and/or marker assisted selection. The wheat variety with one or more non-transgenic mutations in the SBEII gene-derived progeny that exhibit one or more of the desired wheat variety with one or more non-transgenic mutations in the SBEII gene-derived traits would be selected and each plant would be harvested separately. This F3 seed from each plant would be grown in individual rows and allowed to self. Then selected rows or plants from the rows would be harvested and threshed individually. The selections would again be based on visual observation and/or measurements for desirable traits of the plants, such as one or more of the desirable wheat variety with one or more non-transgenic mutations in the SBEII gene-derived traits.

The process of growing and selection would be repeated any number of times until a homozygous wheat variety with one or more non-transgenic mutations in the SBEII gene-derived wheat plant is obtained. The homozygous wheat variety with one or more non-transgenic mutations in the SBEII gene-derived wheat plant would contain desirable traits derived from wheat variety with one or more non-transgenic mutations in the SBEII gene, some of which may not have been expressed by the other original wheat variety to which wheat variety with one or more non-transgenic mutations in the SBEII gene was crossed and some of which may have been expressed by both wheat varieties but now would be at a level equal to or greater than the level expressed in wheat variety with one or more non-transgenic mutations in the SBEII gene.

The breeding process, of crossing, selfing, and selection may be repeated to produce another population of wheat variety with one or more non-transgenic mutations in the SBEII gene-derived wheat plants with, on average, 25% of their genes derived from wheat variety with one or more non-transgenic mutations in the SBEII gene, but various individual plants from the population would have a much greater percentage of their alleles derived from wheat variety with one or more non-transgenic mutations in the SBEII gene. Another embodiment of the invention is a homozygous wheat variety with one or more non-transgenic mutations in the SBEII gene-derived wheat plant that has received wheat variety with one or more non-transgenic mutations in the SBEII gene-derived traits.

The invention is further described by the following paragraphs.

1. A polynucleotide encoding an SBEIIa polypeptide comprising a tryptophan to a stop mutation at an amino acid corresponding to amino acid position 436 of SEQ ID NO: 2.

2. The polynucleotide of paragraph 1, wherein the SBEIIa polypeptide further comprises an amino acid sequence having at least 95% identity or similarity to SEQ ID NO: 2.

3. The polynucleotide of any of paragraphs 1-2, wherein the SBEIIa polypeptide further comprises an amino acid sequence having at least 97% identity or similarity to SEQ ID NO: 2.

4. The polynucleotide of any of paragraphs 1-3, wherein the SBEIIa polypeptide further comprises an amino acid sequence having at least 99% identity or similarity to SEQ ID NO: 2.

5. The polynucleotide of any of paragraphs 1-4 comprising a guanine to adenine mutation at a nucleotide position corresponding to nucleotide position 5267 of SEQ ID NO: 1.

6. The polynucleotide of any of paragraphs 1-5 further comprising at least 95% identity or similarity to SEQ ID NO: 1.

7. The polynucleotide of any of paragraphs 1-6 further comprising at least 97% identity or similarity to SEQ ID NO: 1.

8. The polynucleotide of any of paragraphs 1-7 further comprising at least 99% identity or similarity to SEQ ID NO: 1.

9. A polypeptide comprising an amino acid sequence having at least 95% identity or similarity to SEQ ID NO:2, wherein the polypeptide further comprises a tryptophan to a stop mutation at amino acid position 436 of SEQ ID NO: 2.

10. The polypeptide of paragraph 9 further comprising an amino acid sequence having at least 97% sequence identity or similarity to SEQ ID NO:2.

11. The polypeptide of any of paragraphs 9-10 further comprising an amino acid sequence having at least 99% sequence identity or similarity to SEQ ID NO:2.

12. The polypeptide of any of paragraphs 9-11 further comprising an amino acid sequence of SEQ ID NO:2 with a tryptophan to a stop mutation at amino acid position 436 or a fragment thereof having starch branching enzyme activity.

13. The polypeptide of any of paragraphs 1-12 further comprising an amino acid sequence of SEQ ID NO:2 with a tryptophan to a stop mutation at amino acid position 436.

14. A polynucleotide encoding an SBEIIa polypeptide comprising a tryptophan to a stop mutation at an amino acid corresponding to amino acid position 436 of SEQ ID NO: 4.

15. The polynucleotide of paragraph 14, wherein the SB Ella polypeptide further comprises an amino acid sequence having at least 95% identity or similarity to SEQ ID NO: 4.

16. The polynucleotide of any of paragraphs 14-15, wherein the SBEIIa polypeptide further comprises an amino acid sequence having at least 97% identity or similarity to SEQ ID NO: 4.

17. The polynucleotide of any of paragraphs 14-16, wherein the SBEIIa polypeptide further comprises an amino acid sequence having at least 99% identity or similarity to SEQ ID NO: 4.

18. The polynucleotide of any of paragraphs 14-17 comprising a guanine to adenine mutation at a nucleotide position corresponding to nucleotide position 5038 of SEQ ID NO: 3.

19. The polynucleotide of any of paragraphs 14-18 further comprising at least 95% identity or similarity to SEQ ID NO: 3.

20. The polynucleotide of any of paragraphs 14-19 further comprising at least 97% identity or similarity to SEQ ID NO: 3.

21. The polynucleotide of any of paragraphs 14-20 further comprising at least 99% identity or similarity to SEQ ID NO: 3.

22. A polypeptide comprising an amino acid sequence having at least 95% identity or similarity to SEQ ID NO:4, wherein the polypeptide further comprises a tryptophan to a stop mutation at amino acid position 436 of SEQ ID NO: 4.

23. The polypeptide of paragraph 22 further comprising an amino acid sequence having at least 97% sequence identity or similarity to SEQ ID NO:4.

24. The polypeptide of any of paragraphs 22-23 further comprising an amino acid sequence having at least 99% sequence identity or similarity to SEQ ID NO:4.

25. The polypeptide of any of paragraphs 22-24 comprising an amino acid sequence of SEQ ID NO:4 with a tryptophan to a stop mutation at amino acid position 436 or a fragment thereof having starch branching enzyme activity.

26. The polypeptide of any of paragraphs 22-25 comprising an amino acid sequence of SEQ ID NO:4 with a tryptophan to a stop mutation at amino acid position 436.

27. A polynucleotide encoding an SBEIIa polypeptide comprising a tryptophan to a stop mutation at an amino acid corresponding to amino acid position 432 of SEQ ID NO: 6.

28. The polynucleotide of paragraph 27, wherein the SBEIIa polypeptide further comprises an amino acid sequence having at least 95% identity or similarity to SEQ ID NO: 6.

29. The polynucleotide of any of paragraphs 27-28, wherein the SBEIIa polypeptide further comprises an amino acid sequence having at least 97% identity or similarity to SEQ ID NO: 6.

30. The polynucleotide of any of paragraphs 27-29, wherein the SBEIIa polypeptide further comprises an amino acid sequence having at least 99% identity or similarity to SEQ ID NO: 6.

31. The polynucleotide of any of paragraphs 27-30 comprising a guanine to adenine mutation at a nucleotide position corresponding to nucleotide position 6305 of SEQ ID NO: 5.

32. The polynucleotide of any of paragraphs 27-31 further comprising at least 95% identity or similarity to SEQ ID NO: 5.

33. The polynucleotide of any of paragraphs 27-32 further comprising at least 97% identity or similarity to SEQ ID NO: 5.

34. The polynucleotide of any of paragraphs 27-33 further comprising at least 99% identity or similarity to SEQ ID NO: 5.

35. A polypeptide comprising an amino acid sequence having at least 95% identity or similarity to SEQ ID NO:6, wherein the polypeptide further comprises a tryptophan to a stop mutation at amino acid position 432 of SEQ ID NO: 6.

36. The polypeptide of paragraph 35 further comprising an amino acid sequence having at least 97% sequence identity or similarity to SEQ ID NO:6.

37. The polypeptide of any of paragraphs 35-36 further comprising an amino acid sequence having at least 99% sequence identity or similarity to SEQ ID NO:6.

38. The polypeptide of any of paragraphs 35-37 comprising an amino acid sequence of SEQ ID NO:6 with a tryptophan to a stop mutation at amino acid position 432 or a fragment thereof having starch branching enzyme activity.

39. The polypeptide of any of paragraphs 35-38 comprising an amino acid sequence of SEQ ID NO:6 with a tryptophan to a stop mutation at amino acid position 432.

40. A polynucleotide encoding an SBEIIa polypeptide comprising a tryptophan to a stop mutation at an amino acid corresponding to amino acid position 446 of SEQ ID NO: 4.

41. The polynucleotide of paragraph 40, wherein the SB Ella polypeptide further comprises an amino acid sequence having at least 95% identity or similarity to SEQ ID NO: 4.

42. The polynucleotide of any of paragraphs 40-41, wherein the SBEIIa polypeptide further comprises an amino acid sequence having at least 97% identity or similarity to SEQ ID NO: 4.

43. The polynucleotide of any of paragraphs 40-42, wherein the SBEIIa polypeptide further comprises an amino acid sequence having at least 99% identity or similarity to SEQ ID NO: 4.

44. The polynucleotide of any of paragraphs 40-43 comprising a guanine to adenine mutation at a nucleotide position corresponding to nucleotide position 5069 of SEQ ID NO: 3.

45. The polynucleotide of any of paragraphs 40-44 further comprising at least 95% identity or similarity to SEQ ID NO: 3.

46. The polynucleotide of any of paragraphs 40-45 further comprising at least 97% identity or similarity to SEQ ID NO: 3.

47. The polynucleotide of any of paragraphs 40-46 further comprising at least 99% identity or similarity to SEQ ID NO: 3.

48. A polypeptide comprising an amino acid sequence having at least 95% identity or similarity to SEQ ID NO:4, wherein the polypeptide further comprises a tryptophan to a stop mutation at amino acid position 446 of SEQ ID NO: 4.

49. The polypeptide of paragraph 48 further comprising an amino acid sequence having at least 97% sequence identity or similarity to SEQ ID NO:4.

50. The polypeptide of paragraphs 48-49 further comprising an amino acid sequence having at least 99% sequence identity or similarity to SEQ ID NO:4.

51. The polypeptide of any of paragraphs 48-50 comprising an amino acid sequence of SEQ ID NO:4 with a tryptophan to a stop mutation at amino acid position 446 or a fragment thereof having starch branching enzyme activity.

52. The polypeptide of any of paragraphs 48-51 comprising an amino acid sequence of SEQ ID NO:4 with a tryptophan to a stop mutation at amino acid position 446.

53. A n SBEIIa polynucleotide comprising a guanine to adenine mutation at a nucleotide position corresponding to nucleotide position 5073 of SEQ ID NO: 3.

54. The polynucleotide of paragraph 53 further comprising at least 95% identity or similarity to SEQ ID NO: 3.

55. The polynucleotide of any of paragraph 53-54 further comprising at least 97% identity or similarity to SEQ ID NO: 3.

56. The polynucleotide of any of paragraphs 53-55 further comprising at least 99% identity or similarity to SEQ ID NO: 3.

57. A polynucleotide encoding an SBEIIa polypeptide comprising a glycine to a glutamate mutation at an amino acid corresponding to amino acid position 467 of SEQ ID NO: 4.

58. The polynucleotide of paragraph 57, wherein the SBEIIa polypeptide further comprises an amino acid sequence having at least 95% identity or similarity to SEQ ID NO: 4.

59. The polynucleotide of any of paragraphs 57-58, wherein the SBEIIa polypeptide further comprises an amino acid sequence having at least 97% identity or similarity to SEQ ID NO: 4.

60. The polynucleotide of any of paragraphs 57-59, wherein the SBEIIa polypeptide further comprises an amino acid sequence having at least 99% identity or similarity to SEQ ID NO: 4.

61. The polynucleotide of any of paragraphs 57-60 comprising a guanine to adenine mutation at a nucleotide position corresponding to nucleotide position 5219 of SEQ ID NO: 3.

62. The polynucleotide of any of paragraphs 57-61 further comprising at least 95% identity or similarity to SEQ ID NO: 3.

63. The polynucleotide of any of paragraphs 57-62 further comprising at least 97% identity or similarity to SEQ ID NO: 3.

64. The polynucleotide of any of paragraphs 57-63 further comprising at least 99% identity or similarity to SEQ ID NO: 3.

65. A polypeptide comprising an amino acid sequence having at least 95% identity or similarity to SEQ ID NO:4, wherein the polypeptide further comprises a glycine to a glutamate mutation at amino acid position 467 of SEQ ID NO: 4.

66. The polypeptide of paragraph 65 further comprising an amino acid sequence having at least 97% sequence identity or similarity to SEQ ID NO:4.

67. The polypeptide of any of paragraphs 65-66 further comprising an amino acid sequence having at least 99% sequence identity or similarity to SEQ ID NO:4.

68. The polypeptide of any of paragraphs 65-67 comprising an amino acid sequence of SEQ ID NO:4 with a glycine to a glutamate mutation at amino acid position 467 or a fragment thereof having starch branching enzyme activity.

69. The polypeptide of any of paragraphs 65-68 comprising an amino acid sequence of SEQ ID NO:4 with a glycine to a glutamate mutation at amino acid position 467.

70. A polynucleotide encoding an SBEIIa polypeptide comprising a tryptophan to a stop mutation at an amino acid corresponding to amino acid position 442 of SEQ ID NO: 6.

71. The polynucleotide of paragraph 70, wherein the SBEIIa polypeptide further comprises an amino acid sequence having at least 95% identity or similarity to SEQ ID NO: 6.

72. The polynucleotide of any of paragraphs 70-71, wherein the SBEIIa polypeptide further comprises an amino acid sequence having at least 97% identity or similarity to SEQ ID NO: 6.

73. The polynucleotide of any of paragraphs 70-72, wherein the SBEIIa polypeptide further comprises an amino acid sequence having at least 99% identity or similarity to SEQ ID NO: 6.

74. The polynucleotide of any of paragraphs 70-73 comprising a guanine to adenine mutation at a nucleotide position corresponding to nucleotide position 6335 of SEQ ID NO: 5.

75. The polynucleotide of any of paragraphs 70-74 further comprising at least 95% identity or similarity to SEQ ID NO: 5.

76. The polynucleotide of any of paragraphs 70-75 further comprising at least 97% identity or similarity to SEQ ID NO: 5.

77. The polynucleotide of any of paragraphs 70-76 further comprising at least 99% identity or similarity to SEQ ID NO: 5.

78. A polypeptide comprising an amino acid sequence having at least 95% identity or similarity to SEQ ID NO:6, wherein the polypeptide further comprises a tryptophan to a stop mutation at amino acid position 442 of SEQ ID NO: 6.

79. The polypeptide of paragraph 78 further comprising an amino acid sequence having at least 97% sequence identity or similarity to SEQ ID NO:6.

80. The polypeptide of any of paragraphs 78-79 further comprising an amino acid sequence having at least 99% sequence identity or similarity to SEQ ID NO:6.

81. The polypeptide of any of paragraphs 78-80 further comprising an amino acid sequence of SEQ ID NO:6 with a tryptophan to a stop mutation at amino acid position 442 or a fragment thereof having starch branching enzyme activity.

82. The polypeptide of any of paragraphs 78-81 comprising an amino acid sequence of SEQ ID NO:6 with a tryptophan to a stop mutation at amino acid position 442.

83. A polynucleotide encoding an SBEIIb polypeptide comprising a tryptophan to a stop mutation at an amino acid corresponding to amino acid position 285 of SEQ ID NO: 8.

84. The polynucleotide of paragraph 83, wherein the SBEIIb polypeptide further comprises an amino acid sequence having at least 95% identity or similarity to SEQ ID NO: 8.

85. The polynucleotide of any of paragraphs 83-84, wherein the SBEIIb polypeptide further comprises an amino acid sequence having at least 97% identity or similarity to SEQ ID NO: 8.

86. The polynucleotide of any of paragraphs 83-85, wherein the SBEIIb polypeptide further comprises an amino acid sequence having at least 99% identity or similarity to SEQ ID NO: 8.

87. The polynucleotide of any of paragraphs 83-86 comprising a guanine to adenine mutation at a nucleotide position corresponding to nucleotide position 2282 of SEQ ID NO: 7.

88. The polynucleotide of any of paragraphs 83-87 further comprising at least 95% identity or similarity to SEQ ID NO: 7.

89. The polynucleotide of any of paragraphs 83-88 further comprising at least 97% identity or similarity to SEQ ID NO: 7.

90. The polynucleotide of any of paragraphs 83-89 further comprising at least 99% identity or similarity to SEQ ID NO: 7.

91. A polypeptide comprising an amino acid sequence having at least 95% identity or similarity to SEQ ID NO:8, wherein the polypeptide further comprises a tryptophan to a stop mutation at amino acid position 285 of SEQ ID NO: 8.

92. The polypeptide of paragraph 91 further comprising an amino acid sequence having at least 97% sequence identity or similarity to SEQ ID NO:8.

93. The polypeptide of any of paragraphs 91-92 further comprising an amino acid sequence having at least 99% sequence identity or similarity to SEQ ID NO:8.

94. The polypeptide of any of paragraphs 91-93 further comprising an amino acid sequence of SEQ ID NO:8 with a tryptophan to a stop mutation at amino acid position 285 or a fragment thereof having starch branching enzyme activity.

95. The polypeptide of any of paragraphs 91-94 comprising an amino acid sequence of SEQ ID NO:8 with a tryptophan to a stop mutation at amino acid position 285.

96. A wheat plant comprising a polynucleotide of any of paragraphs 1-8, 14-21, 27-34,40-47, 53-56, 57-64, 70-77, and 83-90.

97. A wheat plant comprising at least two non-transgenic mutations in an SBEII gene, wherein at least one mutation is in the SBEIIa gene as recited in any of paragraphs 1-8, 14-21, 27-34, 40-47, 53-56, 57-64, and 70-77.

98. The wheat plant of any of paragraphs 96-97, wherein a second non-transgenic mutation is in the SBEIIb gene. The SBEIIb mutations may be as recited in paragraphs 83-90.

99. The wheat plant of any of paragraphs 96-98, wherein the first and second mutations are in the SBEIIa gene.

100. The wheat plant of any of paragraphs 96-99, wherein the first and second mutations are in the same genome.

101. The wheat plant of any of paragraphs 96-100, wherein the first and second mutations are in different genomes.

102. The wheat plant of any of paragraphs 96-101, further comprising at least three non-transgenic mutations in the SBEII gene.

103. The wheat plant of any of paragraphs 96-102, wherein two mutations are in the same genome.

104. The wheat plant of any of paragraphs 96-103, wherein three mutations are in different genomes.

105. The wheat plant of any of paragraphs 96-104, wherein the three mutations are in each of the A genome, B genome and D genome. A ny number of mutations are possible including 1, 2, 3, 4, 5, 6, 7, 8, 9, 10, and greater than 10 mutations in the SBEIIa gene and including 1, 2, 3, 4, 5, 6, 7, 8, 9, 10, and greater than 10 mutations in the SBEIIb gene.

106. A wheat plant comprising at least two polynucleotides as recited in any of paragraphs 1-8, 14-21, 27-34, 40-47, 53-56, 57-64, 70-77, and 83-90

107. A wheat plant comprising a polypeptide of any of paragraphs 9-13, 22-26, 35-39, 48-52, 65-69, 78-82, and 91-95.

108. The wheat plant of any of paragraphs 96-107, wherein the wheat is diploid, tetraploid or hexaploid.

109. A hexaploid wheat plant comprising at least one mutation in each SB Ella gene, wherein the mutation in the SBEIIa gene of the A genome corresponds to a guanine to adenine mutation at nucleotide position 5267 of SEQ ID NO: 1, wherein the mutation in the SB Ella gene of the B genome corresponds to a guanine to adenine mutation at nucleotide position 5308 of SEQ ID NO: 3; and wherein the mutation in the SBEIIa gene of the D genome corresponds to a guanine to adenine mutation at nucleotide position 6305 of SEQ ID NO: 5.

110. A hexaploid wheat plant comprising at least one mutation in each SB Ella gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5069 of SEQ ID NO: 3; and wherein the mutation in the SBEIIa gene of the D genome corresponds to a guanine to adenine mutation at nucleotide position 6335 of SEQ ID NO: 5.

111. A hexaploid wheat plant comprising at least one mutation in each SB Ella gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5193 of SEQ ID NO: 3; and wherein the mutation in the SBEIIa gene of the D genome corresponds to a guanine to adenine mutation at nucleotide position 6305 of SEQ ID NO: 5.

112. A wheat plant comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5073 of SEQ ID NO: 3.

113. A wheat plant comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5219 of SEQ ID NO: 3.

114. A wheat plant comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5033 of SEQ ID NO: 3.

115. A wheat seed comprising a polynucleotide of any of paragraphs 1-8, 14-21, 27-34, 40-47, 53-56, 57-64, 70-77, and 83-90.

116. A wheat seed comprising at least two non-transgenic mutations in an SBEII gene, wherein at least one mutation is in the SBEIIa gene as recited in any of paragraphs 1-8, 14-21, 27-34, 40-47, 53-56, 57-64, 70-77, and 83-90.

117. The wheat seed of any of paragraphs 115-115, wherein a second non-transgenic mutation is in the SBEIIb gene.

118. The wheat seed of any of paragraphs 115-117, wherein the first and second mutations are in the SBEIIa gene.

119. The wheat seed of any of paragraphs 115-118, wherein the first and second mutations are in the same genome.

120. The wheat seed of any of paragraphs 115-119, wherein the first and second mutations are in different genomes.

121. The wheat seed of any of paragraphs 115-120 further comprising at least three non-transgenic mutations in the SBEII gene.

122. The wheat seed of any of paragraphs 115-121, wherein three mutations are in the same genome.

123. The wheat seed of any of paragraphs 115-122, wherein three mutations are in different genomes.

124. The wheat seed of any of paragraphs 115-123, wherein the three mutations are in each of the A genome, B genome and D genome.

125. A wheat seed comprising at least two polynucleotides as recited in any of paragraphs 1-8, 14-21, 27-34, 40-47, 53-56, 57-64, 70-77, and 83-90.

126. A wheat seed comprising a polypeptide of any of paragraphs 9-13, 22-26, 35-39, 48-52, 65-69, 78-82, and 91-95.

127. The wheat seed of any of paragraphs 115-126, wherein the wheat is diploid, tetraploid or hexaploid.

128. A hexaploid wheat seed comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5308 of SEQ ID NO: 3; and wherein the mutation in the SB Ella gene of the D genome corresponds to a guanine to adenine mutation at nucleotide position 6305 of SEQ ID NO: 5.

129. A hexaploid wheat seed comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5069 of SEQ ID NO: 3; and wherein the mutation in the SBEIIa gene of the D genome corresponds to a guanine to adenine mutation at nucleotide position 6335 of SEQ ID NO: 5.

130. A hexaploid wheat seed comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5193 of SEQ ID NO: 3; and wherein the mutation in the SBEIIa gene of the D genome corresponds to a guanine to adenine mutation at nucleotide position 6305 of SEQ ID NO: 5.

131. A wheat seed comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5073 of SEQ ID NO: 3.

132. A wheat seed comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5219 of SEQ ID NO: 3.

133. A wheat seed comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5033 of SEQ ID NO: 3.

134. Wheat grain comprising a polynucleotide of any of paragraphs 1-8, 14-21, 27-34, 40-47, 53-56, 57-64, 70-77, and 83-90.

135. Wheat grain comprising at least two non-transgenic mutations in an SBEII gene, wherein one mutation is in the SBEIIa gene as recited in any of paragraphs 1-8, 14-21, 27-34, 40-47, 53-56, 57-64, 70-77, and 83-90.

136. The wheat grain of any of paragraphs 134-135, wherein a second non-transgenic mutation is in the SBEIIb gene.

137. The wheat grain of any of paragraphs 134-136, wherein the first and second mutations are in the SBEIIa gene.

138. The wheat grain of any of paragraphs 134-137, wherein the first and second mutations are in the same genome.

139. The wheat grain of any of paragraphs 134-138, wherein the first and second mutations are in different genomes.

140. The wheat grain of any of paragraphs 134-139, further comprising at least three non-transgenic mutations in the SBEII gene.

141 The wheat grain of any of paragraphs 134-140, wherein the three mutations are in the same genome.

142. The wheat grain of any of paragraphs 134-141, wherein the three mutations are in different genomes.

143. The wheat grain of any of paragraphs 134-142, wherein the three mutations are in each of the A genome, B genome and D genome.

144. Wheat grain comprising at least two polynucleotides as recited in any of paragraphs 1-8, 14-21, 27-34, 40-47, 53-56, 57-64, 70-77, and 83-90.

145. Wheat grain comprising a polypeptide of any of paragraphs 9-13, 22-26, 35-39, 48-52, 65-69, 78-82, and 91-95.

146. Wheat grain of any of paragraphs 134-145, wherein the wheat is diploid, tetraploid or hexaploid.

147. A hexaploid wheat grain comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5308 of SEQ ID NO: 3; and wherein the mutation in the SB Ella gene of the D genome corresponds to a guanine to adenine mutation at nucleotide position 6305 of SEQ ID NO: 5.

148. A hexaploid wheat grain comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5069 of SEQ ID NO: 3; and wherein the mutation in the SBEIIa gene of the D genome corresponds to a guanine to adenine mutation at nucleotide position 6335 of SEQ ID NO: 5.

149. A hexaploid wheat grain comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5193 of SEQ ID NO: 3; and wherein the mutation in the SBEIIa gene of the D genome corresponds to a guanine to adenine mutation at nucleotide position 6305 of SEQ ID NO: 5.

150. A wheat grain comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5073 of SEQ ID NO: 3.

151. A wheat grain comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5219 of SEQ ID NO: 3.

152. A wheat grain comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5033 of SEQ ID NO: 3.

153. Wheat flour comprising a polynucleotide of any of paragraphs 1-8, 14-21, 27-34, 40-47, 53-56, 57-64, 70-77, and 83-90.

154. Wheat flour comprising at least two non-transgenic mutations in an SBEII gene, wherein one mutation is in the SBEIIa gene as recited in any of paragraphs 1-8, 14-21, 27-34, 40-47, 53-56, 57-64, 70-77, and 83-90.

155. The wheat flour of any of paragraphs 153-154, wherein a second non-transgenic mutation is in the SBEIIb gene.

156. The wheat flour of any of paragraphs 153-155, wherein the first and second mutations are in the SBEIIa gene.

157. The wheat flour of any of paragraphs 153-156, wherein the first and second mutations are in the same genome.

158. The wheat flour of any of paragraphs 153-157, wherein the first and second mutations are in different genomes.

159. The wheat flour of any of paragraphs 153-158, further comprising at least three non-transgenic mutations in the SBEII gene.

160. The wheat flour of any of paragraphs 153-159, wherein the three mutations are in the same genome.

161. The wheat flour of any of paragraphs 153-160, wherein the three mutations are in different genomes.

162. The wheat flour of any of paragraphs 153-161, wherein the three mutations are in each of the A genome, B genome and D genome.

163. Wheat flour comprising at least two polynucleotides as recited in any of paragraphs 1-8, 14-21, 27-34, 40-47, 53-56, 57-64, 70-77, and 83-90.

164. Wheat flour comprising a polypeptide of any of paragraphs 9-13, 22-26, 35-39, 48-52, 65-69, 78-82, and 91-95.

165. Wheat flour of any of paragraphs 153-164, wherein the wheat is diploid, tetraploid or hexaploid.

166. A hexaploid wheat flour comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5308 of SEQ ID NO: 3; and wherein the mutation in the SB Ella gene of the D genome corresponds to a guanine to adenine mutation at nucleotide position 6305 of SEQ ID NO: 5.

167. A hexaploid wheat flour comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5069 of SEQ ID NO: 3; and wherein the mutation in the SBEIIa gene of the D genome corresponds to a guanine to adenine mutation at nucleotide position 6335 of SEQ ID NO: 5.

168. A hexaploid wheat flour comprising at least one mutation in each SBEIIa gene, wherein the mutation in the SB Ella gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5193 of SEQ ID NO: 3; and wherein the mutation in the SBEIIa gene of the D genome corresponds to a guanine to adenine mutation at nucleotide position 6305 of SEQ ID NO: 5.

169. A wheat flour comprising at least one mutation in each SB Ella gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5073 of SEQ ID NO: 3.

170. A wheat flour comprising at least one mutation in each SB Ella gene, wherein the mutation in the SBEIIa gene of the A genome corresponds to a guanine to adenine mutation at nucleotide position 5267 of SEQ ID NO: 1, wherein the mutation in the SB Ella gene of the B genome corresponds to a guanine to adenine mutation at nucleotide position 5219 of SEQ ID NO: 3.

171. A wheat flour comprising at least one mutation in each SBEIIa gene, wherein the mutation in the SBEIIa gene of the A genome corresponds to a guanine to adenine mutation at nucleotide position 5267 of SEQ ID NO: 1, wherein the mutation in the SBEIIa gene of the B genome corresponds to a guanine to adenine mutation at nucleotide position 5033 of SEQ ID NO: 3.

172. A food product comprising the wheat grain of any of paragraphs 134-152.

173. A food product comprising the wheat flour of any of paragraphs 153-171.

174. Use of a polynucleotide according to any of paragraphs 1-8, 14-21, 27-34, 40-47, 53-56, 57-64, 70-77, and 83-90 for the production of wheat having increased amylose levels compared to wild type wheat, wherein said polynucleotide contributes to the increased amylose levels.

175. Use of a polynucleotide according to any of paragraphs 1-8, 14-21, 27-34, 40-47, 53-56, 57-64, 70-77, and 83-90 for the selection of wheat having increased amylose levels compared to wild type wheat, wherein genomic DNA is isolated from the wheat and a segment of said SBEII gene is amplified and the presence of said gene is detected.

176. Use of a polypeptide according to any of paragraphs 9-13, 22-26, 35-39, 48-52, 65-69, 78-82, and 91-95 for the production of wheat having increased amylose levels compared to wild type wheat, wherein said polynucleotide contributes to the increased amylose levels.

177. Use of a polypeptide according to any of paragraphs 9-13, 22-26, 35-39, 48-52, 65-69, 78-82, and 91-95 for the selection of wheat having increased amylose levels compared to wild type wheat, wherein genomic DNA is isolated from the wheat and a segment of said SB EI I gene is amplified and the presence of said gene is detected.

### Example 1

### Mutagenesis

In accordance with one exemplary embodiment of the present invention, wheat seeds of the hexaploid cultivar (Triticum aestivum) Express and of the tetraploid cultivar (Triticum turgidum, Durum) Kronos were vacuum infiltrated in H₂O (approximately 1,000 seeds/100 ml H₂O for approximately 4 minutes). The seeds were then placed on a shaker (45 rpm) in a fume hood at room temperature. The mutagen ethyl methanesulfonate (EMS) was added to the imbibing seeds to final concentrations ranging from about 0.75% to about 1.2% (v/v). Following an 18-hour incubation period, the EMS solution was replaced 4 times with fresh H₂O. The seeds were then rinsed under running water for about 4-8 hours. Finally, the mutagenized seeds were planted (96/tray) in potting soil and allowed to germinate indoors. Plants that were four to six weeks old were transferred to the field to grow to fully mature M1 plants. The mature M1 plants were allowed to self-pollinate and then seeds from the M1 plant were collected and planted to produce M2 plants.

### DNA Preparation

DNA from the M2 plants produced in accordance with the above description was extracted and prepared in order to identify which M2 plants carried a mutation at one or more of their SBEII loci. The M2 plant DNA was prepared using the methods and reagents contained in the Qiagen® (Valencia, CA) DNeasy® 96 Plant Kit. Approximately 50 mg of frozen plant sample was placed in a sample tube with a tungsten bead, frozen in liquid nitrogen and ground 2 times for 1 minute each at 20 Hz using the Retsch® Mixer Mill MM 300. Next, 400 μl of solution AP1 [BufferAP1, solution DX and RNA se (100 mg/ml)] at 80° C. was added to the sample. The tube was sealed and shaken for 15 seconds. Following the addition of 130 μl Buffer AP2, the tube was shaken for 15 seconds. The samples were placed in a freezer at minus 20° C. for at least 1 hour. The samples were then centrifuged for 20 minutes at 5,600×g. A 400 μl aliquot of supernatant was transferred to another sample tube. Following the addition of 600 μl of Buffer AP3/E, this sample tube was capped and shaken for 15 seconds. A filter plate was placed on a square well block and 1 ml of the sample solution was applied to each well and the plate was sealed. The plate and block were centrifuged for 4 minutes at 5,600×g. Next, 800 μl of Buffer AW was added to each well of the filter plate, sealed and spun for 15 minutes at 5,600×g in the square well block. The filter plate was then placed on a new set of sample tubes and 80 μl of Buffer AE was applied to the filter. It was capped and incubated at room temperature for 1 minute and then spun for 2 minutes at 5600×g. This step was repeated with an additional 80 μl Buffer AE. The filter plate was removed and the tubes containing the pooled filtrates were capped. The individual samples were then normalized to a DNA concentration of 5 to 10 ng/μl.

### Tilling

The M2 DNA was pooled into groups of two individual plants. The DNA concentration for each individual within the pool was approximately 0.8 ng/μl with a final concentration of 1.6 ng/μl for the entire pool. Then, 5 μl of the pooled DNA samples (or 8 ng wheat DNA) was arrayed on microtiter plates and subjected to gene-specific PCR.

PCR amplification was performed in 15 μl volumes containing 2.5 ng pooled DNA, 0.75×ExTaq buffer (Panvera®, Madison, WI), 2.6 mM MgCl₂, 0.3 mM dNTPs, 0.3 μM primers, and 0.05 U Ex-Taq (Panvera®) DNA polymerase. PCR amplification was performed using an MJ Research® thermal cycler as follows: 95° C. for 2 minutes; 8 cycles of “touchdown PCR” (94° C. for 20 second, followed by annealing step starting at 70-68° C. for 30 seconds and decreasing 1° C. per cycle, then a temperature ramp of 0.5° C. per second to 72° C. followed by 72° C. for 1 minute); 25-45 cycles of 94° C. for 20 seconds, 63-61° C. for 30 seconds, ramp 0.5° C./sec to 72° C., 72° C. for 1 minute; 72° C. for 8 minutes; 98° C. for 8 minutes; 80° C. for 20 seconds; 60 cycles of 80° C. for 7 seconds-0.3 degrees/cycle.

The PCR primers (MWG Biotech, Inc., High Point, NC) were mixed as follows:
2.5 μl 100 μM IRD-700 labeled left primer
7.5 μl 100 μM left primer
9.0 μl 100 μM IRD-800 labeled right primer
1.0 μl 100 μM right primer

A label can be attached to each primer as described or to only one of the primers. Alternatively, Cy5.5 modified primers could be used. The label was coupled to the oligonucleotide using conventional phosphoramidite chemistry.

PCR products (15 μl) were digested in 96-well plates. Next, 30 μl of a solution containing 10 mM H EPES [4-(2-hydroxyethyl)-1-piperazineethanesulfonic acid](pH 7.5), 10 mM MgSO₄, 0.002% (w/v) Triton® X-100, 20 ng/ml of bovine serum albumin, and Surveyor® endonuclease (Transgenomic®, Inc.; 1:100,000 dilution) was added with mixing on ice, and the plate was incubated at 45° C. for 15 minutes. The specific activity of the Surveyor enzyme was 800 units/μl, where a unit was defined by the manufacturer as the amount of enzyme required to produce 1 ng of acid-soluble material from sheared, heat denatured calf thymus DNA at pH 8.5 in one minute at 37° C. Reactions were stopped by addition of 10 μl of a 2.5 M NaCl solution with 0.5 mg/ml blue dextran and 75 mM E DTA, followed by the addition of 80 μl isopropanol. The reactions were precipitated at room temperature, spun at 4,000 rpm for 30 minutes in an Eppendorf Centrifuge 5810. Pellets were resuspended in 8 μl of 33% formamide with 0.017% bromophenol blue dye, heated at 80° C. for 7 minutes and then at 95° C. for 2 minutes. Samples were transferred to a membrane comb using a comb-loading robot (MWG Biotech). The comb was inserted into a slab acrylamide gel (6.5%), electrophoresed for 10 min, and removed. Electrophoresis was continued for 4 hours at 1,500-V, 40-W, and 40-mA limits at 50° C.

During electrophoresis, the gel was imaged using a LI-COR© (Lincoln, NE) scanner which was set at a channel capable of detecting the IR Dye 700 and 800 labels. The gel image showed sequence-specific pattern of background bands common to all 96 lanes. Rare events, such as mutations, create new bands that stand out above the background pattern. Plants with bands indicative of mutations of interest were evaluated by TILLING individual members of a pool mixed with wild type DNA and then sequencing individual PCR products. Plants carrying mutations confirmed by sequencing were grown up as described above (e.g., the M2 plant could be backcrossed or outcrossed twice in order to eliminate background mutations and self-pollinated in order to create a plant that was homozygous for the mutation) or crossed to another plant containing SBEII mutations in a different homoeolog.

Plants that were identified with severe mutations in SB Ella of the A, B, or D genome were crossed with other plants that contained severe mutations in SBEIIa in other genomes. Severe mutations included those mutations that were predicted to have a deleterious effect on protein function by their SIFT and PSSM, as well as those mutations that resulted in the introduction of a stop codon (truncation mutation) or a mutation at a splice junction. Table 8 shows examples of crosses that were made.

With regard to Tables 8-12, nucleic acid designations of the mutations in SBEIIa of the A genome correspond to the position in the reference sequence SEQ ID NO: 1. Amino acid designations of the SB Ella polypeptide of the A genome correspond to the amino acid position of reference sequence SEQ ID NO: 2. Nucleic acid designations of the mutations in SBEIIa of the B genome correspond to the position in the reference sequence SEQ ID NO: 3. Amino acid designations of the SB Ella polypeptide of the B genome correspond to the amino acid position of reference sequence SEQ ID NO: 4. Nucleic acid designations of the mutations in SBEIIa of the D genome correspond to the position in the reference sequence SEQ ID NO: 5. Amino acid designations of the SBEIIa polypeptide of the A genome correspond to the amino acid position of reference sequence SEQ ID NO: 6. Nucleic acid designations of the mutations in SBEIIb of the A genome correspond to the position in the reference sequence SEQ ID NO: 7. Amino acid designations of the SBEIIb polypeptide of the A genome correspond to the amino acid position of reference sequence SEQ ID NO: 8. Nucleic acid designations of the mutations in SBEIIb of the B genome correspond to the position in the reference sequence SEQ ID NO: 9. Amino acid designations of the SBEIIb polypeptide of the B genome correspond to the amino acid position of reference sequence SEQ ID NO: 10. Nucleic acid designations of the mutations in SBEIIb of the D genome correspond to the position in the reference sequence SEQ ID NO: 11. Amino acid designations of the SBEIIb polypeptide of the A genome correspond to the amino acid position of reference sequence SEQ ID NO: 12.

TABLE 8
Examples of wheat plants identified which had a mutation in SBEIIa
that was predicted to be severe and the crosses that were made
to plants with severe SBEIIa mutations in a different genome.

| | | | Nucleotide | A.A. |
|---|---|---|---|---|
| Cross | Variety | Gene | Mutation | Mutation |
| 1 | Express | SBEIIaA | G5267A | W436* |
| | Express | SBEIIaB | G5038A | W436* |
| | Express | SBEIIaD | G6305A | W432* |
| 2 | Express | SBEIIaA | G5267A | W436* |
| | Express | SBEIIaB | G5069A | W446* |
| | Express | SBEIIaD | G6335A | W442* |
| 3 | Express | SBEIIaA | G5267A | W436* |
| | Express | SBEIIaB | G5193A | W458* |
| | Express | SBEIIaD | G6305A | W432* |
| 4 | Kronos | SBEIIaA | G5267A | W436* |
| | Kronos | SBEIIaB | G5073A | Splice |
| | | | | Junction |
| 5 | Kronos | SBEIIaA | G5267A | W436* |
| | Kronos | SBEIIaB | G5219A | G467E |
| 6 | Kronos | SBEIIaA | G5267A | W436* |
| | Kronos | SBEIIaB | G5033A | W434* |

Additionally, Express wheat plants identified as containing mutations in SBEIIa were rescreened for mutations in SBEIIb of the same genome using homoeologue specific primers. Plants with mutations in both SBEIIa and SBEIIb of each genome were sequenced and the plants containing severe mutations in both linked genes of the same genome were grown up and self-pollinated to obtain homozygous lines and confirm linkage of the mutations in SBEIIa and SBEIIb. Plants with mutations in both SBEIIa and SBEIIb in the same genome were crossed to plants with linked SBEII mutations in other genomes to obtain wheat lines with linked mutations in all three genomes.

TABLE 9: Examples of twelve Express wheat plants identified which had severe mutations in both SB Ella and SBEIIb of the same genome (i.e., linked mutations) are shown in Table 9. The SB Ella and SBEIIb genes are located close together on the chromosome and mutation segregation studies showed that these mutations were linked and were not inherited independently. It would be obvious to one skilled in the art that an alternative approach to identify linked mutations in both genes would be to first identify plants with mutations in their SBEIIb genomes and then rescreen these individuals for mutations in their SBEIIa genomes. It would also be obvious to one skilled in the art that an alternative approach to obtain linked mutations in both genes would be to identify plants in which recombination has occurred between mutations in SBEIIa and SBEIIb.

TABLE 9
Wheat plants with mutations in both SBEIIa and
SBEIIb of the same genome

| | | Nu- | A.A. | | Nu- | |
|---|---|---|---|---|---|---|
| | | cleotide | Mu- | | cleotide | A.A. |
| Plant | Gene | Mutation | tation | Gene | Mutation | Mutation |
| 1 | SBEIIaA | C5804T | P519S | SBEIIbA | C2617T | P336L |
| 2 | SBEIIaA | G5463A | G472E | SBEIIbA | G2282A | W285* |
| 3 | SBEIIaA | G5463A | G472E | SBEIIbA | G2282A | W285* |
| 4 | SBEIIaA | G5463A | G472E | SBEIIbA | G2282A | W285* |
| 5 | SBEIIaA | G2605A | G264D | SBEIIbA | G1356A | E216K |
| 6 | SBEIIaA | C5757T | A503V | SBEIIbA | G278A | W59* |
| 7 | SBEIIaD | G6306A | D433N | SBEIIbD | C4573T | R325W |
| 8 | SBEIIaD | G5156A | G374E | SBEIIbD | C4246T | P275L |
| 9 | SBEIIaD | G5156A | G374E | SBEIIbD | C4246T | P275L |
| 10 | SBEIIaD | C3743T | S266F | SBEIIbD | G4290A | V290M |
| 11 | SBEIIaB | G5219A | G467E | SBEIIbB | C3232T | R325W |
| 12 | SBEIIaB | G2386A | G233D | SBEIIbB | C2786T | P263L |

Plants that were homozygous for severe linked mutations (SBEIIa and SBEIIIb) in each genome were crossed with plants containing severe linked mutations in other genomes to create plants that had linked SBEIIa and SBEIIb mutations in all three genomes. Multiple combinations of mutations within genomes were produced during the crossing.

TABLE 10
Examples of wheat plants identified that had a severe mutation in
SBEIIa and SBEIIb of each genome and crosses to achieve plants
with mutations in both SBEIIa and SBEIIb of all three genomes.

| | | Nu- | A.A. | | Nu- | |
|---|---|---|---|---|---|---|
| | | cleotide | Mu- | | cleotide | A.A. |
| Cross | Gene | Mutation | tation | Gene | Mutation | Mutation |
| 1 | SBEIIaA | G2605A | G264D | SBEIIbA | G1356A | E216K |
| | SBEIIaB | G2386A | G233D | SBEIIbB | C2786T | P263L |
| | SBEIIaD | G6306A | D433N | SBEIIbD | C4573T | R325W |
| 2 | SBEIIaA | G2605A | G264D | SBEIIbA | G1356A | E216K |
| | SBEIIaB | G2386A | G233D | SBEIIbB | C2786T | P263L |
| | SBEIIaD | G5156A | G374E | SBEIIbD | C4246T | P275L |
| 3 | SBEIIaA | G2605A | G264D | SBEIIbA | G1356A | E216K |
| | SBEIIaB | G2386A | G233D | SBEIIbB | C2786T | P263L |
| | SBEIIaD | C3743T | S266F | SBEIIbD | G4290A | V290M |
| 4 | SBEIIaA | C5804T | P519S | SBEIIbA | C2617T | P336L |
| | SBEIIaB | G2386A | G233D | SBEIIbB | C2786T | P263L |
| | SBEIIaD | G6306A | D433N | SBEIIbD | C4573T | R325W |
| 5 | SBEIIaA | C5804T | P519S | SBEIIbA | C2617T | P336L |
| | SBEIIaB | G2386A | G233D | SBEIIbB | C2786T | P263L |
| | SBEIIaD | G5156A | G374E | SBEIIbD | C4246T | P275L |
| 6 | SBEIIaA | C5804T | P519S | SBEIIbA | C2617T | P336L |
| | SBEIIaB | G2386A | G233D | SBEIIbB | C2786T | P263L |
| | SBEIIaD | C3743T | S266F | SBEIIbD | G4290A | V290M |
| 7 | SBEIIaA | G5463A | G472E | SBEIIbA | G2282A | W285* |
| | SBEIIaB | G2386A | G233D | SBEIIbB | C2786T | P263L |
| | SBEIIaD | G6306A | D433N | SBEIIbD | C4573T | R325W |
| 8 | SBEIIaA | G5463A | G472E | SBEIIbA | G2282A | W285* |
| | SBEIIaB | G2386A | G233D | SBEIIbB | C2786T | P263L |
| | SBEIIaD | G5156A | G374E | SBEIIbD | C4246T | P275L |
| 9 | SBEIIaA | G5463A | G472E | SBEIIbA | G2282A | W285* |
| | SBEIIaB | G2386A | G233D | SBEIIbB | C2786T | P263L |
| | SBEIIaD | C3743T | S266F | SBEIIbD | G4290A | V290M |
| 10 | SBEIIaA | C5757T | A503V | SBEIIbA | G278A | W59* |
| | SBEIIaB | G2386A | G233D | SBEIIbB | C2786T | P263L |
| | SBEIIaD | G6306A | D433N | SBEIIbD | C4573T | R325W |
| 11 | SBEIIaA | C5757T | A503V | SBEIIbA | G278A | W59* |
| | SBEIIaB | G2386A | G233D | SBEIIbB | C2786T | P263L |
| | SBEIIaD | G5156A | G374E | SBEIIbD | C4246T | P275L |
| 12 | SBEIIaA | C5757T | A503V | SBEIIbA | G278A | W59* |
| | SBEIIaB | G2386A | G233D | SBEIIbB | C2786T | P263L |
| | SBEIIaD | C3743T | S266F | SBEIIbD | G4290A | V290M |
| 13 | SBEIIaA | G2605A | G264D | SBEIIbA | G1356A | E216K |
| | SBEIIaB | G5219A | G467E | SBEIIbB | C3232T | R325W |
| | SBEIIaD | G6306A | D433N | SBEIIbD | C4573T | R325W |
| 14 | SBEIIaA | G2605A | G264D | SBEIIbA | G1356A | E216K |
| | SBEIIaB | G5219A | G374E | SBEIIbB | C3232T | R325W |
| | SBEIIaD | G5156A | G467E | SBEIIbD | C4246T | P275L |
| 15 | SBEIIaA | G2605A | G264D | SBEIIbA | G1356A | E216K |
| | SBEIIaB | G5219A | G467E | SBEIIbB | C3232T | R325W |
| | SBEIIaD | C3743T | S266F | SBEIIbD | G4290A | V290M |
| 16 | SBEIIaA | C5804T | P519S | SBEIIbA | C2617T | P336L |
| | SBEIIaB | G5219A | G467E | SBEIIbB | C3232T | R325W |
| | SBEIIaD | G6306A | D433N | SBEIIbD | C4573T | R325W |
| 17 | SBEIIaA | C5804T | P519S | SBEIIbA | C2617T | P336L |
| | SBEIIaB | G5219A | G467E | SBEIIbB | C3232T | R325W |
| | SBEIIaD | G5156A | G374E | SBEIIbD | C4246T | P275L |
| 18 | SBEIIaA | C5804T | P519S | SBEIIbA | C2617T | P336L |
| | SBEIIaB | G5219A | G467E | SBEIIbB | C3232T | R325W |
| | SBEIIaD | C3743T | S266F | SBEIIbD | G4290A | V290M |
| 19 | SBEIIaA | G5463A | G472E | SBEIIbA | G2282A | W285* |
| | SBEIIaB | G5219A | G467E | SBEIIbB | C3232T | R325W |
| | SBEIIaD | G6306A | D433N | SBEIIbD | C4573T | R325W |
| 20 | SBEIIaA | G5463A | G472E | SBEIIbA | G2282A | W285* |
| | SBEIIaB | G5219A | G467E | SBEIIbB | C3232T | R325W |
| | SBEIIaD | G5156A | G374E | SBEIIbD | C4246T | P275L |
| 21 | SBEIIaA | G5463A | G472E | SBEIIbA | G2282A | W285* |
| | SBEIIaB | G5219A | G467E | SBEIIbB | C3232T | R325W |
| | SBEIIaD | C3743T | S266F | SBEIIbD | G4290A | V290M |
| 22 | SBEIIaA | C5757T | A503V | SBEIIbA | G278A | W59* |
| | SBEIIaB | G5219A | G467E | SBEIIbB | C3232T | R325W |
| | SBEIIaD | G6306A | D433N | SBEIIbD | C4573T | R325W |
| 23 | SBEIIaA | C5757T | A503V | SBEIIbA | G278A | W59* |
| | SBEIIaB | G5219A | G467E | SBEIIbB | C3232T | R325W |
| | SBEIIaD | G5156A | G374E | SBEIIbD | C4246T | P275L |
| 24 | SBEIIaA | C5757T | A503V | SBEIIbA | G278A | W59* |
| | SBEIIaB | G5219A | G467E | SBEIIbB | C3232T | R325W |
| | SBEIIaD | C3743T | S266F | SBEIIbD | G4290A | V290M |

TABLE 11
Three examples of wheat plants with other combinations of mutations of
SBEIIa and SBEIIb of multiple genomes.

| | | Nucleotide | A.A. | | Nucleotide | A.A. |
|---|---|---|---|---|---|---|
| Type | Gene | Mutation | Mutation | Gene | Mutation | Mutation |
| SBEIIa Only | SBEIIaA | G5267A | W436* | SBEIIbB | C2786T | P263L |
| LinkedSBEIIa & IIb | SBEIIaB | G2386A | G233D | SBEIIbD | C4573T | R325W |
| LinkedSBEIIa & IIb | SBEIIaD | G6306A | D433N | | | |
| LinkedSBEIIa & IIb | SBEIIaA | G2605A | G264D | SBEIIbA | G1668A | E216K |
| SBEIIa Only | SBEIIaB | G5038A | W436* | | | |
| LinkedSBEIIa & IIb | SBEIIaD | G6306A | D433N | SBEIIbD | C4573T | R325W |
| LinkedSBEIIa & IIb | SBEIIaA | G2605A | G264D | SBEIIbA | G1668A | E216K |
| LinkedSBEIIa & IIb | SBEIIaB | G2386A | G233D | SBEIIbB | C2786T | P263L |
| SBEIIa Only | SBEIIaD | G6305A | W432* | | | |

TABLE 12
Additional examples of wheat plants with other combinations of
mutations of SBEIIa and SBEIIb of multiple genomes.

| | | | Nucleotide | A.A. | | Nucleotide | |
|---|---|---|---|---|---|---|---|
| Combo | Type | Gene | Mutation | Mutation | Gene | Mutation | A.A. Mutation |
| 1 | LinkedSBEIIa & IIb | SBEIIaA | G5267A | W436* | SBEIIbA | G2282A | W285* |
| | LinkedSBEIIa & IIb | SBEIIaB | G5038A | W436* | SBEIIbB | G1916A | S208N |
| | LinkedSBEIIa & IIb | SBEIIaD | G6305A | W432* | SBEIIbD | G3599A | W233* |
| 2 | SBEIIa Only | SBEIIaA | G5267A | W436* | SBEIIbA | | |
| | LinkedSBEIIa & IIb | SBEIIaB | G5038A | W436* | SBEIIbB | G1916A | S208N |
| | LinkedSBEIIa & IIb | SBEIIaD | G6305A | W432* | SBEIIbD | G3599A | W233* |
| 3 | LinkedSBEIIa & IIb | SBEIIaA | G5267A | W436* | SBEIIbA | G2282A | W285* |
| | SBEIIa Only | SBEIIaB | G5038A | W436* | SBEIIbB | | |
| | LinkedSBEIIa & IIb | SBEIIaD | G6305A | W432* | SBEIIbD | G3599A | W233* |
| 4 | LinkedSBEIIa & IIb | SBEIIaA | G5267A | W436* | SBEIIbA | G2282A | W285* |
| | LinkedSBEIIa & IIb | SBEIIaB | G5038A | W436* | SBEIIbB | G1916A | S208N |
| | SBEIIa Only | SBEIIaD | G6305A | W432* | SBEIIbD | | |
| 5 | SBEIIa Only | SBEIIaA | G5267A | W436* | SBEIIbA | | |
| | SBEIIa Only | SBEIIaB | G5038A | W436* | SBEIIbB | | |
| | LinkedSBEIIa & IIb | SBEIIaD | G6305A | W432* | SBEIIbD | G3599A | W233* |
| 6 | LinkedSBEIIa & IIb | SBEIIaA | G5267A | W436* | SBEIIbA | G2282A | W285* |
| | SBEIIa Only | SBEIIaB | G5038A | W436* | SBEIIbB | | |
| | SBEIIa Only | SBEIIaD | G6305A | W432* | SBEIIbD | | |
| 7 | SBEIIa Only | SBEIIaA | G5267A | W436* | SBEIIbA | | |
| | LinkedSBEIIa & IIb | SBEIIaB | G5038A | W436* | SBEIIbB | G1916A | S208N |
| | SBEIIa Only | SBEIIaD | G6305A | W432* | SBEIIbD | | |
| 8 | LinkedSBEIIa & IIb | SBEIIaA | G5267A | W436* | SBEIIbA | G2156A | Splice Junction |
| | LinkedSBEIIa & IIb | SBEIIaB | G5038A | W436* | SBEIIbB | C3232T | R325W |
| | LinkedSBEIIa & IIb | SBEIIaD | G6305A | W432* | SBEIIbD | C4573T | R325W |
| 9 | SBEIIa Only | SBEIIaA | G5267A | W436* | SBEIIbA | | |
| | LinkedSBEIIa & IIb | SBEIIaB | G5038A | W436* | SBEIIbB | C3232T | R325W |
| | LinkedSBEIIa & IIb | SBEIIaD | G6305A | W432* | SBEIIbD | C4573T | R325W |
| 10 | LinkedSBEIIa & IIb | SBEIIaA | G5267A | W436* | SBEIIbA | G2156A | Splice Junction |
| | SBEIIa Only | SBEIIaB | G5038A | W436* | SBEIIbB | | |
| | LinkedSBEIIa & IIb | SBEIIaD | G6305A | W432* | SBEIIbD | C4573T | R325W |
| 11 | LinkedSBEIIa & IIb | SBEIIaA | G5267A | W436* | SBEIIbA | G2156A | Splice Junction |
| | LinkedSBEIIa & IIb | SBEIIaB | G5038A | W436* | SBEIIbB | C3232T | R325W |
| | SBEIIa Only | SBEIIaD | G6305A | W432* | SBEIIbD | | |
| 12 | SBEIIa Only | SBEIIaA | G5267A | W436* | SBEIIbA | | |
| | SBEIIa Only | SBEIIaB | G5038A | W436* | SBEIIbB | | |
| | LinkedSBEIIa & IIb | SBEIIaD | G6305A | W432* | SBEIIbD | C4573T | R325W |
| 13 | LinkedSBEIIa & IIb | SBEIIaA | G5267A | W436* | SBEIIbA | G2156A | Splice Junction |
| | SBEIIa Only | SBEIIaB | G5038A | W436* | SBEIIbB | | |
| | SBEIIa Only | SBEIIaD | G6305A | W432* | SBEIIbD | | |
| 14 | SBEIIa Only | SBEIIaA | G5267A | W436* | SBEIIbA | | |
| | LinkedSBEIIa & IIb | SBEIIaB | G5038A | W436* | SBEIIbB | C3232T | R325W |
| | SBEIIa Only | SBEIIaD | G6305A | W432* | SBEIIbD | | |
| 15 | LinkedSBEIIa & IIb | SBEIIaA | G5267A | W436* | SBEIIbA | G2282A | W285* |
| | LinkedSBEIIa & IIb | SBEIIaB | G5038A | W436* | SBEIIbB | C3232T | R325W |
| | LinkedSBEIIa & IIb | SBEIIaD | G6305A | W432* | SBEIIbD | C4573T | R325W |
| 16 | LinkedSBEIIa & IIb | SBEIIaA | G5267A | W436* | SBEIIbA | G2282A | W285* |
| | LinkedSBEIIa & IIb | SBEIIaB | G5038A | W436* | SBEIIbB | C3232T | R325W |
| | LinkedSBEIIa & IIb | SBEIIaD | G6305A | W432* | SBEIIbD | G3599A | W233* |
| 17 | LinkedSBEIIa & IIb | SBEIIaA | G5267A | W436* | SBEIIbA | G2282A | W285* |
| | LinkedSBEIIa & IIb | SBEIIaB | G5038A | W436* | SBEIIbB | G1916A | S208N |
| | LinkedSBEIIa & IIb | SBEIIaD | G6305A | W432* | SBEIIbD | C4573T | R325W |
| 18 | SBEIIa Only | SBEIIaA | G5267A | W436* | SBEIIbA | | |
| | LinkedSBEIIa & IIb | SBEIIaB | G5038A | W436* | SBEIIbB | C3232T | R325W |
| | LinkedSBEIIa & IIb | SBEIIaD | G6305A | W432* | SBEIIbD | G3599A | W233* |
| 19 | SBEIIa Only | SBEIIaA | G5267A | W436* | SBEIIbA | | |
| | LinkedSBEIIa & IIb | SBEIIaB | G5038A | W436* | SBEIIbB | G1916A | S208N |
| | LinkedSBEIIa & IIb | SBEIIaD | G6305A | W432* | SBEIIbD | G3599A | W233* |
| 20 | SBEIIa Only | SBEIIaA | G5267A | W436* | SBEIIbA | | |
| | LinkedSBEIIa & IIb | SBEIIaB | G5038A | W436* | SBEIIbB | G1916A | S208N |
| | LinkedSBEIIa & IIb | SBEIIaD | G6305A | W432* | SBEIIbD | C4573T | R325W |
| 21 | LinkedSBEIIa & IIb | SBEIIaA | G5267A | W436* | SBEIIbA | G2282A | W285* |
| | SBEIIa Only | SBEIIaB | G5038A | W436* | SBEIIbB | | |
| | LinkedSBEIIa & IIb | SBEIIaD | G6305A | W432* | SBEIIbD | C4573T | R325W |
| 22 | LinkedSBEIIa & IIb | SBEIIaA | G5267A | W436* | SBEIIbA | G2156A | Splice Junction |
| | SBEIIa Only | SBEIIaB | G5038A | W436* | SBEIIbB | | |
| | LinkedSBEIIa & IIb | SBEIIaD | G6305A | W432* | SBEIIbD | G3599A | W233* |
| 23 | LinkedSBEIIa & IIb | SBEIIaA | G5267A | W436* | SBEIIbA | G2282A | W285* |
| | LinkedSBEIIa & IIb | SBEIIaB | G5038A | W436* | SBEIIbB | C3232T | R325W |
| | SBEIIa Only | SBEIIaD | G6305A | W432* | SBEIIbD | | |
| 24 | LinkedSBEIIa & IIb | SBEIIaA | G5267A | W436* | SBEIIbA | G2156A | Splice Junction |
| | LinkedSBEIIa & IIb | SBEIIaB | G5038A | W436* | SBEIIbB | G1916A | S208N |
| | SBEIIa Only | SBEIIaD | G6305A | W432* | SBEIIbD | | |

Mutations in SBEIIa increase amylose content and resistant starch levels in wheat seeds from (1) double homozygous Kronos wheat plants with a stop mutation in SBEIIaA (G 5267A/W436*) and a splice junction mutation in SBEIIaB (G5073A/splice junction), and (2) double homozygous Kronos wheat plants with a stop mutation in SBEIIIaA (G5267A/W436*) and a missense mutation in SBEIIIaB (G5219A/G467E) were evaluated for amylose content using the K-AMYL kit from Megazyme (Ireland) and controls of known amylose amounts. The amylose content of whole seed milled starch was an average of 40-49% for the double homozygous mutant seeds compared to seeds from their wild type sibling controls whose amylose content was 20-25%.

Seeds from (1) triple homozygous Express wheat plants with a stop mutation in SBEIIaA (G5267A/W436*), SBEIIaB (G5038A/W436*), and SBEIIaD (G6305A/W432*), and (2) triple homozygous Express wheat plants with a stop mutation in SBEIIaA (G5267A/W436*), SBEIIaB (G5069A/W 446*), and SBEIIaD (G 6335A/W 442*) were evaluated for amylose content using the K-AMYL kit from Megazyme (Ireland) and a controls of known amylose amounts. The amylose content of whole seed milled starch was 50-60% for the triple homozygous mutant seeds compared to seeds from their wild type sibling controls whose amylose content was 20-25%.

Flour milled from the triple homozygous mutant seed had 12-15% resistant starch content compared to flour from the wild type sibling controls, which had approximately 1% resistant starch. Bread made from the triple homozygous mutant flour had increased resistant starch levels of 6% compared to bread made from flour of wild type sibling and parental controls, which had less than 1% resistant starch. Bread made from a 50:50 blend with standard wheat flour had increased resistant starch levels of 4% compared to bread made from a 50:50 blend with sibling control flour that had resistant starch levels less than 1%.

Seeds from (1) quadruple homozygous Express wheat plants with a linked mutation in SBEIIaA (G5463A/G472E)- and SBEIIbA (G2282A/W285*), combined with a stop mutation in SBEIIaB (G5038A/W 436*), and SBEIIaD (G 6305A/W432) was evaluated for amylose content using the K-AMYL kit from Megazyme (Ireland) and controls of known amylose amounts. The amylose content of whole seed milled starch was 58% for the quadruple homozygous mutant seeds compared to seeds from their wild type sibling controls whose amylose content was 20-25%.

Seeds from (2) quadruple homozygous Express wheat plants with a stop mutation in SBEIIaA (G5267A/W436*), combined with a stop mutation in SBEIIaB (G5038A/W436*), and a linked mutation in SBEIIaD (G6306A/D433N)- and SBEIIbD (C4573T/R325W) was evaluated for amylose content using the K-AMYL kit from Megazyme (Ireland) and controls of known amylose amounts. The amylose content of whole seed milled starch was 38% for the quadruple homozygous mutant seeds compared to seeds from their wild type sibling controls whose amylose content was 23%.

Seeds from (3) quadruple homozygous Express wheat plants with a stop mutation in SBEIIaA (G5267A/W436*), combined with a linked mutation in SBEIIaB (G5219A/G467E)- and SBEIIbB (C3232T/R325W), and a stop mutation in SBEIIaD (G6305A/W432*) were evaluated for amylose content using the K-AMYL kit from Megazyme (Ireland) and controls of known amylose amounts. The amylose content of whole seed milled starch was 38% for the quadruple homozygous mutant seeds compared to seeds from their wild type sibling controls whose amylose content was 24%.

Seeds from a sextuple homozygous Express wheat plants with linked mutations in SBEIIaA (G5463A/G472E) and SBEIIbA (G2282A/W285*), combined with linked mutations in SBEIIaB (G5219A/G467E) and SBEIIbB (C3232T/R325W), and linked mutations in SBEIIaD (G6306A/D433N) and SBEIIbD (C4573T/R 325W) were evaluated for amylose content using the K-AMYL kit from Megazyme (Ireland) and controls of known amylose amounts. The amylose content of whole seed milled starch was 25-30% for the sextuple homozygous mutant seeds compared to seeds from their wild type sibling controls whose amylose content was 16%.

The above examples are provided to illustrate the invention but not limit its scope. Other variants of the invention will be readily apparent to one of ordinary skill in the art and are encompassed by the appended claims and all their equivalents. The examples above used TILLING technology to create and identify mutations in one or more SBEII genes of wheat that increase amylose levels in wheat seeds, but one of ordinary skill in the art would understand that other methods such as targeted mutagenesis (also known as site-directed mutagenesis, site-specific mutagenesis or oligonucleotide-directed mutagenesis) could be used to create the useful mutations of the present invention in one or more SBEII loci of wheat (see for example Zhang et al., PNAS 107(26):12028-12033, 2010; Saika et al., Plant Physiology 156:1269-1277, 2011). All publications, patents, and patent applications cited herein are hereby incorporated by reference.

1.-18. (canceled)

19. An SBEIIa polynucleotide from a wheat plant of the species Triticum turgidum L. ssp durum encoding an SBEIIa polypeptide having a mutation, wherein the mutation is W436* at a position corresponding to W436 of SEQ ID NO:2, a splice junction mutation at a position corresponding to G5073A of SEQ ID NO:3, or G467E at a position corresponding to G467 of SEQ ID NO:4, wherein * indicates a stop codon.

20. The SBEIIa polynucleotide of claim 19, wherein the mutation is W436* at a position corresponding to W436 of SEQ ID NO:2.

21. The SBEIIa polynucleotide of claim 19, wherein the mutation is a splice junction mutation at a position corresponding to G5073A of SEQ ID NO:3.

22. The SBEIIa polynucleotide of claim 19, wherein the mutation is G467E at a position corresponding to G467 of SEQ ID NO:4.

23. A wheat plant of the species Triticum turgidum L. ssp durum comprising one or more polynucleotides of claim 19.

24. A wheat plant of the species Triticum turgidum L. ssp durum comprising one or more polynucleotides of claim 20.

25. A wheat plant of the species Triticum turgidum L. ssp durum comprising one or more polynucleotides of claim 21.

26. A wheat plant of the species Triticum turgidum L. ssp durum comprising one or more polynucleotides of claim 22.

27. The wheat plant of the species Triticum turgidum L. ssp durum of claim 23 comprising one or more mutations of each SBEIIa gene of the A and B genomes selected from:
(i) the one or more mutations in the SBEIIa gene of the A genome is W436* at a position corresponding to W436 of SEQ ID NO:2 and the one or more mutations in the SBEIIa gene of the B genome is a splice junction mutation at a position corresponding to G5073A of SEQ ID NO:3; and
(ii) the one or more mutations in the SBEIIa gene of the A genome is W436* at a position corresponding to W436 of SEQ ID NO:2 and the one or more mutations in the SBEIIa gene of the B genome is G467E at a position corresponding to G467 of SEQ ID NO:4.

28. A wheat grain from the plant of claim 27.

29. A flour comprising a component of the wheat grain of claim 28.

30. A food product comprising a component of the wheat grain of claim 28.

31. A food product comprising the wheat flour of claim 29.

32. The food product of claim 30, wherein the food product is a pasta.

33. A beverage product comprising the wheat flour of claim 29.

34. A plant part or progeny thereof from the wheat plant of claim 27, wherein the plant part or progeny comprises the one or more mutations of each SBEIIa gene of the A and B genomes.


SEQUENCE LISTING (58 sequences)

SEQ ID NO: 1 - DNA, 6114 bp, Triticum aestivum
  source 1..6114: mol_type = unassigned DNA
caattaatatcgtccatcactcgggttccgcgctgcatttcggccggcgggttgagtgagatctgggccactgaccgactcactcgctcgctgcgcggggatggcgacgtttgcggtgtccggcgcgaccctcggtgtggcgcggcccgccggcgccggcggcggactgctgccgcgatccggctcggagcggaggggcggggtggacctgccgtcgctgctcctcaggaagaaggactcctctcgtacgcctcgctcgctcgctccaatctcccgtccatttttgccccccttctctctccctatctgcgcgcgcatggcctgttcgatgctgttccccagttgatctccatcaacgagagagatagctggattaggcgatcgcctgcgtcagtgtcacccaggccctggtgttatcacggctttgatcatctcctcccattctgatattttctcactctttcttctgttcttgctgtaactgcaagttgtagcattgtctcactattgtagtcatccttgcattgcaggcgccgtcctgagccgcgcggcctctccagggaaggtcctggtgcctgacggtgagagcgacgacttggcaagtccggcgcaacctgaagaattacaggtacacaccatcgtgccgggaaatcttcatacaatcgttattcacttaccaaatgccggatgaaaccaagccgcggaggcgtcaggttttgagcttcttctatcagcattgtgcagtactgcactgccttgtgcattttgttagccgtggccccgtgctggctcttgggccactgaaaactcagatggatgtgcattctagcaagaacttcacgaaataatgcactgtttgtggtttcgttagtctgctctacaattgctattttcgtgctgtagatacctgaagacatcgaggagcaaacggctgaagtaaacatgacaggggggactgcagaaaaacttgaatcttcagaaccgactcaaggcattgtggaaacaatcactgatggtgtaaccaaaggagttaaggaactagtcgtgggggagaaaccgcgagttgtcccaaaaccaggagatgggcagaaaatatacgagattgacccaacgctgaaagattttcggagccatcttgactaccggtaatgcctacccgctactttcgctcattttgaattaaggtcctttcgtcatgcaaatttggggaacatcaaagagacaaagactagggaccactatttcttacagttcccctcatggtctgagaatatgctgggacgtagatgtataattgatggctacaatttgctcataattacgatacaaataactgtctctgatcattgcaattacagagtggcaaactgattaaaatgtgatagatgggttatagattttactttgctaattcctctaccaaattcctggggaaaaaaatctaccagttgggcaacttagtttcttatctttgttgcctctttgttttggggaaaacacactgctaaatttgaatgattttgggtatgcctccgtggattcaacagatacagcgaatacaggagaattcgtgctgctattgaccaacatgaaggtggattggaagcattttctcgtggttatgaaaagcttggatttacccgcaggtaaatttaaagcttcagtattatgaagcgcctccactagtctacttgcatatcttacaagaaaatttataattcctgttttcgcctctcttttttccagtgctgaaggtattgtctagttgcatatcttataagaaaatttatgttcctgttttcccctattttccagtgctgaaggtatcacttaccgagaatgggctcctggagcgcatgtacgtcttttaagtcttaacagacaccttccaattcattgttaatggtcacactattcaccaactagcttactggacttacaacttagcttactgaatactgaccagttgctctaaatttatgatctggcttttgcatcctattacagtctgcagcattagtaggtgacttcaacaattggaatccgaatgcagatactatgaccagagtatgtctacagcttggcaatcttccacctttgcttcataactactgatacatctatttgtatttattttgctgtttgcacattccttaaagttgagcctcaactatatcatatcaaaatggtataatttgtcagtgtcttaagcttcagcctaaagattctactcaaattggtccatctttttgagattgaaaatgagtatattaaggatggatgaataggtgcaacactcccattctttggtagaaccttctgcattatgtgtgttttttcatctacaatgagcatatttccatgctatcagtgaaggtttgctcctattgatgccgatatttgatatgatcttttcaggatgattatggtgtttgggagattttcctccctaacaatgctgatggatccccagctattcctcatggctcacgtgtaaaggtaagctggccaattatttagttgaggatgtagcattttcgaactctgcccactaagggtccctttgcctttctgttttctagatacggatggatactccatctggtgtgaaggattcaatttctgcttggatcaagttctctgtgcaggctccaggtgaaataccattcaatggcatatattatgatccacctgaagaggtaagtatcgatctccattacattattaaatgaaatttccagtgttacggttttttaatacccatttcgtgtctcactgacatgtgagtcaagacaatactttagaatttggaagtgacatatgcattaattcaccttctaagggctaaggggcaagcaaccatggtgatgtttgtatgcttgtgtgtgacttaagatcttatagctcttttatgtgttctctgttggttaggatattccattttgaccttttgtgaccatttactaaggatatttacatgcaaatgcaggagaagtatgtcttccaacatcctcaacctaaacgaccagagtcactgaggatttatgaatcacacattggaatgagcagcccagtatgtcaataagttatttcacctgtttctggtctgatggtctattctatggattttttagttctgttatgtattgttaacatataacatggtgcattcacgtgacaacctcgattttattttctaatgttattgcaatagctcggtataatgtaaccatgttactagcttaagatggttagggtttcccacttaggatgcatgaaatatcgcattggagcatctccagcaagccatttttttgacggttaacagcaggagctctgcttttcattataggagagggaaatgctgtacagactgaagtcagtcagagcaaagtaacttagaatcatttatgggccaccctgcacagggcagaaggcaggcaggaacgatcctctacagccgtcggattgcctccatcagaggaatcctggccgttaatcatgctctggcccagtggtcagaatgcatcaaccagactgaggtgcttgcctccttattggtaaaggatgcagcggtacgagcctattgaacagatcctgttcaagtaaggccgttctccagcaagccatttcctagcttattaatgagagagagagagagaggggggggggtctgtattctgcgagcaattcaaaaacttccattgttctgaggtgtacgcattgtagggatctcccattatgaagaggatatagttaattctttgtaacctacttggaaacttgagtcttgcggcatcgctaatatattctatcatcacaatacttagaggatgcatctgaatattttagtgggatcttgcacaggaaccgaagataaattcatatgctaattttagggatgaggtgctgccaagaattaaaaggcttggatacaatgcagtgcagataatggcaatccaggagcattcatactatgcgagctttgggtattcacacaatccatttttttctgttcttttttctgtatgcgcctcttcacccatttggagctattacatcctaatgcttcgtgcacatagaatatttggatataattctttagtagacatatagtacaacaacagttggtatttctgacttgtatgaccattttattgttgttggcttgttccaggtaccatgttactaatttttttgcaccaagtagccgttttggaactccagaggacttaaaatccctgatcgatagagcacatgagcttggtttgcttgttcttatggatattgttcataggtaagtagtccaattaattttagctgctttactgtttatctggtattctaaatggcagggccgtatcgacgagtatttttccattctatataattgtgctacatgacttcttttttctcagatgtattaaaccagttggacatcaaatgtatttggtacatctagtaaactgacagtttcaaagaacatcgttttgtaatggcaacatgatttgatgccatagatgtggactgagaagttcagatgctatcaagaaaattaatcaactggccatgtactcgtggcactacatagagtttgcaagttggaaaactgacagcaatacctcactgataagtagctaggccccacttgccagcttcatattagatgttacttccctgttgaactcatttgaacatattacttaaagttcttcatttgtcctaagtcaaacttctttaagtttgaccaagtctactgaaaaatatatcaacatctacaacaccaaattggcttcattagattcacaatttttattttgtaatattagcacacctttgatgttgtagatatcagcacatttttctacagacttggtcaaatatagagaagtttgacttaggacaaatctagaacttcaatcaatttggatcagaggggatagtccatactggttgattatatccggtaacatcaaataatatagatagatgtcaacactttaacaaaaaaatcagaccttgtcaccaaatatgtatcagaccatctgtttgctttagccacttgttttcatatttatgtgtttgtacctaatctatttttacttctacttggtttggttgattttttttcagttgcattgcttcatcaatgattttgtgtaccctgcagtcattcatcaaataatacccttgacggcttgaatggtttcgatggcactgatacacattacttccacggtggtccacgtggccatcattggatgtgggattctcgtctattcaactatgggagttgggaagtatgtagctctgacttctgtcaccatatttggctaactgttcctgttaaatctgttcttacacatgtcgatattctattcttatgtaggtattgagattcttactgtcaaacgcgagatggtggcttgaagaatataagtttgatggatttcgatttgatggggtgacctccatgatgtatactcaccatggattacaagtaagtcatcaagtggtttcagtaacttttttagggcactgaaataattgctatgcatcataacatgtatcatgatcaggacttgtgctacggagtcttagatagttccctagtacgcttgtacaattttacctgatgagatcatggacgattcgaagtgattattatttatttttcttctaagtttgcttcttgttctagatgacatttactgggaactatggcgagtattttggatttgctactgatgttgacgcggtagtttacttgatgctggtcaacgatctaattcatggacttcatcctgatgctgtatccattggtgaagatgtaagtgcttacagtatttatgatttttaaccagttaagtagttttattttgggatcaggctgttactctttttgttaggggtaagatctctcttttcataacaatgctaatttataccttgtatgataatgcatcacttaggtaatttgaaaagtgcaaggccattcaagcttacgagcatattttttgatggctgtaatttatttgatagtatgcttgtttgggtttttcagtaaatgggagtgtgtgactaatgttgcattagaaatgggcaaccttgtca

SEQ ID NO: 2 - protein, 527 aa, Triticum aestivum
MATFAVSGATLGVARPAGAGGGLLPRSGSERRGGVDLPSLLLRKKDSSRAVLSRAASPGKVLVPDGESDDLASPAQPEELQIPEDIEEQTAEVNMTGGTAEKLESSEPTQGIVETITDGVTKGVKELVVGEKPRVVPKPGDGQKIYEIDPTLKDFRSHLDYRYSEYRRIRAAIDQHEGGLEAFSRGYEKLGFTRSAEGITYREWAPGAHSAALVGDFNNWNPNADTMTRDDYGVWEIFLPNNADGSPAIPHGSRVKIRMDTPSGVKDSISAWIKFSVQAPGEIPFNGIYYDPPEEEKYVFQHPQPKRPESLRIYESHIGMSSPEPKINSYANFRDEVLPRIKRLGYNAVQIMAIQEHSYYASFGYHVTNFFAPSSRFGTPEDLKSLIDRAHELGLLVLMDIVHSHSSNNTLDGLNGFDGTDTHYFHGGPRGHHWMWDSRLFNYGSWEVLRFLLSNARWWLEEYKFDGFRFDGVTSMMYTHHGLQMTFTGNYGEYFGFATDVDAVVYLMLVNDLIHGLHPDAVSIGED

SEQ ID NO: 3 - DNA, 10219 bp, Triticum aestivum
  source 1..10219: mol_type = unassigned DNA
tgagatctgggccactgaccgactcactcgctgcgcggggatggcgacgttcgcggtgtccggcgcgaccctcggtgtggcgcggcccgccagcgccggcggcggactgctgcgatccggctcggagcggaggggcggggtggacttgccgtcgctgctcctcaggaagaaggactcctctcgtacgcctcgctccctccaatctccccgtctgtttttgggcccccttctctctccctcgcctctctgcgcgcgcatggcctgttcgatgctgttccccagttgatctccatgaacgagagagatagctggattaggcgatcgcctcaggccctggtgttaccacggctttgatcatctcctcctttcatgctgatattttctcactctttcttctgttcttgctgtaactgcaagttgtagcatttttttggcgaataagttgtagcattgtctcactattgtactcatccttgcatttgcaggcgccgtcctgagccgcgcggcctctccagggaaggtcctggtgcctgacggtgagagcgacgacttggcggccactccagcgcaacccgaagaattacaggtacacaccgtcgtgccggaaaatcttcatgcacccgttattcacttaccaaatatcggatgaaccaagccgcggaggcatcaggtttcaagcttcttctatcagcattgtgcactacttcactgccttgtgcagtttgttagctgtggccccgcgctggctcttgggccactgaaaactcagatggatgtgcattctagcaagaacttcacaaaataatgcactgtttgtggtttcgttagtctgctctacaattgctatttttcgtgtgctgtagatacctgaagatatcgaggagcaaacggctgaagtgaacatgacaggggggactgcagagaaacttcaatattcagaaccgactcagggcattgtggaaacaatcactgatggtgtaaccaaaggagttaaggaactagtcgtgggggagaaaccgcgagttgtcccaaaaccaggagatgggcagaaaatatacgagattgacccaacgctgaaagattttcggagccatcttgactaccggtaatgcctacccgctaatttcgctcattttgaattaaggtcctttcatcatgcaaatttggggaacatcaaagaggcaaagactagggaccactgtttcatacagttcccctcatggtctgagaatatgctgggaagtatatgtataattgctggctacaattggctcataattgcaatacaaataactgtctccgatcattacaattacagagtggcaaactgatgaaaatgtggtggatgggttatggattttactttgctaattcctctaccaaattcctggggaaaaaatctaccagttgggcaacttagtttcttatctttgttgcctttttgttttggggaaaacacactgctaaatttgaatgattttgggtatgccttggtggattcaacagatacagcgaatacaagagaattcgtgctgctattgaccaacatgaaggtggattggaagcattttctcgtggttatgaaaagcttggatttacccgcaggtaaatttaaagctttactatgaaacgcctccactagtctaattgcatatcttgtaagaaaatttataattcctgttttcccctctcttttttccagtgctgaaggtatcatctaattgcttatcttataagaaaatttataattcctgtttccccccctctttttccagtgctgaaggtatcacttaccgagaatgggctcctggagcgcatgtacgtcttaacagacaccttctaatctattgttaatggtcactattcaccaactagcttactgaacttacaaaatagcttactgaatactgaccagttactctaaatttatgatctggcttttgcatcctgttacagtctgcagcattagtaggtgacttcaacaattggaatccaaatgcagatactatgaccagagtatgtctacagcttggcaatcttccacctttgcttcgtaactactgatacatctatttgtatttatttaactgtttgcacgttcgttaaagttgagcctcaactatatcataccaaaatggtataatttgtcagtgtcttaagcttcagcctaaagatcctactgaatttagtccatccttttgagattgaaaatgagtatattaagggtgattgaatacttgcaacactcccattttttggtagaaccttttgcattatgtgtgcttttccatccacaatgagcatatttccatgttatcagtgaaggtttgctcctattgatgccgatatttgatatgatctttcgatcttttcaggatgattatggtgtttgggagatcttcctccctaacaatgctgatggatccccagctattcctcatggctcacgtgtaaaggtaatctggccaattatttagtcgaggatgtaacattttcgaactctgcctactaagggtcccttttcctctctattttctagatacggatggatactccatctggtgtgaaggattcgatttctgcttggatcaagttctctgtgcaggctccaggtgaaataccattcaatggcatatattatgatccacctgaagaggtaagtatcaatctatgttacattattaaatggaatttccagtgttacagttttttgatacccacttcatgtctcactgacatgtgagtcaagacaatactttcgaatttggaagtgacatatgcattaattcaccttctaagggctaaggggcaaccaaccatggtgatgtgtgtatgcttgtgtgacttaagatcttatagctcttttatatgttctctgttggttaggacattccattttgaccttttgtgaccatttactaaggatattttacatgcaaatgcaggagaagtatgtcttccaacatcctcaacctaaacgaccagagtcactaaggatttatgaatcacacattggaatgagcagcccggtatgtcaataagttatttcacctgtttccggtctgatggtttattctatggattttctagttctgttatgtactgttaacataccacacggtgcattcacgtgacaacctcgattttattttctaatgtcttcatattggaaaatgcacaactttgcttcctctttgtctgatcgtttttttgtctctaagatttccattgcatttcgaggtagcgggcatgtgaaagtcgaatctgaatattttttgtcagagcacagttatattaaatgccattgttgttgcaatagcttggtataatgtagccatgttactagcttaagaaatatcgcattggagcatctccagcaagccatttcctaccttattactgagggggggggggggggggagcggggttctgtattctgcgagcgattcaaaacttccactgttctgaggtgtacgtactgtagggatctcccattatgaagaggacatagttaactttttgtaacctacttggaaacttgagtcttgatgcatcgctactatatactatcatcacaatacttagaggatgcatctgaatattttagcgtgatcttgcacaggaaccgaagataaattcatatgctaattttagggatggggtgctgccaagaattaaaaggcttggatacaatgcagtgcagataatggcaatccaggagcattcatactatgcaagctttgggtattcatacagtccatctttttctgttttttttttctgtatgtgcctcttcacccatttcgagccattacatcctaatgcttcgtgcacataaaatacttggatataattctttattagacatatagtacaacaccacttagtatttctgacttgtatgatcattttattgttgttggcttgttacaggtaccatgttactaatttttttgcaccaagtagccgttttggaactccagaggacttaaaatccttgatcgatagagcacatgagcttggtttgcttgttcttatggatattgttcataggtaatcagtccaatttaattttagttgctttactgtttatctggtattgtaaatggcagggccctatcgtcgaatatttttccaatctatataattgtgctacatgacttattttttctcagatgtattaaaccagttggatattaaatgtatttggtacatctagtaaactgacagtttcatagaattgtgttgtaatggcaacacaatttgatggcatagatgtggactgagaagttcagatgctatcagtaattaattaactggccatgtactcgtggaactacatagagtttgcaagttggaaaactgacagcaatacctcactgataagtgtccaggccacacttgccagcttcatattagatgttacttccctgttgaactcctttgaacatatcacttaaagttcttcaattgtcctaagtcaaacttctttgactttggccaagtctattgaaaaatatgtcaacatctacagcaccaaattagtttcataatttttattttgttatattagcacgttttttatgctgtagatatcagcacatttttctatagacttggtcaaatatagagaagtttgacttaggacaaatcagaacttcaagcaatttggatcagagggaatagtccatactgcttgattatattttcccaaaggagggagtgaggagcttgacttcggtatcatcaaatgatattgatagatgtcaacattttaacaaaaaatcagaccttgtcaccaaatatgcatcagaccatctgtttgcttaggcacttgctttcatatttatgtgtttgtaactaatctacttttccttctacttggtttgattgattctatttcagttgcattgcttcatcaatgattttgtgtaccctgcagtcattcgtcaaataatacccttgacggtttgaatggtttcgatggcactgatacacattacttccacggtggtccacgtggccatcattggatgtgggattctcgtctgttcaactatgggagttgggaagtatgtagctgcgacttctgtcaccatgtttggctaactgttcctgccaatctgttcttacacgtgtcaatattctattcttatacaggtattaagattcttactgtcaaacgcgagatggtggcttgaagaatataagtttgatggatttcgatttgatggggtgacctccatgatgtatactcaccatggattacaagtaagtcatcaagtggtttcagtaacttcttcagggcactgaaacaattgctatgcatcataacatgtatcatgatcagtacttatgctacggagtcttagatagttccctagtatgcttgtacaattttacctgatgagatcatggaagattggaagtgattgttattatttttccttctaagtttgcttcttgttctagatgacatttactgggaactatggcgagtattttggatttgccactgatgttgatgcggtggtatacttaatgctggtcaacgatctaattcatggactttatcctgatgctgtatccattggtgaagatgtaagtgcttacagtatttatgttttttagtattttattttggggatcaagctgttactactctttttgttagggtaaaatctgtcttttcataagaatgctaatttatactccctccgtctggaaatacttgtcggaggaatgaatgtatctagacgtattttagttctagatacatccatttttatgcatttctccgtcaagtatttccggacggagggagtaccttgtatggtaatgcatcacataggtaatttgagaagtgcaagggcattcaagctgacaagcatatttgttgatggctgtaatttatttgatagtatgcttgtttggatttttcagtaagtgtgagtgtgtgagtaatgttatattatttatttacttgcggaagaaatgggcaaccttgtcaattgcttcagaagactaacttagattccataaatgctgtggaaatgagaggctattcccaaggacacgaaattatacgtcagtgtgttacgcacatgtatttgtaagagcaagagcaacatggtttaacttaaattcctgcactgctatggaatctcactgtatgttgttagtgtacgcatccacaaacaagtaatcctgagctttcaactcacgagaaaataggaggctccacttctgccagcattagctgttcacagttctaatttgtgtaactctgaaattgttcaggtcagtggaatgcctacattttgcatccctgttccagatggtggtgttggttttgactatcgcctgcatatggctgtagcagataaatggatcgaactcctcaagtaagtgcaggaatattggtgattacatgcgcacaatgatctactccctctgtcccataatgtaagatgttttttgacactagtgtagtgtcaaaaaacgtcctatattatgggaaggagggagtagttcacaatttctaaattgtaaaaagaaaaatatgtatgtgaatagctagacatttccctggtatcagcttcaacacaagaagatttatcaaatacatgatttaaatagcaaatttcggaaatgtaatggctagtgtctttatgctggatattgtacatggcgctgtagcaggtgagtcaataaagctagcgatattttcagaaacaaaataatcatttatatctgtatatggggaaagtgggggtatagatggtggtcattaatcgtgttcactttttgtcctgtataagcacaggcagtaggtaataaatttagccagataaaataaatcgttattaggtttacaaaaggaatacagagggtcatgtagcatatctagttgtagttattgtaaaggctgacaagaggttcagtaaaaaaaactttatgttgatcccgggtatgcaagaacgcgagtaaagctcaaacatttatagtggttgctgttgcttgctgtatacttgtatctgcgcatatatgaaattactactacacagctgccaatctgccatgatctgtgttttgctttgtgctatttaaattttaaatgctaactcaataaatggcaataataaactaactattcaaccaatttgatggatatcagagatttcttccctcctttagtaacattgtgctcctgctgctgttctctaccgttacaaaagctgtttttccatttttcgcatcattatttttgtgtgtgagtaatttaagcatgtcctttgaagctgtgagctgttggtacttagtacattcttggtagtgtccaaatatgctgcagtctaatttagcatttctataacacaggcaaagtgacgaatcttggaaaatgggtgatattgtgcacaccctaacaaatagaaggtggcttgagaagtgcgtcacttatgcagaaagtcatgatcaagcactagttggtgacaagactattgcattctggttgatggataaggtactagctgttacttttggatcaaaagaatcacataagatttgtctcatcagattgctcatgttttcttgtgataaagatttggccccctcacccatcaccagctatttcccaactgtcacttgagcaaaacgtgccatgtggcactgtggtggcttgtgaactttgacagttaatgttgcaaatttctgttcttatttatttgattcttatgttatcgttcatttattcctcaaaaaatgttatcgttcatttgctcattcctttccgagaccagccgaagtcacgtgtagccatgtgatctgccatctgaatcttgagcaaattttatgaagaggctaaagtcgaacggattatttgcttgaatttataaatatacagacgtataatcacctggtgctttctgaaatgattaccatagtgcctgaaggctgaaatagttttggcgtttcctggacgacgcccaaaggagtgaattttattgggtagatttctggctgagccctggttacaacatacattttggagatatgcttaataacaaatctgggtgtttggtcacgagtctgcatctacatgctccttgggttttattatggcgtcatctttgtaactagtggcacccctaaggaaacattcaaaaggaaactgttacatcattctagtcaggaccaccgtactaagagcaaaattctgttccaattttatgagtttttgagactccaaaatgaacataagtgtctcatattttgctaattaactacagatgtttttatatcactttagtttttatttcaggacagttgatacttggtactgtgctgtaagcattgatccgacacagaacagcatgaacatttcgagctctctttgtgcaggatatgtatgatttcatggctctggatagaccttcaactcctcgcattgatcgtggcatagcattacataaaatgatcaggcttgtcaccatgggtttaggtggcgaaggctatcttaacttcatgggaaatgagtttgggcatcctggtcagtctttacaacattattgcattctgcatggttgtgatttactgtaatttgaaccatgctttgttttcacattgtatgtattatgtaatctgttgcttccaaggaggaagttaacttctatttacttggcagaatggatagattttccaagaggtccgcaaactcttccaaccggcaaagttctccctggaaataacaatagttatgataaatgccgccgtagatttgatcttgtaagttttagctgtgctcttacgttccctcactagatctttattggctatttatttcttgatgaaatcataatgtttgttgatcaacattgcttttgtagttttgtagacgttaacataaatatgtgttaagagttattgatcattaagaatatcatgattttttttgtagggagatgcagattttcttagatatcgtggtatgcaagagttcgaccaggcaatgcagcatcttgaggaaaaatatggggtatgtcagtatgtcactggtttgtctttgttgcatagcaagtcacagtttaacgccagtctcttcaaatggtcaaaaagtgtagaattaattcctgtaatgagatgaaaactgcgcaaaggcgggagctggaattgcttttcaccaattaaaactattttcttaagcgattgtgtattgatacctataccaacactgacaatgtaactgcagtttatgacatctgagcaccagtatgtttcacggaaacatgaggaagataaggtgatcatcttcgaaagaggagatttggtatttgttttcaacttccactggagcaatagcttttttgactaccgtgttgggtgttccaagcctgggaagtacaaggtatgcttgccttttcattgcccacccttcaccagtagggttagtgggggcttctacaacttttaattccacatgtagagtttgttgttcgtgcagctatcaatataaagaataggataatttgtaaagaaaagaatttgttgctcgagatgttgtagtcatataacatccccgaagcacatctactattcattcatattatctacttaagggtttgttacaatctttgtactcagttggactcactctaatactggaactatttaccgaatctaccctaatcatcctagcagttttagagcagccccatttggacagtccactgggtttagttggtttgtgacagtttctgctatttcttaatcaggtggccttagactccgacgatgcactctttggtggattcagcaggcttgatcatgatgtcgactacttcacaaccgtaagtctgggctcaagcgtcacttgactcgtctagactcaactgcttacaaatctgaatcaacctcccatttgctgatgcccttgcaggaacatccgcatgacaataggccgcgctctttcttggtgtacactcctagcagaactgcggtcgtgtatgcccttacagagtaagaaccagcagcggcttgttacaaggcaaagagagaactccagggagctcgtggattgtgagcgaagcgacgggcaactgcgtgaggctgctctaagcgccatgactgggaggggatcgtgcctcttcccctgatgccaggaggatcagatggataggtagcttgttgggaaaaatatggggtatgtcagtatgtcact

SEQ ID NO: 4 - protein, 823 aa, Triticum aestivum
MATFAVSGATLGVARPASAGGGLLRSGSERRGGVDLPSLLLRKKDSSRAVLSRAASPGKVLVPDGESDDLAATPAQPEELQIPEDIEEQTAEVNMTGGTAEKLQYSEPTQGIVETITDGVTKGVKELVVGEKPRVVPKPGDGQKIYEIDPTLKDFRSHLDYRYSEYKRIRAAIDQHEGGLEAFSRGYEKLGFTRSAEGITYREWAPGAHSAALVGDFNNWNPNADTMTRDDYGVWEIFLPNNADGSPAIPHGSRVKIRMDTPSGVKDSISAWIKFSVQAPGEIPFNGIYYDPPEEEKYVFQHPQPKRPESLRIYESHIGMSSPEPKINSYANFRDGVLPRIKRLGYNAVQIMAIQEHSYYASFGYHVTNFFAPSSRFGTPEDLKSLIDRAHELGLLVLMDIVHSHSSNNTLDGLNGFDGTDTHYFHGGPRGHHWMWDSRLFNYGSWEVLRFLLSNARWWLEEYKFDGFRFDGVTSMMYTHHGLQMTFTGNYGEYFGFATDVDAVVYLMLVNDLIHGLYPDAVSIGEDVSGMPTFCIPVPDGGVGFDYRLHMAVADKWIELLKQSDESWKMGDIVHTLTNRRWLEKCVTYAESHDQALVGDKTIAFWLMDKDMYDFMALDRPSTPRIDRGIALHKMIRLVTMGLGGEGYLNFMGNEFGHPEWIDFPRGPQTLPTGKVLPGNNNSYDKCRRRFDLGDADFLRYRGMQEFDQAMQHLEEKYGFMTSEHQYVSRKHEEDKVIIFERGDLVFVFNFHWSNSFFDYRVGCSKPGKYKVALDSDDALFGGFSRLDHDVDYFTTEHPHDNRPRSFLVYTPSRTAVVYALTE

SEQ ID NO: 5 - DNA, 11475 bp, Aegilops tauschii
  variation 4795: note = n is a, c, g, or t
  variation 4973: note = n is a, c, g, or t
  variation 5078..5079: note = n is a, c, g, or t
  variation 5082: note = n is a, c, g, or t
  variation 7010: note = n is a, c, g, or t
  variation 7327: note = n is a, c, g, or t
  variation 7381: note = n is a, c, g, or t
  variation 7384: note = n is a, c, g, or t
  variation 7819: note = n is a, c, g, or t
  variation 8189: note = n is a, c, g, or t
  source 1..11475: mol_type = unassigned DNA
agaaacacctccattttagattttttttttgttcttttcggacggtgggtcgtggagagattagcgtctagttttcttaaaagaacaggccatttaggccctgctttacaaaaggctcaaccagtccaaaacgtctgctaggatcaccagctgcaaagttaagcgcgagaccaccaaaacaggcgcattcgaactggacagacgctcacgcaggagcccagcaccacaggcttgagcctgacagcggacgtgagtgcgtgacacatggggtcatctatgggcgtcggagcaaggaagagagacgcacatgaacaccatgatgatgctatcaggcctgatggagggagcaaccatgcaccttttcccctctggaaattcatagctcacacttttttttaatggaagcaagagttggcaaacacatgcattttcaaacaaggaaaattaattctcaaaccaccatgacatgcaattctcaaaccatgcaccgacgagtccatgcgaggtggaaacgaagaactgaaaatcaacatcccagttgtcgagtcgagaagaggatgacactgaaagtatgcgtattacgatttcatttacatacatgtacaaatacataatgtaccctacaatttgttttttggagcagagtggtgtggtcttttttttttacacgaaaatgccatagctggcccgcatgcgtgcagatcggatgatcggtcggagacgacggacaatcagacactcaccaactgcttttgtctgggacacaataaatgtttttgtaaacaaaataaatacttataaacgagggtactagaggccgctaacggcatggccaggtaaacgcgctcccagccgttggtttgcgatctcgtcctcccgcacgcagcgtcgcctccaccgtccgtccgtcgctgccacctctgctgtgcgcgcgcacgaagggaggaagaacgaacgccgcacacacactcacacacggcacactccccgtgggtcccctttccggcttggcgtctatctcctctcccccgcccatccccatgcactgcaccgtacccgccagcttccacccccgccgcacacgttgctcccccttctcatcgcttctcaattaatatctccatcactcgggttccgcgctgcatttcggccggcgggttgagtgagatctgggcgactggctgactcaatcactacgcggggatggcgacgttcgcggtgtccggcgcgactctcggtgtggcgcgggccggcgtcggagtggcgcgggccggctcggagcggaggggcggggcggacttgccgtcgctgctcctcaggaagaaggactcctctcgtacgcctcgctctctcgaatctcccccgtctggctttggctccccttctctctcctctgcgcgcgcatggcctgttcgatgctgttccccaattgatctccatgagtgagagagatagctggattaggcgatcgcgcttcctgaacctgtattttttcccccgcggggaaatgcgttagtgtcacccaggccctggtgttaccacggctttgatcattcctcgtttcattctgatatatattttctcattctttttcttcctgttcttgctgtaactgcaagttgtggcgttttttcactattgtagtcatccttgcattttgcaggcgccgtcctgagccgcgcggcctctccagggaaggtcctggtgcctgacggcgagagcgacgacttggcaagtccggcgcaacctgaagaattacaggtacacacactcgtgccggtaaatcttcatacaatcgttattcacttaccaaatgccggatgaaaccaaccacggatgcgtcaggtttcgagcttcttctatcagcattgtgcagtactgcactgccttgttcattttgttagccttggccccgtgctggctcttgggccactgaaaaaatcagatggatgtgcattctagcaagaacttcacaacataatgcaccgtttggggtttcgtcagtctgctctacaattgctatttttcgtgctgtagatacctgaagatatcgaggagcaaacggcggaagtgaacatgacaggggggactgcagagaaacttcaatcttcagaaccgactcagggcattgtggaaacaatcactgatggtgtaaccaaaggagttaaggaactagtcgtgggggagaaaccgcgagttgtcccaaaaccaggagatgggcagaaaatatacgagattgacccaacactgaaagattttcggagccatcttgactaccggtaatgcctacccgctgctttcgctcattttgaattaaggtcctttcatcatgcaaatttggggaacatcaaagagacaaagactagggaccaccatttcatacagatcccttcgtggtctgagaatatgctgggaagtaaatgtataattgatggctacaatttgctcaaaattgcaatacgaataactgtctccgatcattacaattaaagagtggcaaactgatgaaaatgtggtggatgggttatagattttactttgctaattcctctaccaaattcctaggggggaaatctaccagttgggaaacttagtttcttatctttgtggcctttttgttttggggaaaacacattgctaaattcgaatgattttgggtatacctcggtggattcaacagatacagcgaatacaagagaattcgtgctgctattgaccaacatgaaggtggattggaagcattttctcgtggttatgaaaagcttggatttacccgcaggtaaatttaaagctttattattatgaaacgcctccactagtctaattgcatatcttataagaaaatttataattcctgttttcccctctcttttttccagtgctgaaggtatcgtctaattgcatatcttataagaaaatttatattcctgttttcccctattttccagtgctgaaggtatcacttaccgagaatgggctcctggagcgcatgttatgttcttttaagttccttaacgagacaccttccaatttattgttaatggtcactattcaccaactagcttactggacttacaaattagcttactgaatactgaccagttactataaatttatgatctggcttttgcaccctgttacagtctgcagcattagtaggtgacttcaacaattggaatccaaatgcagatactatgaccagagtatgtctacagcttggcaattttccacctttgcttcataactactgatacatctatttgtatttatttagctgtttgcacattccttaaagttgagcctcaactacatcatatcaaaatggtataatttgtcagtgtcttaagcttcagcccaaagattctactgaatttagtccatctttttgagattgaaaatgagtatattaaggatgaatgaatacgtgcaacactcccatctgcattatgtgtgcttttccatctacaatgagcatatttccatgctatcagtgaaggtttgctcctattgatgcagatatttgatatggtcttttcaggatgattatggtgtttgggagattttcctccctaacaacgctgatggatcctcagctattcctcatggctcacgtgtaaaggtaagctggccaattatttagtcgaggatgtagcattttcgaactctgcctactaagggtcccttttcctctctgttttttagatacggatggatactccatccggtgtgaaggattcaatttctgcttggatcaagttctctgtgcaggctccaggtgaaatacctttcaatggcatatattatgatccacctgaagaggtaagtatcgatctacattacattattaaatgaaatttccagtgttacagttttttaatacccacttcttactgacatgtgagtcaagacaatacttttgaatttggaagtgacatatgcattaattcaccttctaagggctaaggggcaaccaaccttggtgatgtgtgtatgcttgtgtgtgacataagatcttatagctcttttatgtgttctctgttggttaggatattccattttggccttttgtgaccatttactaaggatatttacatgcaaatgcaggagaagtatgtcttccaacatcctcaacgtaaacgaccagagtcactaaggatttatgaatcacacattggaatgagcagcccggtatgtcaataagttatttcacctgtttctggtctgatggtttattctatggattttctagttctgttatgtactgttaacatattacatggtgcattcacttgacaacctcgattttattttctaatgtcttcatattggcaagtgcaaaactttgcttcctctttgtctgcttgttcttttgtcttctgtaagatttccattgcatttggaggcagtgggcatgtgaaagtcatatctattttttttttgtcagagcatagttatatgaattccattgttgttgcaatagctcggtataatgtaaccatgttactagcttaagatttcccacttaggatgtaagaaatattgcattggagcgtctccagcaagccatttcctaccttattaatgagagagagacaaggggggggggggggggggggttcccttcattattctgcgagcgattcaaaaacttccattgttctgaggtgtacgtactgcagggatctcccattatgaagaggatatagttaattctttgtaacctacttggaaacttgagtcttgaggcatcgctaatatatactatcatcacaatacttagaggatgcatctgaanattttagtgtgatcttgcacaggaaccgaagataaattcatatgctaattttagggatgaggtgttgccaagaattaaaaggcttggatacaatgcagtgcagataatggcaatccaggagcattcatactatgcaagctttgggtattcacacaatccatttttttctgtatacacntcttcacccatttggagctattacatcctaatgcttcatgcacataaaatatttggatataatcctttattagatatatagtacaactacacttagtattctgannaanaagatcattttattgttgttggcttgttccaggtaccatgttactaatttttttgcaccaagtagccgttttggaactccagaggacttaaaatccttgatcgatagagcacatgagcttggtttgcttgttcttatggatattgttcataggtaattagtccaatttaattttagctgttttactgtttatctggtattctaaagggaaattcaggcaattatgatacattgtcaaaagctaagagtggcgaaagtgaaatgtcaaaatctagagtggcataaggaaaattggcaaaaactagagtggcaaaaataaaattttcccatcctaaatggcagggccctatcgccgaatatttttccattctatataattgtgctacgtgacttcttttttctcagatgtattaaaccagttggacatgaaatgtatttggtacatgtagtaaactgacagttccatagaatatcgttttgtaatggcaacacaatttgatgccatagatgtggattgagaagttcagatgctatcaatagaattaatcaactggccatgtactcgtggcactacatatagtttgcaagttggaaaactgacagcaatacctcactgataagtggccaggccccacttgccagcttcatactagatgttacttccctgttgaattcatttgaacatattacttaaagttcttcatttgtcctaagtcaaacttctttaagtttgaccaagtctattggaaaatatatcaacatctacaacaccaaattactttgatcagattaacaatttttattttattatattagcacatctttgatgttgtagatatcagcacatttttctatagacttggtcaaatatagagaagtttgacttaggacaaatctagaacttcaatcaatttggatcagagggaacatcaaataatatagatagatgtcaacacttcaacaaaaaaatcagaccttgtcaccatatatgcatcagaccatctgtttgctttagccacttgctttcatatttatgtgtttgtacctaatctacttttccttctacttggtttggttgattctatttcagttgcattgcttcatcaatgattttgtgtaccctgcagtcattcgtcaaataatacccttgacggtttgaatggtttcgatggcactgatacacattacttccacggtggtccacgcggccatcattggatgtgggattctcgtctattcaactatgggagttgggaagtatgtagctctgacttctgtcaccatatttggctaactgttcctgttaatctgttcttacacatgttgatattctattcttatgcaggtattgagattcttactgtcaaacgcgagatggtggcttgaagaatataagtttgatggatttcgatttgatggggtgacctccatgatgtatactcaccatggattacaagtaagtcatcaagtggtttcagtaacttttttagggcactgaaacaattgctatgcatcataacatgtatcatgatcaggacttgtgctacggagtcttagatagttccctagtatgcttgtacaattttacctgatgagatcatggaagattggaagtgattattatttattttctttctaagtttgtttcttgttctagatgacatttactgggaactatggcgaatattttggatttgctactgatgttgatgcggtagtttacttgatgctggtcaacgatctaattcatggactttatcctgatgctgtatccattggtgaagatgtaagtgcttacagtatttatgatttttaactagttaagtagttttattttggggatcagtctgttacactttttgttaggggtaaaatctctcttttcataacaatgctaatttataccttgtatgataatgcatcacttangtaatttgaaaagtgcaagggcattcaagcttacgagcatattttttgatggctgtaatttatttgatagtatgcttgtttgggtttttcaataagtgggagtgtgtgactaatgttgtattatttatttaattgcggaagaaatgggcaaccttgtcaattgcttcagaaggctaactttgattccataaacgctttggaaatgagaggctattcccaaggacatgaattatacttcagtgtgttctgtacatgtatttgtaatagtggtttaacttaaattcctgcactgctatggaatctcactgtatgttgtnagtgtacacatccacaaacaagtaatcctgagctttcaactcatgagaaaatangangtccgcttctgccagcattaactgttcacagttctaatttgtgtaactgtgaaattgttcaggtcagtggaatgcctacattttgcatccctgttccagatggtggtgttggttttgactaccgcctgcatatggctgtagcagataaatggattgaactcctcaagtaagtgcaggaatattggtgattacatgcgcacaatgatctagattacattttctaaatggtaaaaaggaaaatatgtatgtgaatatctagacatttgcctgttatcagcttgaatacgagaagtcaaatacatgatttaaatagcaaatctcggaaatgtaatggctagtgtctttatgctgggcagtgtacattgcgctgtagcaggccagtcaacacagttagcaatattttcagaaacaatattatttatatccgtatatgangaaagttagtatataaactgtggtcattaattgtgttcaccttttgtcctgtttaaggatgggcagtaggtaataaatttagccagataaaataaatcgttattaggtttacaaaaggaatatacagggtcatgtagcatatctagttgtaattaatgaaaaggctgacaaaaggctcggtaaaaaaaactttatgatgatccagatagatatgcaggaacgcgactaaagctcaaatacttattgctactacacagctgccaatctgtcatgatctgtgttctgctttgtgctatttagatttaaatactaactcgatacattggcaataataaacttaactattcaaccaatttggtggataccaganatttctgccctcttgttagtaatgatgtgctccctgctgctgttctctgccgttacaaaagctgttttcagttttttgcatcattatttttgtgtgtgagtagtttaagcatgttttttgaagctgtgagctgttggtacttaatacattcttggaagtgtccaaatatgctgcagtgtaatttagcatttctttaacacaggcaaagtgacgaatcttggaaaatgggcgatattgtgcacaccctaacaaatagaaggtggcttgagaagtgtgtaacttatgcagaaagtcatgatcaagcactagttggtgacaagactattgcattctggttgatggataaggtactagctgttacttttggacaaaagaattactccctccgttcctaaatataagtctttgtagagattccactatggaccacatagtatatagatgcattttagagtgtagattcactcattttgcttcgtatgtagtccatagtgaaatctctacagagacttatatttaggaacggagggagtacataattgatttgtctcatcagattgctagtgttttcttgtgataaagattggctgcctcacccatcaccagctatttcccaactgttacttgagcagaatttgctgaaaacgtaccatgtggtactgtggcggcttgtgaactttgacagttatgttgcaattttctgttcttatttatttgattgcttatgttaccgttcatttgctcattcctttccgagaccagccaaagtcacgtgttagctgtgtgatctgttatctgaatcttgagcaaattttattaataggctaaaatccaacgaattatttgcttgaatttaaatatacagacgtatagtcacctggctctttcttagatgattaccatagtgcctgaaggctgaaatagttttggtgtttcttggatgccgcctaaaggagtgatttttattggatagattcctggccgagtcttcgttacaacataacattttggagatatgcttagtaacagctctgggaagtttggtcacaagtctgcatctacacgctccttgaggttttattatggcgccatctttgtaactagtggcacctgtaaggaaacacattcaaaaggaaacggtcacatcattctaatcaggaccaccatactaagagcaagattctgttccaattttatgagtttttgggactccaaagggaacaaaagtgtctcatattgtgcttataactacagttgtttttataccagtgtagttttattccaggacagttgatacttggtactgtgctgtaaattatttatccgacatagaacagcatgaacatatcaagctctctttgtgcaggatatgtatgatttcatggctctggataggccttcaactcttcgcattgatcgtggcatagcattacataaaatgatcaggcttgtcaccatgggtttaggtggtgaaggctatcttaacttcatgggaaatgagtttgggcatcctggtcagtctttacaacattattgcattctgcatgattgtgatttactgtaatttgaaccatgcttttctttcacattgtatgtattatgtaatctgttgcttccaaggaggaagttaacttctatttacttggcagaatggatagattttccaagaggcccacaaactcttccaaccggcaaagttctccctggaaataacaatagttatgataaatgccgccgtagatttgatcttgtaagttttagctgtgctattacattccctcactagatctttattggccatttatttcttgatgaaatcataatgtttgttaggaaagatcaacattgcttttgtagttttgtagacgttaacataagtatgtgttgagagttgttgatcattaaaaatatcatgattttttgcagggagatgcagattttcttagatatcgtggtatgcaagagttcgatcaggcaatgcagcatcttgaggaaaaatatggggtatgtcactggtttgtctttgttgcataacaagtcacagtttaacgtcagtctcttcaagtggtaaaaaaagtgtagaattaattcctgtaatgagatgaaaactgtgcaaaggcggagctggaattgcttttcaccaaaactattttcttaagtgcttgtgtattgatacatataccagcactgacaatgtaactgcagtttatgacatctgagcaccagtatgtttcacggaaacatgaggaagataaggtgatcatcctcaaaagaggagatttggtatttgttttcaacttccactggagcaatagcttttttgactaccgtgttgggtgttccaagcctgggaagtacaaggtatgcttgccttttcattgtccacccttcaccagtagggttagtgggggcttctacaacttttaattccacatggatagagtttgttggtcgtgcagctatcaatataaagaatagggtaatttgtaaagaaaagaatttgctcgagctgttgtagccataggaaggttgttcttaacagccccgaagcacataccattcattcatattatctacttaagtgtttgtttcaatctttatgctcagttggactcggtctaatactagaactattttccgaatctaccctaaccatcctagcagttttagagcagccccatttggacaattggctgggtttttgttagttgtgacagtttctgctatttcttaatcaggtggccttggactctgacgatgcactctttggtggattcagcaggcttgatcatgatgtcgactacttcacaaccgtaagtctgggctcaagcgtcacttgactcgtcttgactcaactgcttacaaatctgaatcaacttcccaattgctgatgcccttgcaggaacatccgcatgacaacaggccgcgctctttctcggtgtacactccgagcagaactgcggtcgtgtatgcccttacagagtaagaaccagcagcggcttgttacaaggcaaagagagaactccagagagctcgtggatcgtgagcgaagcgacgggcaacggcgcgaggctgctccaagcgccatgactgggaggggatcgtgcctcttccccagatgccaggaggagcagatggataggtagcttgttggtgagcgctcgaaagaaaatggacgggcctgggtgtttgttgtgctgcactgaaccctcctcctatcttgcacattcccggttgtttttgtacatataactaataattgcccgtgcgctcaacgtgaaaatcc

SEQ ID NO: 6 - protein, 819 aa, Aegilops tauschii
MATFAVSGATLGVARAGVGVARAGSERRGGADLPSLLLRKKDSSRAVLSRAASPGKVLVPDGESDDLASPAQPEELQIPEDIEEQTAEVNMTGGTAEKLQSSEPTQGIVETITDGVTKGVKELVVGEKPRVVPKPGDGQKIYEIDPTLKDFRSHLDYRYSEYKRIRAAIDQHEGGLEAFSRGYEKLGFTRSAEGITYREWAPGAHSAALVGDFNNWNPNADTMTRDDYGVWEIFLPNNADGSSAIPHGSRVKIRMDTPSGVKDSISAWIKFSVQAPGEIPFNGIYYDPPEEEKYVFQHPQRKRPESLRIYESHIGMSSPEPKINSYANFRDEVLPRIKRLGYNAVQIMAIQEHSYYASFGYHVTNFFAPSSRFGTPEDLKSLIDRAHELGLLVLMDIVHSHSSNNTLDGLNGFDGTDTHYFHGGPRGHHWMWDSRLFNYGSWEVLRFLLSNARWWLEEYKFDGFRFDGVTSMMYTHHGLQMTFTGNYGEYFGFATDVDAVVYLMLVNDLIHGLYPDAVSIGEDVSGMPTFCIPVPDGGVGFDYRLHMAVADKWIELLKQSDESWKMGDIVHTLTNRRWLEKCVTYAESHDQALVGDKTIAFWLMDKDMYDFMALDRPSTLRIDRGIALHKMIRLVTMGLGGEGYLNFMGNEFGHPEWIDFPRGPQTLPTGKVLPGNNNSYDKCRRRFDLGDADFLRYRGMQEFDQAMQHLEEKYGFMTSEHQYVSRKHEEDKVIILKRGDLVFVFNFHWSNSFFDYRVGCSKPGKYKVALDSDDALFGGFSRLDHDVDYFTTEHPHDNRPRSFSVYTPSRTAVVYALTE

SEQ ID NO: 7 - DNA, 4431 bp, Triticum aestivum
  source 1..4431: mol_type = unassigned DNA
atggcgtcgccggcattcgcagtttccgcggcgggcctcgcccggccgtcggctcctcgatccggcgggccagagcggagggggcgcggggtggagctgcagtcgccatcgctgctcttcggccgcaacaagggcacccgttcaccccgtaattattggcgctaccttcctcactcccattctcgtttattcgtagcgggctgcggttcagcgaccttacgttccctcctggtgtggtgatgtctgtaggtgccgtcggcgtcggaggttctggatggcgcgtggtcatgcgcgcgggtgggccgtccggggaggtgatgatccctgacggcggtagtggcggaacaccgccttccatcgacggtcccgttcagttcgactctgatgatctgaaggtagtttttatttctttccttgctagtaccttcctgcatgacaattgaaatctaagacaaaaacaccatatgcgaagcctacacggtaggttggtttacaactatgtgtgccacagttcgtctgaactttttgtccttcacatcgtgttaggttccattcatcgatgatgaaacaagcctacaggatggaggtgaagatactatttggtcttcagagacaaatcaggttactgaagaaattgatgctgaaggcacgagcagaatggacaaagaatcatctacgggagagaaattacgcattctgccaccaccgggaaatggacagcaaatatacgagattgacccaacgctccgagactttaagtaccatcttgagtatcggtatgcttcgcttctattgtgtgcactttaaactttaaatacaatttacagtctttgataagatgtgaatggctgcttgctgtgacacaaaactcttgaagttcgtagtcactcttgtgtgttcatggctctgaggtgacatggtaaccgaacaaaaataggaaagtggcaagaactgcaatgtgagctaccgataagcacccattgtaattgggtacactgattaatatatgtcttgatgggttctatgttttttcagtatctatgccaattgaacaacaatgccacttcatttcccctgtgttgcttttgtaaggatgaaacccatatgtccagatcaaactgtactagcagtctcactgtgccttaatggatcaaaaacagatacagcctatataggagaatacgttcagacattgatgaacacaaaggaggcatggatgtattttcccgcggttacgagaagtttggatttgtgcgcaggtgaaatttcttgactagataagtatgtatctaccttttttctgtatcgtatctacattcctcttcccatgcagcgctgaaggtatcacttaccgagaatgggctcctggagcagatgtatgttcttctgactgtctgatcgtttacctaagtatactagttctatctttcaactgcttgtgaataattagtgctcatctgctatcctaaggttggggattttgcacttcccagatgaacagcatattaagttgcacaactagctttatttagaactaactcttgcttccaattgcagtctgcagcattagttggcgacttcaacaattgggatccaaatgcagaccatatgagcaaagtatgcatgtagtttcacaaatatataattttttctttgtaaatttgtttcttaagatctgcttactatttaaatgtggttgaatatacaccttatatgtattccggagttgagctgtgaatatagttggaagtgtttaggagtattaaagtcactagactctattctttcacttgcctgttgcacgagcccattaattactagatatcaatgttgatgatgcttttgttgtataacgtcaaattgacaacatgcatgttacccttttatataagtaatgctgcacaaatatttttgatgatttagacatgatttaatgattttggttattgcaagacactgagcggttttacatagtaatggtattggagtaggctgactggataacccgtgaactgtagctccatgtggttgatatggatttacaaatgctcatattcaatttaattgttttcagaatgacttgggtatttgggagatttttctgccaaacaatgcagacggttcgccaccaattcctcacggctcacgggtgaaggttgttttcttctcctcgccaactgtgttaggctcaggaacatgttctgtattactcacaagctcttttgaacatctaggtgcgaatgggtactccatctgggacaaaggattcaattcctgcttggatcaagtactccgtgcagactccaggagatataccatacaatggaatatattatgatcctcccgaagaggtattttacttcgtcttctgtgcttttagatttcagatatttttaattggaaagaaaattatgatttgtttttctcacgaagcttcccaagtgttatttcaagttgttctacttcttatttgttgttggcatcttagttttctattcactaaccagttatgaaattcttacatgcatgtgcaggagaagtatgtattcaagcatcctcaacctaaacgaccaaaatcattgcggatatatgaaacacatgttggcatgagtagcccggtatttcatctttaccctgtattccataaatgaagttagctatatgcaattcaagttaatttacaatttgttacaatggtatttttgtgttgttggccttctttcgttttataagtaaaaagcttatcataaatttatgtgttatgccacttggttaatacaatctgaaaaatgtaactgtggacaatctagaactagataatacaaatctgaaaaaacatgctggaatagtgtcatttcagtcaaataggatgttttgaatgctcgagagaagtactagattgtgtagcatcaaaagctggtgtccatttgttcgaacgttttacttgatgtaactgtgaatgttacatcttttgctactaaagttcattttttcactatattacatgtttcatcaacaacttaattaacctcattccttacaaacatttgtatttacatttgttcctacataaatggttattttatatatcaacttatgaatcctgaacgttataattaagaccgatggtatatcaacgattgagataatttggcatatgcggatgaattttgtggcttgttatgctcttgttttaataatataataaatagattatgcttgttggtagcctttttacattaacacatgggcaattacttgtttctttgtgcaaccaggaaccaaagatcaacacatatgcaaactttcgggatgaggtgcttccaagaattaaaagacttgggtacaatgcagtgcaaataatggcaatccaagagcactcatactatggaagctttgggtagttctctgggtcgatttctgattcttttagttattttttgtccatggaacatatttcaactttagcaactatactattatattaacttttcagctattctcttccttttcttactagtaaagtatgtgtgtgcaatgcacgtattaggtaggatattagtggcacgttatattaggtaaaatatatttatggcacattgatatttgctaagatattaattgctttcttcgcgggaatggtaaaatattaattacatgacagatttcatgggatagcgttgagtctaaacatgtttataaccaatgatagtgatgggtaattagagcgttaaacatgtttggtgctcaacattggagcgatttgaactgctagattacatgatttgacggttgagatggtttggatctgcccctttgggtctttttgtattggtatagatgtgagagactgctgcttcttgctacttcctgtgttctcattctgagtagatatcttatgagtggacaactctatgtcgacattctggaagtatcactggttgatttggtctaaaataacatactgcacagatagccacataacagtgcgattacacacataatgaccatgtttgcatagagtggcggtagtatgttcctcaccatactagcataatgatttgttatataggagtatatcatattaacttcttttccaatgacatggaagctgtaacaactttcaaatcatatttgtcttttaagtgctgcttttttcctgtttgacaattaatacaataccacttttatgtgtttttacttctattgcaggtaccatgttaccaatttctttgcaccaagtagccgttttgggtccccagaagatttaaaatctttgattgatagagctcacgagcttggcttggttgtcctcatggatgttgttcacaggtacttaatgtaatttgccgttggcgtgttaggttcacattaatcttaattctttatttcaattcctatggcctctctcctagatggaacagtaaaagc

SEQ ID NO: 8 - protein, 416 aa, Triticum aestivum
MASPAFAVSAAGLARPSAPRSGGPERRGRGVELQSPSLLFGRNKGTRSPRAVGVGGSGWRVVMRAGGPSGEVMIPDGGSGGTPPSIDGPVQFDSDDLKVPFIDDETSLQDGGEDTIWSSETNQVTEEIDAEGTSRMDKESSTGEKLRILPPPGNGQQIYEIDPTLRDFKYHLEYRYSLYRRIRSDIDEHKGGMDVFSRGYEKFGFVRSAEGITYREWAPGADSAALVGDFNNWDPNADHMSKNDLGIWEIFLPNNADGSPPIPHGSRVKVRMGTPSGTKDSIPAWIKYSVQTPGDIPYNGIYYDPPEEEKYVFKHPQPKRPKSLRIYETHVGMSSPEPKINTYANFRDEVLPRIKRLGYNAVQIMAIQEHSYYGSFGYHVTNFFAPSSRFGSPEDLKSLIDRAHELGLVVLMDVVH

SEQ ID NO: 9 - DNA, 4727 bp, Triticum aestivum
  source 1..4727: mol_type = unassigned DNA
gcctcctcatttcgctcgcgtgggtttaagcaggagacgaggcggggtcagttgggcagttaggttggatccgatccggctgcggcggcagcgacgagatggcgtcgccggcattcgcagtttccgcggcggggctcgcccggccgtcgactcctcgatccggcggggcagagcggagggggcgcggggtggagctgcagtcgccatcgctgctcttcggccgcaacaagggcacccgttcaccccgtaatttttggcgccaccttcctcactcccattctcgtttattcgcagcgggctgcggttcagcgatcttacgttccctactggtgtggtgatgtctgtaggtgccgtcagcgtcggaggttctggatggcgcgtggtcatgcgcgcgggtgggccgtccggggaggtgatgatccctgacggcggtagtggcggaacaccgccttccatcgacggtcccgttcagttcgactctgatgatctgaaggtagtttttcttttcctttttttgcatcgatctgaaggtagttgacatatattaccctgactaaactattactgccaccgtatttttatggttcgcttgaaatacctgtttacttgctacggtttgcactttcattgagacgtcagaagaaattcactgaattcctataatttggtagacaccgaaatatgtaccttttaggtcaaaatattccggcagttaagtttcagttgtatacaagaattcaaatatatatattttctcaaaatttacaactaattggtttagtttcaagtgaacgttttggtcctttggtcgagaagtaaaccgaaatcactgaaattcactgaatttcagtagtggccgaaacttttatagaactgaaattcaaaatctgctattcggcgaaattatatactaaagatttgcttatttcacacgtaggttgcggaatatcctctttctaatttgttggggaatggttcttattatcttgtcagtacctgcctgcatgacaattgaaatccaagacaaaacaccatatgcgaggcctacacgctaggttggttttacaactatgtgtgccacagttcttctgaactttttgtcttgcacattgtgttaggttgcattcatcgatgatgaaccaagcctacaggatgaaggtgaagatagtatttggtcttcagagacaaatcaggttactgaagaaattgatgttgaaggcacgaacataatggacaaagaatcatctacgggggagaaattacgcattgtgccaccaccaggaaatggacagcaaatatacgagattgacccaacgctccgagacttcaagtaccatcttgagtatcggtatgattcgcttctattgtgtgcactttaaaagaatttacagtcttcgctaagatgtgaatggctgcttgatgtatcacgaaattcttgaagttcatagtcactcttgtgtgttcatggttctgaggtaacttggtaaccgaacaaaataaggaaagtgcaagcactgcaatgtgagctactgataaccacccattgtaattgggtagactgattaatatatatgtcttcatgggctctatgtttcttttcaatatctatgccaattgaacaacaatgctttgtggacgggtgttcttttaccctctccttctatcaatagatgatacgcatactcatgcgtattctacaaaaaattgaacaacgatgccacttcatttcccccgtgttgcttttgtaaggatgaaacacatatgtccagatcaaactgtactagcagtctcactgtgccttaatggatcaaaaacagatacagcctatataggagaatacgttcagacattgatgaacacgaaggaggcatggatgtattttcccgcggttacgagaagtttggatttatgcgcaggtgaaatttcttgactaaataactatgtatctaccttttctttgtattgtatcaacattcctcttctcatgcagcgctgaaggtatcacttaccgagaatgggctcctggagcagatgtatgttcttctaaccatctgatcgtttacctaactatactagttctatctttcaactacttgtgaataattactgctcatcagctatcctaaggttggggattttgcacctcccagatgaacagcatattaagtcgcacaactagcattattaagaactaactcctgcttccaattgcagtctgcagcattagttggcgacttcaacaattgggatccaaatgcagaccatatgagcaaagtatgcatgtagtttcacaaataatattttctttgtagattagtttttttttagattggcctatctatttaaatgtggttgaatatacaccttatatgtattccagagttgagctgtaaatatagttggttggaagtgtttaggagtttaaattcactggactctattctttcacttgcctgttgcgcgagcccattactagatatcaatgttgatgatgcttttgttgtatgaggtcgaagtgaaacatgcatgttacccttttatataagtaaggttgcacatgtattttttatgatctaaacatcatttactgattttgttcttgcaagacattcagcagttttacataataatggtattggagtaggccgactgcatacctgaactgtagctccatgtggttgatatagatttacaaatgctcatattcaatgtaactgttttcagaatgacctcggtgtttgggagatttttctgccaaacaatgcagatggttcgccaccaattcctcacggctcacgggtgaaggttgtttttttctccttgccaaccgtgttaggctcaggaacatgtcttgcattactcagaagctcttttgaaaatctaggtgagaatggatactccatctgggataaaggattcaattcctgcttggatcaagtactccgtgcagactccaggagatataccatacaatggaatatattatgatcctcccgaagaggtattttacttcattttctgtgcttttagatttcagatatttttaattggaaagaaaattatgattttttttctcacgaagcttcccaattgctatttcaagctgtcctacttctatttgctgttggcatcttatttttctattcactaaccagttatgaaattccttacatgcatatgcaggagaagtatgtattcaagcatcctcaacctaagcgaccaaaatcattgcggatatatgaaacacatgttggcatgagtagcccggtatttcatctttaccctgtattccataaatgaaagttagctatatgcagtttaagttaatttacaggttgttacaatggtatttttgtgttgttgcccttctttcgttttataagtaaaaaacttatcataaatttatttgttatgccacttggttaatacaatctgaaaaatgtaactgtggacaatctagaactagataatacaaatctgaaaaaacaagctggaatagtgtcatttcagtcaaataggatgttttgaatgctcgagagaagtactagattgtgtagcatcaaaagctggtgtccatttggtcaaatgtttaacttgatgtaactgtgaatgttacatcttttgctactataagttcatatttttttcactatattacatgtttcatcaacaatttagttaacctaattccttacaaacatttgtatttaaatttgttcctacatgtatatttattttatatatcaacttataaatcctgaccgttataattaagaccaatggtatatcaatgattgagataatttggcatatgtggatgaattttgtggcttgttatgctcttgttttaataatataataaatagattatgcttgttggtagcctttttacattaacacatgggcaattacttgtttctttgtgcaaccaggaaccaaagatcaacacatatgcaaacttcagggatgaggtgcttccaagaattaaaagacttggatataatgcagtgcaaataatggcaatccaagagcactcatactatggaagctttgggtagttctctgggtcgatttctggttcttttagttattttttgtccatagaacatatttcaactttagcaactatactagtatattaacttttcagctattgtcttcctttttcttatgtgagagactgctgcttcttgctactttctgtgttctcattcagagtagacatcttatgagtggacaactctatgttgacattctggaagtatcactggttggtttggtctaaaataacatactgctcagatagccacataacagtatgattacacacacaatgaccatgtttgcatagagtggcggtagtatgttcctcaccatactagcataatgatttgttatataagagtatatcatattaacttcttttccaataacatggaagccttaacaactttcaaatcgtttttgtcttttaagtgctgcttttttcctgtttgacaattaatacaataccacttttatgtgtttctacttctattgcaggtaccatgttaccaatttctttgcaccaagtagccgttttgggtccccagaagatttaaaatcattgattgatagagctcacgagcttggcttggttgtcctcatggatgttgttcacaggtacttaatgtaatttgcggttggcgtgttaggttcacattaatcttaattctttatttcaattc

SEQ ID NO: 10 - protein, 415 aa, Triticum aestivum
MASPAFAVSAAGLARPSTPRSGGAERRGRGVELQSPSLLFGRNKGTRSPRAVSVGGSGWRVVMRAGGPSGEVMIPDGGSGGTPPSIDGPVQFDSDDLKVAFIDDEPSLQDEGEDSIWSSETNQVTEEIDVEGTNIMDKESSTGEKLRIVPPPGNGQQIYEIDPTLRDFKYHLEYRYSLYRRIRSDIDEHEGGMDVFSRGYEKFGFMRSAEGITYREWAPGADSAALVGDFNNWDPNADHMSKNDLGVWEIFLPNNADGSPPIPHGSRVKVRMDTPSGIKDSIPAWIKYSVQTPGDIPYNGIYYDPPEEEKYVFKHPQPKRPKSLRIYETHVGMSSPEPKINTANFRDEVLPRIKRLGYNAVQIMAIQEHSYYGSFGYHVTNFFAPSSRFGSPEDLKSLIDRAHELGLVVLMDVVH

SEQ ID NO: 11 - DNA, 14896 bp, Aegilops tauschii
  variation 5: note = n is a, c, g, or t
  variation 35: note = n is a, c, g, or t
  variation 45: note = n is a, c, g, or t
  variation 66: note = n is a, c, g, or t
  variation 88: note = n is a, c, g, or t
  variation 103: note = n is a, c, g, or t
  variation 107: note = n is a, c, g, or t
  variation 137: note = n is a, c, g, or t
  variation 146: note = n is a, c, g, or t
  variation 154: note = n is a, c, g, or t
  variation 156: note = n is a, c, g, or t
  variation 168: note = n is a, c, g, or t
  variation 172: note = n is a, c, g, or t
  variation 206: note = n is a, c, g, or t
  variation 210: note = n is a, c, g, or t
  variation 230: note = n is a, c, g, or t
  variation 272: note = n is a, c, g, or t
  variation 274: note = n is a, c, g, or t
  variation 313: note = n is a, c, g, or t
  variation 374: note = n is a, c, g, or t
  variation 5514: note = n is a, c, g, or t
  variation 5541: note = n is a, c, g, or t
  variation 5574: note = n is a, c, g, or t
  variation 5586: note = n is a, c, g, or t
  variation 5602: note = n is a, c, g, or t
  variation 5619: note = n is a, c, g, or t
  variation 6366: note = n is a, c, g, or t
  variation 6596: note = n is a, c, g, or t
  variation 6604: note = n is a, c, g, or t
  variation 6623: note = n is a, c, g, or t
  variation 6676: note = n is a, c, g, or t
  variation 6746: note = n is a, c, g, or t
  variation 6787: note = n is a, c, g, or t
  variation 6803: note = n is a, c, g, or t
  variation 6845: note = n is a, c, g, or t
  variation 6864: note = n is a, c, g, or t
  variation 6877: note = n is a, c, g, or t
  variation 6963: note = n is a, c, g, or t
  variation 7055: note = n is a, c, g, or t
  variation 7123: note = n is a, c, g, or t
  variation 9161: note = n is a, c, g, or t
  variation 9206: note = n is a, c, g, or t
  variation 9284: note = n is a, c, g, or t
  variation 10857: note = n is a, c, g, or t
  variation 10861: note = n is a, c, g, or t
  variation 10926: note = n is a, c, g, or t
  variation 11177: note = n is a, c, g, or t
  variation 11224: note = n is a, c, g, or t
  variation 11336: note = n is a, c, g, or t
  variation 12749: note = n is a, c, g, or t
  variation 12771: note = n is a, c, g, or t
  variation 12964: note = n is a, c, g, or t
  variation 12984: note = n is a, c, g, or t
  variation 12986: note = n is a, c, g, or t
  variation 13126: note = n is a, c, g, or t
  variation 13893: note = n is a, c, g, or t
  variation 14086: note = n is a, c, g, or t
  variation 14109: note = n is a, c, g, or t
  variation 14156: note = n is a, c, g, or t
  variation 14173: note = n is a, c, g, or t
  variation 14517: note = n is a, c, g, or t
  variation 14591: note = n is a, c, g, or t
  source 1..14896: mol_type = unassigned DNA
ttttncggggttgcttcttccagattcatattcgnaaaatttcanatggagcttaaaaataatgtngttgagcttttttgagggtttncaaatggccaattanaacnttgggttttgaattaattgaatccaatttngaccaattnatttaagnanttttaaattggnatgnaaaacttttatttgaactattgtcgccttggaantttnattttttgagtttaaaaggncttgcgatatttttcttttgattgttttccaacccatgtaananagtgcgttaaaagagcaaaggtatacgccaaagaaggcncacccattacaaagccacataggcatgacccaactaaagaacccaaaccattgatgattcncaattaataaacccaccaaaatcagcttaaattagagcaagggcattacacatgcaacaaagtaggcaacattttcagtgcataagactacaatagacaacaacactatctcaacacgaccacgacaacggacaacatcgaataaaacccaaagaacacatgaagaaggcatgacgtcactgagcaaggaggctacaaatccaccaccgccgggccgacttgccaccaaggccatagaaggaccgtggggtgcataggcataaaccagagcaggaggcacatgaccaacaaagacaacatcaaccacgatcaagtaccacaacaggggaggagtgagggaaaatgccatcggagactcgaagtggacgttggagtatttttctttgattattttcaatattcaaactacacaagatcaacaacagatgagagaccaaaacatttgataactacagttggataatattggccatgatgtctgtttgatgatccgcccgagatgccaagctttgtagccttgcacgggctccccaacaaactgcctcactcgattgtcaaaaaagtaaaaatgattgtagaaaaaaaaactgactcactcgtcactaccctaccgtcctacatgacacctggccgcaagacgacgccgtcctcctgccgcgcgcgtccgcgatcacaccaccgcaaaaaccaaaacctcttcgccggtgcgtcccacgctaccatccatgcagccgtccgcccgcgcgcgcgttgcccgcaccacccgctggcggccaccacgccgccactctcgcgtgaaggctccgtccgcttcctcctagttccactctctctccgtgctagcagtatatagcatccgccctccgccccctcccaatcttagaacacccctccctttgcctcctcatttcgctcgcgtgggtttaagcaggagacgaggcggggtcagttgggcagttaggttggatccgatccggctgcggcggcggcgacgggatggctgcgccggcattcgcagtttccgcggcggggctggcccggccgtcggctcctcgatccggcggggcagagcggagggggcgcggggtggagctgcagtcgccatcgctgctcttcggccgcaacaagggcacccgttcaccccgtaattatttgcgccacctttctcactcacattctctcgtgtattctgtcgtgctcgcccttcgccgacgacgcgtgccgattccgtatcgggctgcggtgttcagcgatcttacgtcggttccctcctggtgtggtgatgtctgtaggtgccgtcggcgtcggaggttctggatggcgcgtggtcatgcgcgcgggggggccgtccggggaggtgatgatccctgacggcggtagtggcggaacaccgccttccatcgacggtcccgttcagttcgattctgatgatctgaaggtagttttttttttgcatcgatctgaaggtacttgacatatactactgtattaccctgagtaaatactgccaccatatttttatggttcgcttgaaatacctgtttacttgctacggttttcactttcattgagacgtcggacgaaattcactgaattcctataatttggtagacaccgaaatatatactactccttccgtcccataatataagagcgtttttggcaccttatattatagggcggagggagtaccttttaggtcaaaatattgtggtagtttcaattgtatacaagaattcaaatattttttttaaaaaaaaatcaactaattggttgagtttcaagtgaagcgttttggtcctttggctgagatgtaaaccgaaatcactgaaattcatagtagccgaaactttaatagaactgaaactcaaaatctgctatccggcgaaattctaaagatttgcttatttcacacgtaggttgcagtacaccctctttctaatttattggggaaggggtattattatcttgttagtacctgcctgcatgacaattgaaatctaagacaaaacaccatatgcgaggcctacacacggtaggttggtttacaactatgtgtgccacagttcgtctgaactttttgtccttcacatcgtgttaggttccattcattgatgatgaaacaagcctacaggatggaggtgaagatagtatttggtcttcagagacaaatcaggttagtgaagaaattgatgctgaagacacgagcagaatggacaaagaatcatctacgagggagaaattacgcattctgccaccaccgggaaatggacagcaaatatacgagattgacccaacgctccgagactttaagtaccatcttgagtatcggtatgcttcgcttctattgtgtgcactttaaaaacaatttacagtctttgataagatgtgaatggctgcttgctgtgacacgaaactcttgaagttcgtagtcactcttgtgtgttcatggttctgaggtaacatggtaaccgaacaaaaataggaaagtggcaagcactgcaatgtgagctactgataaccacccattgtaattgggtacactgattaatatatatgtcttcatgggctctattttttttcaatatctatgccaattgaacaacaatgctttgtggacgggtgttcttttaccctcttcttctatcaatagatgatatgcatactcatgcgtatcctacaaaaaattgaacaacaatgccactttcccccgtgttgcttttgtaaggatgaaacacatatgtccagatcaaactatactagcagtctaactgtgccttaatggatcaaaaacagatatagcctatacaggagaatacgttcagacattgatgaacacgaaggaggcatggatgtattttcccgcggttacgagaagtttggatttatgcgcaggtgaaatttcttgactaaataactatgtatctaccttttctttgtactctatcaacattcctcttcccatgcagcgctgaaggtatcacttaccgagaatgggctcctggagcagatgtacgttcttctaaccatctgatcgtttacctgactatactaattctatctttcaactaattgtgaataattactgctcatcagctatcctaaggttggggattttgcacctcccagatgaacagcatattaagtcgcacaactagcattattaagaactaactcctgcttccaattgcagtctgcagcattagttggcgacttcaacaattgggatccaaatgcagaccatatgagcaaagtatgcatgtagtttcacaaatatatcatattttctttgtagatttttttttttagatcggcttatctatttaaatgtggttgaatatacaccttatatgtacgttgagctgtaaatatagttggaagtgtttaggagtattaaattcactggactctattctttcacttgcctgttgcacgagcccattactagatatcaatgttgatgatgcttttgttgtatgaggtcgaagtgaaacatgcatgttacccttttatataagtaaggttgcacatgtattttttatgatctaaacattatttactgattttgttcttgcaagacactaagcagttttacataataatggcgttggagcaggccgactgcacatctgaactgtagctccatgtggttgatatagattacaaatgctcatattcaatgtaactgttttcagaatgaccttggtgtttgggagatttttctgccaaacaatgcagatggttcgccaccaattcctcacggctcacgggtgaaggttgttttcttctccttgccaacggtgttaggctcaggaacatgtcctgtattactcagaagctcttttgaacatctaggtgagaatggatactccatctgggataaaggattcaattcctgcttggatcaagtactccgtgcagactccaggagatataccatacaatggaatatattatgatcctcccgaagaggtattttacttcatcttctgtgcttttagatttcagatatttttattagaagaaaattatgattttttccctcacgaaccttcccaattgctatttcaagctgtcctacttatttgctgctggcatcttatttttctattctctaaccagttatgaaattccttacatgcatatgcaggagaagtatgtattcaagcatcctcaacctaaacgaccaaaatcattgcggatatatgaaacacatgttggcatgagtagcccggtatttcatctttaccatgtattccataaatgaagttagctatatgcagttcaaatttatttacaggttgttacaatggtatttttgtgttggtgcccttctttcgttttataagtaaaaaacttatcataaatttatttgttatgccgcttggttaatacaatctgaaaaatgtaactgtggacaatctagaactagataatacaaatctgaaaaaacatgctggaatagtgtcatttcagtcaactaggatgttttgaatgctcaagagaagtactagtgtgtagcatcaaaagctggtgtccatttgttcaaatgtttaattaacactatagtgaaaacaagtaattgcacaaagaaacaagtaattgcccaagttcatatgttttttcactatattacatgtttcatcaacaatttaattaacctcattccttacaaacatttgtatttacatttgttcctacatatatagttattttatatatcaactttataaatcatgactgttataattaaaaccgatggtatatcaacgattgagataatttggcatatgtggatgaattttgtggcttgttatgctcttgttttaataacataataaatagattatgcttgttggtagcctttttacattaacacatgggcaattacttgtttctttgtgcaaccaggaaccaaagatcgacacatatgcaaacttcagggatgaggtgcttccaagaattaaaagacttggatacaatgcagtgcaaataatggcaatccaagagcactcatactatggaagctttgggtagttctctgggtcgatttctggttcttttagttatcttttgtccatagaacatatttcaactttagcaactatactattatattaacttttcagctattgtcttnctttttcttatgtgagagactgctgcntcttgctacttcctgtgttctcattcagagtanacatcttatgantagacaactctatgtngacattccggaagtatncactggctgattcggtctaaaataacatactgctcagatagccacataacagtacgattacacacataatgaccatgtttgcatagagtggcggtagtatgttcctcaccatactagcataatgacttgttatataagagtatatcatattaacttcttttccaatgacatggaagctgtaacaactttcaaatcatttttgtcttttaagtgctgcttttttcctgtttgacaattaatacaataccacttttatgtgtttttacttctattgcaggtaccatgttaccaatttctttgcaccaagtagccgttttgggtccccagaagatttaaaatctttgattgatagagctcacgagcttggcttggttgtcctcatggatgttgttcacaggtacttaatgtaatttgaggttggcgtgttaagttcacattaatcttaattctttatttcaattcctatggcctctctcctagattggaacagtaaaagcatcatccagtttgtataaattgctaaaagaacattttacatgttaagtattttcaattactatgaaacatataaatttacatacttattgattttacgacagaagtaccgatctcacaagatgaacaattggttgatcacatatcatttcatactacaatacaagaaaatgaatagagaacgagttaatattagccttggtaaaatcagcaacttgtttggaaataaagtatagtgatgccagtgcaaanaacaaggcatcaagttggtttcagctcccacggtcggtgctagctgtcaagggtaatttgcacgtagtcgcacatagatttgtgtgggagtggaaagtaaccacagattgtccgaggaacacgggacacacgtcttagccacaggtttgggctccccttgatgcgggtagtagctttactccttatatgaaattatctcaagatagatttcaatttggggttacacttangaactcancaagttaaggatcaactcnctgagttctatacgactgatctttgaccgagatatcttgatcaggctaagtancaaaatccaggccttgagatgttgaacatgtccttcattttgggctgggtgcccttgggcataaggtgtngtccttccttcatgtgcttcttgcagcgtatgacataaacntcctctgagttggtanatgcacggttccctttgaggaaatcaggggtagtcgcatctngggaaagttggtcacccangcatggatcctcngcgcacaccgggcaaacacggtgaaaccacttctcctcgacactagctaacttgacattcaagcaaactaagaatataactttatntctaaatgaaccggacaccctccttgtgcctgcacctacagagtacaatgccagttttggactgaactcttgtgttcatgtatgtgctaatnacataggttctaaccatgattctaaatagcgcgttataactccactatagtaatgctatagcgtttanaagatcccgcactaagggaccttagtccaaatacatgatcaaacattttacatagcgcgctatagctatttaaaactatggtcacccgctaagaggcataactcgctatttaaaactatggttctaacttttaatctattttatgtcttggtccaaagcccctttttgttctatagctttacctttgggttgagatcacccttaacccattggtaatcctggttgatttactccatcctttcttgcgtagctttacttttggttttttgtttctcacagtcacgcgtcaaataataccttggacgggttgaatggttttgatggcacggatacacattacttccatggcggttcacggggccatcactggatgtgggattcccgtgtgtttaactatgggaataaggaagtatgggactatagaatttctattgccatttgttatgtatttatccattaattaatcctccaaccgatattccaacattgttatctttatacaggttataaggtttctactttccaatgcaagatggtggctagaggagtataagtttgatggtttccgattcgatggcgcgacctccatgatgtatacccatcatggattacaagtaattcattgcttgattgtctttgttctatcttgactacctgtgcaactttaataagattacgcctagctaatattttcttttatgttatagtatcaatttttatttgagcttgaaacctaaattactttttttttgaattgctgcgctctattttaggtaacctttacaggaagctaccatgaatattttggctttgccactgatgtagatgcggtcgtttacttgatgctgatgaatgatctaattcatgggttttatcctgaagccgtaactatcggtgaagatgtaagtgtttctatagtcatctttcaatatgaatttgttagaactattggtacttatcttttttgtagtttaggctattctgttcattcttacaggaggtgcatacagaagttgctttagattttgaaacgcagtgcacattgtgccattactttgtagctatatcgagttgagacttgagagccatggtaatcaagttcctgacgtggcattgcattagatagttgcatgtctaagttcctgacgtggagatagaagaaagaacgcaccccccgcgtcgctcctctcagggcgacacgggcggagccctcacccccgccgccacagggagcatccacccttctcctctcccctcgccgccgccggagggcaaagaccgcgcggcgtcgcggcggtgggtgcggcctgggctggcatctggcagcggcgatttggcctcccctgcccagaactgtgctgccgcggtttgtggcagcttgggcatcggcagtggcccgagtctgcggtggcggcgtgtctggcgtccggaggtgcagcgattgtgcggttgtgtggctcaggctcggagggcgtgcgggctgccaggtccggccagatctggcctcgagtggcttcgtacggggcggtggctgttgcgggtccgtgggccgaggttcgggtgtggctgctgcttgcccggaccggtggtgcgtaacgatgccggagcagcgtcctcgggtcgttgaagtgggcgctcctccggcagcttcaggtggtgattcgtcgcagcgggtggtgcactgggggtctcggctgattgtggtgccatggtggtggtggttgttggcggtagcaaagtgcctggtgcacacggctagggttttggcggatggacagacttgatgcaatgccttagggcatagtgaatttcagctaagtacctagcaccgaccttggtcaatgccgccgccgctggtgtcttaggacgttgttgcccttgttggaggcgtgttgtggagccccttcacctccatgggcatttagatctcgagctctctgggtgaaaacgccggctttggctttggccggagtgggcggtggcggcgtaaccgtcgctccccccatgggggtgtagtcttggaggtctagacttctgntaagcgtcagtggttggtgtggtcctgaaggttcgtatcggctangcaggagcacggtctcagggccggtgtggaagccagagcagcagctccggagagcgcatttgtgttgcatggtgcccnagtctggtcgcttgggtggcttgaacccatccggttcagtgggtacacagccttggggctggtgtgtggagaacgcctttatgttatagggtatcaattttgttcacttgggttacggagtcgtcgactcgtctggtacacggcctcagggccgatatgtgtctctctgtgtgtgtctgtttgtgtgttgttgaggtttgtacgccagggcggcggctccaagtcgtgttgtatggtatcgactctggtcgttagagcgactgagtcgccggctcatttggggcgcagcctcggaactggtgtgtgtgtcacaggctcacaactgtattagttttgagctagttttccttgttaaccggtcaattaaaattcttctgtatgaaaaggcagagctactgtcagttactagggaaaaaatgtttctggcatggaaaactattttctatccatttcatgtagtgacaacttttctttttcttgagtgagactactaactttccatgaaagtcagatgaaaatcaacaacttctataaacaaacagaactttccagaaataaggaacaaattgttggatatatcagcaatttttcaatttatttatttaatacgaaagcatgatgatagtgctggcaagatttaatcctaattgtaatctaaacatgtgagtgcgtgcataaaacatgcatatctcttaacatagtgagtactggaaactcatgaaccaagcagaagtgggatgaatggatcataccctccagtagcaaaagtaaggggttagggccgtggcaacagcagcattggcattggtggccttcttcaaggaaccattgttcccacccatgggtttggttggggaagtcaagaaagtagtcgaagtcgtggatgcaaaaggacagaagggagccgtcagggtgacatgctccccaataaccttattgatctccacctggtgcatggttctgttgcgtgccgcagagaaggtgcgcacatgtacccactcctcttctcatgctcccaatgggtcatgaagagagttcttatatattggtccaaattctcctccactccttgggtcggactaaatttccaaccatttcatgaaaccactaatgggtctttgagattatgcaggaattattaattatataatatggcccaaggcccatctaactttcaacaataataacttaattgttcaactgaggtgttggtttttcatttgaattctcaggttagtggaatgcctacatttgcccttcctgttcaagttggtggggttggttttgactatcgcttacatatggctgttgccgacaaatggattgaacttctcaagtaagtgtttcaaaattggtatgcatatgttaatattttactggacagaagatttgattgtcagtgtatattaatgcaattaaaatgttcctttgcgtaacactattgcacatatggacttccacatgaatgtccaaaaacatgtatcgttattagtgtatttgatttggccacaatgtgattatagttgtgatttcgtagtttatacagtataacaacaaaagtaggatacatgtnccancttttgggaattcttataatgatattacacttttttaatcttgcatccctcatcatttctattgnctcagttgtttcaagtttctataaaagtttggtttcgtgttctggttattgatgtggagtatcttgtatctgaaacatgaattgcaactttttattctaaacagaggaaacgatgaagcttgggagatgggtaatattgtgcacacactaacaaacagaaggtggctggaaaagtgtgttacttatgctgaaagtcacgatcaagcacttgttggagacaagactattgcattctggttgatggacaaggnaacaacacattatttctccagactttaaatactaacatttattttgnttcgcaatttccttatatctatgatttttaattatacttatctctcttgattttcctccccacaaaaatgcaagctagaatttttttcctcatgaaagtatgcaaagcttgngcctatgcattgataaagtatttacaagcctaagaataggcgacaaccgaccactcaagcgaagtccacaatcaaatggttgtcagacacgttaccgaactacttttagcctaaatgacaaccacacaaacacactgggttgcccttgctttggataaatcgatggcccctagactgccggtcgcacctcggaactgccaaccgccatggccgcttgaatagtgaaccatcacacattgccatatcctctacatgttgaaacttagtacgcaaccgtagtcgatcgtgcccccaagatgccacatcacgggattggcacatggctgatgcaagtaggaaccactgaaaaagccaaggcttgtgttcataccaatcgaaggaaaaccttgaagaacaaagccattagaaaggtatcaacatcaactggctacctcgtctgatctaggtctgaatcgacaaacccgatttggcttttcactctaaggattagacaacaggggatggggtaaattggtattccttgacaatgcccccaaggtggaaacggtgttaggaaggcgtcactagtccttttatacagactgctaagtgcggagacgggaatcgaacccgtgacctcaaggttatgagcctcgtcggcattgacagtgggaatatcaagtgcccctaacacttaggtgttcccatgtctagaaaaatcaattttaaatgtttcaaaaaaaatctttttttgtgaatcttcataaaacatgtgtttgcaacccctaaaaagtacaaatccaaactcagaatacatataggacaacacaaaagacaaatctagatgtgaacattgccatttttgttttttgattctattcatgatgatttgtctttttcgttcctccaagtattttgattttaattttttagaggtcgtagacacatcctatgaactttcacaactctttttctagaatcgtttgaaacgtttaaaaatgaatctctagatatggggaatgacaacgcccaagtgttgggagcactatttttttttcccacacctacctggtggggatgggtttcgtttgaagccgcatgaccaccactcatgccatgggtgcagccccatgaaggctcctcaacaaaatgtatcaccacccgcaatcacactagctcgacagcctgattgccatgccatcccaccaacaaggactaccatgcattgcacatcaggaccaacatagactgactgcaaggagctggaacatggtttaggagcctatgtacttgaaacctatacctgcagtgagatgcccggacctacgacaacaccactagagcacacatcagcaaccctgggcaaagcatgacaaatgatgccaggtacatccatccgtagatgttgcaagatgacgccggacgggaactaanagcatcttcaataacttgtggnatgttagtttgttacaaaatatagtaatctcttcaccaataagccattctacaaatactccaatggagtgtatttagcttgtcgaataggaggtgagagaatatataggattgctcttaggtatcgctaagtgatgtaagcgcaagccctatggttgtcccaatcttcataatttgtaggtggcaaaggggancatcacaaagaacacatagncntaaagaggaaacacccaaacaatattctcatcacacatgtcctctttagtttaatgctttgctaaccctaatttgagtccatggtacagtttatacagcatagggacgaagctggtaggtaggagagggatacatggccncttgactcgatttccacgcaggcatgggacgggcagtccaggttgcaagcctgacagtctggtcgttgtagctcgtccggatcagggcttccccatgcgccgctcgaagggctcactccaggtgtcgatagtctaggctccaagttggacagccggcttcaccaatttagcttttgttgcctagcgtgtggtagccggactaaggaccagacggtgttctagagaaggtttttgtctggttgttggattccagtagcccatctccttcgtcgtgcacttcctccttcgcttccacgcttccttcgcagatggtgtaggcgtactcttgtgcttgccttggtaaattcgctctcatgttcaatatggacgaactcattgccacacgccgccatgtttgaagggtaagtcccatgctcaaccatctcatcaccgttgccaaggatgaaggccatatggtgtggctcatcatcatcatccgcctccatgactgaagggaatatcccatgctcaaccatctcacttgaggtgcctccgaagatggagcggtcacacttgcttgttgttggtgatcgacgtgatgtagtcgccggagcgtcagagcttgaaggagtatcacttatttgtaactcgaggaagagaaatgttgcttgatgtagcattgtgtccattgttggtgtagattcttcaaggaatgttttcttgtaggctttatgcatcgttttcttgatgtcgtggccattcctcttcatgatgcgtttggagtgtgnaagcgctcttcttgttgatgatgttcacggtcaagggtgtggccttggaatccatcgttgcgaagaaggttgtagcttgatgttgctcttgtactttgaggtgtcaatgccggtgtgatcctgaagacttgtggtggttgagcacgttttgaaagagttgtgcgtcaatcttggcgtcccaatggtccatctnggcgtcaaagtttttggtgtagncctagaccggaggtgatgtgccttctctattcatattgacactcgancaaagtgtgagtggacnaagggaaagaacaataccaaagttacctctttccgatgttggtgaaggatcaagcgatctcacactatggaatattaagagagaacaataccaaagttacctctttccgatacattcgtcaacacaatacctttgtcgaggttggaggcaaccggccttgatttcggttgtggtgtcaaaggatggagtggttgttgttgttacgagaaccaaagcggaagaacaaccacaaatcaaaaaaggggcgaaagatgacaaatttcagcagattcggaagaggtcggacagtcgagttggtggcccctttttcgactcgagcaaagtgtgagtggcaagggaaagancaataccaaagttacctctttccgatgttggtgaaggatcaagcgatctcacactatggaatattaagagagancaataccaaagttacctcttcccgatacattggtcaacacaatacctttgtcgaggtcagaggcaaccggccttgctttcggttgtggtgtcaaaggatggagtggttgttgttgttacgagaaccaaagcggaagaacaaccacaaatcgaaaaaggggcgaaagatgaacaaatttcagcagattcaagagaggtcggacagtccgagttggtggccggacgggggttggctggacagtccgggttggaagctgacagttcgggtaggtcaactcggctgttcttcaggggaaattggatcga

SEQ ID NO: 12 - protein, 623 aa, Aegilops tauschii
MAAPAFAVSAAGLARPSAPRSGGAERRGRGVELQSPSLLFGRNKGTRSPRAVGVGGSGWRVVMRAGGPSGEVMIPDGGSGGTPPSIDGPVQFDSDDLKVPFIDDETSLQDGGEDSIWSSETNQVSEEIDAEDTSRMDKESSTREKLRILPPPGNGQQIYEIDPTLRDFKYHLEYRYSLYRRIRSDIDEHEGGMDVFSRGYEKFGFMRSAEGITYREWAPGADSAALVGDFNNWDPNADHMSKNDLGVWEIFLPNNADGSPPIPHGSRVKVRMDTPSGIKDSIPAWIKYSVQTPGDIPYNGIYYDPPEEEKYVFKHPQPKRPKSLRIYETHVGMSSPEPKIDTYANFRDEVLPRIKRLGYNAVQIMAIQEHSYYGSFGYHVTNFFAPSSRFGSPEDLKSLIDRAHELGLVVLMDVVHSHASNNTLDGLNGFDGTDTHYFHGGSRGHHWMWDSRVFNYGNKEVIRFLLSNARWWLEEYKFDGFRFDGATSMMYTHHGLQVTFTGSYHEYFGFATDVDAVVYLMLMNDLIHGFYPEAVTIGEDVSGMPTFALPVQVGGVGFDYRLHMAVADKWIELLKGNDEAWEMGNIVHTLTNRRWLEKCVTYAESHDQALVGDKTIAFWLMDK

SEQ ID NO: 13 - DNA, 23 bp, synthetic construct
  misc_feature 1..23: note = Synthetic Construct
  source 1..23: mol_type = other DNA
acggctttgatcatctcctccca

SEQ ID NO: 14 - DNA, 25 bp, synthetic construct
  misc_feature 1..25: note = Synthetic Construct
  source 1..25: mol_type = other DNA
tttgtctctttgatgttccccaaat

SEQ ID NO: 15 - DNA, 31 bp, synthetic construct
  misc_feature 1..31: note = Synthetic Construct
  source 1..31: mol_type = other DNA
tatgaccagagtatgtctacagcttggcaat

SEQ ID NO: 16 - DNA, 27 bp, synthetic construct
  misc_feature 1..27: note = Synthetic Construct
  source 1..27: mol_type = other DNA
tgcatcctaagtgggaaaccctaacca

SEQ ID NO: 17 - DNA, 27 bp, synthetic construct
  misc_feature 1..27: note = Synthetic Construct
  source 1..27: mol_type = other DNA
tcaatttggatcagaggggatagtcca

SEQ ID NO: 18 - DNA, 27 bp, synthetic construct
  misc_feature 1..27: note = Synthetic Construct
  source 1..27: mol_type = other DNA
tgacaaggttgcccatttctaatgcaa

SEQ ID NO: 19 - DNA, 28 bp, synthetic construct
  misc_feature 1..28: note = Synthetic Construct
  source 1..28: mol_type = other DNA
gatagctggattaggcgatcgcctcagg

SEQ ID NO: 20 - DNA, 30 bp, synthetic construct
  misc_feature 1..30: note = Synthetic Construct
  source 1..30: mol_type = other DNA
ttggtagaggaattagcaaagtaaaatcca

SEQ ID NO: 21 - DNA, 32 bp, synthetic construct
  misc_feature 1..32: note = Synthetic Construct
  source 1..32: mol_type = other DNA
ggtagaaccttttgcattatgtgtgcttttcc

SEQ ID NO: 22 - DNA, 33 bp, synthetic construct
  misc_feature 1..33: note = Synthetic Construct
  source 1..33: mol_type = other DNA
gctacctcgaaatgcaatggaaatcttagagac

SEQ ID NO: 23 - DNA, 27 bp, synthetic construct
  misc_feature 1..27: note = Synthetic Construct
  source 1..27: mol_type = other DNA
ccaaggagggagtgaggagcttgactt

SEQ ID NO: 24 - DNA, 27 bp, synthetic construct
  misc_feature 1..27: note = Synthetic Construct
  source 1..27: mol_type = other DNA
tgtcagcttgaatgcccttgcacttct

SEQ ID NO: 25 - DNA, 23 bp, synthetic construct
  misc_feature 1..23: note = Synthetic Construct
  source 1..23: mol_type = other DNA
gatcgcgcttcctgaacctgtat

SEQ ID NO: 26 - DNA, 25 bp, synthetic construct
  misc_feature 1..25: note = Synthetic Construct
  source 1..25: mol_type = other DNA
ctcagaccacgaagggatctgtatg

SEQ ID NO: 27 - DNA, 27 bp, synthetic construct
  misc_feature 1..27: note = Synthetic Construct
  source 1..27: mol_type = other DNA
atgaatacgtgcaacactcccatctgc

SEQ ID NO: 28 - DNA, 29 bp, synthetic construct
  misc_feature 1..29: note = Synthetic construct
  source 1..29: mol_type = other DNA
ggaagcaaagttttgcacttgccaatatg

SEQ ID NO: 29 - DNA, 28 bp, synthetic construct
  misc_feature 1..28: note = Synthetic Construct
  source 1..28: mol_type = other DNA
cgtctccagcaagccatttcctacctta

SEQ ID NO: 30 - DNA, 28 bp, synthetic construct
  misc_feature 1..28: note = Synthetic Construct
  source 1..28: mol_type = other DNA
ttttgccactagtttttgccaattttcc

SEQ ID NO: 31 - DNA, 29 bp, synthetic construct
  misc_feature 1..29: note = Synthetic Construct
  source 1..29: mol_type = other DNA
tcaatcaatttggatcagagggaacatca

SEQ ID NO: 32 - DNA, 36 bp, synthetic construct
  misc_feature 1..36: note = Synthetic Construct
  source 1..36: mol_type = other DNA
tagcagtgcaggaatttaagttaaaccactattaca

SEQ ID NO: 33 - DNA, 24 bp, synthetic construct
  misc_feature 1..24: note = Synthetic Construct
  source 1..24: mol_type = other DNA
ctcccattctcgtttattcgtagc

SEQ ID NO: 34 - DNA, 26 bp, synthetic construct
  misc_feature 1..26: note = Synthetic Construct
  source 1..26: mol_type = other DNA
gttcggttaccatgtcacctcagagc

SEQ ID NO: 35 - DNA, 28 bp, synthetic construct
  misc_feature 1..28: note = Synthetic Construct
  source 1..28: mol_type = other DNA
gccaattgaacaacaatgccacttcatt

SEQ ID NO: 36 - DNA, 24 bp, synthetic construct
  misc_feature 1..24: note = Synthetic Construct
  source 1..24: mol_type = other DNA
gagtacccattcgcacctagatgt

SEQ ID NO: 37 - DNA, 27 bp, synthetic construct
  misc_feature 1..27: note = Synthetic Construct
  source 1..27: mol_type = other DNA
gcctgttgcacgagcccattaattact

SEQ ID NO: 38 - DNA, 28 bp, synthetic construct
  misc_feature 1..28: note = Synthetic Construct
  source 1..28: mol_type = other DNA
ttcgaacaaatggacaccagcttttgat

SEQ ID NO: 39 - DNA, 28 bp, synthetic construct
  misc_feature 1..28: note = Synthetic Construct
  source 1..28: mol_type = other DNA
ttatatatcaacttatgaatcctgaacg

SEQ ID NO: 40 - DNA, 31 bp, synthetic construct
  misc_feature 1..31: note = Synthetic Construct
  source 1..31: mol_type = other DNA
gtaaagtgttcttttagcaatttatacaaac

SEQ ID NO: 41 - DNA, 30 bp, synthetic construct
  misc_feature 1..30: note = Synthetic Construct
  source 1..30: mol_type = other DNA
gcctcctcatttcgctcgcgtgggtttaag

SEQ ID NO: 42 - DNA, 36 bp, synthetic construct
  misc_feature 1..36: note = Synthetic Construct
  source 1..36: mol_type = other DNA
agtgactatgaacttcaagaatttcgtgatacatca

SEQ ID NO: 43 - DNA, 32 bp, synthetic construct
  misc_feature 1..32: note = Synthetic Construct
  source 1..32: mol_type = other DNA
ctacaaaaaattgaacaacgatgccacttcat

SEQ ID NO: 44 - DNA, 27 bp, synthetic construct
  misc_feature 1..27: note = Synthetic Construct
  source 1..27: mol_type = other DNA
ccaactatatttacagctcaactctgg

SEQ ID NO: 45 - DNA, 27 bp, synthetic construct
  misc_feature 1..27: note = Synthetic Construct
  source 1..27: mol_type = other DNA
actgattttgttcttgcaagacattca

SEQ ID NO: 46 - DNA, 24 bp, synthetic construct
  misc_feature 1..24: note = Synthetic Construct
  source 1..24: mol_type = other DNA
caaatggacaccagcttttgatgc

SEQ ID NO: 47 - DNA, 37 bp, synthetic construct
  misc_feature 1..37: note = Synthetic Construct
  source 1..37: mol_type = other DNA
aaagttagctatatgcagtttaagttaatttacaggt

SEQ ID NO: 48 - DNA, 30 bp, synthetic construct
  misc_feature 1..30: note = Synthetic Construct
  source 1..30: mol_type = other DNA
tgtaagatgttctttcagcaatttatacta

SEQ ID NO: 49 - DNA, 22 bp, synthetic construct
  misc_feature 1..22: note = Synthetic Construct
  source 1..22: mol_type = other DNA
acgacgcgtgccgattccgtat

SEQ ID NO: 50 - DNA, 35 bp, synthetic construct
  misc_feature 1..35: note = Synthetic Construct
  source 1..35: mol_type = other DNA
gccattcacatcttatcaaagactgtaaattgttt

SEQ ID NO: 51 - DNA, 34 bp, synthetic construct
  misc_feature 1..34: note = Synthetic Construct
  source 1..34: mol_type = other DNA
atcctacaaaaaattgaacaacaatgccactttc

SEQ ID NO: 52 - DNA, 25 bp, synthetic construct
  misc_feature 1..25: note = Synthetic Construct
  source 1..25: mol_type = other DNA
acatggagctacagttcagatgtgc

SEQ ID NO: 53 - DNA, 27 bp, synthetic construct
  misc_feature 1..27: note = Synthetic Construct
  source 1..27: mol_type = other DNA
gcctgttgcacgagcccattactagat

SEQ ID NO: 54 - DNA, 34 bp, synthetic construct
  misc_feature 1..34: note = Synthetic Construct
  source 1..34: mol_type = other DNA
ggcaattacttgtttctttgtgcaattacttgtt

SEQ ID NO: 55 - DNA, 28 bp, synthetic construct
  misc_feature 1..28: note = Synthetic Construct
  source 1..28: mol_type = other DNA
gttttgaatgctcaagagaagtactagt

SEQ ID NO: 56 - DNA, 30 bp, synthetic construct; identical to SEQ ID NO: 48
  misc_feature 1..30: note = Synthetic Construct
  source 1..30: mol_type = other DNA

SEQ ID NO: 57 - DNA, 28 bp, synthetic construct
  misc_feature 1..28: note = Synthetic Construct
  source 1..28: mol_type = other DNA
ttatgtcttggtccaaagcccctttttg

SEQ ID NO: 58 - DNA, 30 bp, synthetic construct
  misc_feature 1..30: note = Synthetic Construct
  source 1..30: mol_type = other DNA
tccacgtcaggaacttagacatgcaactat